SEQUENCE LISTING (215 sequences)

SEQ ID NO: 1 - RNA, 15 nt, Artificial Sequence
  Synthetic Polynucleotide
cagucagaauaaugu

SEQ ID NO: 2 - protein, 684 aa, Homo sapiens
MSAIPAEESDQLLIRPLGAGQEVGRSCIILEFKGRKIMLDCGIHPGLEGMDALPYIDLIDPAEIDLLLISHFHLDHCGALPWFLQKTSFKGRTFMTHATKAIYRWLLSDYVKVSNISADDMLYTETDLEESMDKIETINFHEVKEVAGIKFWCYHAGHVLGAAMFMIEIAGVKLLYTGDFSRQEDRHLMAAEIPNIKPDILIIESTYGTHIHEKREEREARFCNTVHDIVNRGGRGLIPVFALGRAQELLLILDEYWQNHPELHDIPIYYASSLAKKCMAVYQTYVNAMNDKIRKQININNPFVFKHISNLKSMDHFDDIGPSVVMASPGMMQSGLSRELFESWCTDKRNGVIIAGYCVEGTLAKHIMSEPEEITTMSGQKLPLKMSVDYISFSAHTDYQQTSEFIRALKPPHVILVHGEQNEMARLKAALIREYEDNDEVHIEVHNPRNTEAVTLNFRGEKLAKVMGFLADKKPEQGQRVSGILVKRNFNYHILSPCDLSNYTDLAMSTVKQTQAIPYTGPFNLLCYQLQKLTGDVEELEIQEKPALKVFKNITVIQEPGMVVLEWLANPSNDMYADTVTTVILEVQSNPKIRKGAVQKVSKKLEMHVYSKRLEIMLQDIFGEDCVSVKDDSILSVTVDGKTANLNLETRTVECEEGSEDDESLREMVELAAQRLYEALTPVH

SEQ ID NO: 3 - protein, 285 aa, Homo sapiens
MARNAEKAMTALARFRQAQLEEGKVKERRPFLASECTELPKAEKWRRQIIGEISKKVAQIQNAGLGEFRIRDLNDEINKLLREKGHWEVRIKELGGPDYGKVGPKMLDHEGKEVPGNRGYKYFGAAKDLPGVRELFEKEPLPPPRKTRAELMKAIDFEYYGYLDEDDGVIVPLEQEYEKKLRAELVEKWKAEREARLARGEKEEEEEEEEEINIYAVTEEESDEEGSQEKGGDDSQQKFIAHVPVPSQQEIEEALVRRKKMELLQKYASETLQAQSEEARRLLGY

SEQ ID NO: 4 - protein, 1304 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNARTYMDVMREQHLTKEEREIRQQLAEKAKAGELKVVNGAAASQPPSKRKRRWDQTADQTPGATPKKLSSWDQAETPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGREIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYELDYIL

SEQ ID NO: 5 - DNA, 5740 bp, Artificial Sequence
  Synthetic polynucleotide
aggtgccgccgccgccgccccggcccgcgccttcgcgccacttcgcgccctgcggcgaggccgaggggtggggacggtcccccacgccgcccgtgcctctccacggagccccgagctcggaccgggccgggcgggccacgccgcacccccggcctggggcctcgggccggagtggcgcggaggctggcgaagatggtggcggctgctgcggtgagtgcgcccgccccgccgccggctcagggaaacctgttgtgcgctaagcggagcggcggcggcggcgggacgggcgggagggcgggggccgcgggccgggtgggtctcgggccgttgggcgccggcgtggggcggccgtcctggcgcgcacgcgggccggagggggcccgagggggcggcggcccgggccccggagaggcccgcccggcgcacacaatggccctcgggaggcttcgcacgaggccggcggccgtcccgggagcggcgggagccgggcggggcgcgcggccgccgtgcggggaaagtttctcccgggggcgagagttaaagcgcctccagaacaaagcggcggcggcacatggggcaggccgcgggccgggagggggcgcgcccacgaggaacctgcgcgcgggcggcgtgggcgggcggcactcggcgtccccgaaactttgtgcgcgccgggccgggctccgggacgcgggagctgcggggcgacggggccggggcgccaccccgcgctccgcgtgggctttgtgtgcggccgcgtgggcagctcccctcgcgggcgaggcgacccctgcggtccgcccgggcggcccgggacgccccggcccgccctcggggcggcgcggagccccggacgggcgctggctgcgagcgcggcgcgggagggccgggcgggttcggcaggagcggcggccccgccgccatgttcctgcggggcgggctgcgcgcgccgagggcgggggggggggggacatggcggcgactgcgcgccgccgccgattgttcccggcttaggcctcgggccgcgtgcggcgagcgccctgtccgcccccctccctcccccggagcctggccggggggcgcgcgggacacaaaggacggccggcgtgtccggagcctcgccacgctcgggcctccgcgccgccgctcgccgcgcagccgcccagaaacgggcgggggttgggggggggggggagtctccgcgtccggcgcagcgcggcccggctctgacctgccgccccctggcggccgcgcggggaacccacaagggccgcctgcccccttgtgcgacatgtgctgccggcccgggctccatgagcgtggcgggtacttggcaggatcccgtgttcccgtccggttttctcttgataagctggcgcggaggggagaaaactggagagcgcagcagggctcgtggttcttaggtgaccagagggaagaaaatgagcaacgtgccacgaggatcacagcagttggggaaacaactctttatcccggcgtgcagccacgaggtcctgattgggggaggggtggtgaagactagtcctggttgccctttttctctagcttgagctggacagtagagttcaggcttgctgctactcatcttgcagtatttcaggggcacattaattcgtgcacggatgtgaatcttggtgggttttcaactttattttgttacagcgttttccacccaatacattgttgccctcaggaccttgtggtcctggctctctctttttttttttttttttttttttttttggcaaatggatagaattgccccttaggaagattaagagaagagcaacttggaatggggctcggaaagtggtagtaattttgagcaaatgtgcttaccctttctctgtcttcaccccacccccaaccctagtcatacacgtggacctagcagcacccgaagcattgcccaaggatacttgctgagaaggaagtttgcctgagtgggtgagtatattctagttttgtagctaaaacttcttttgagacctttggttttcacttttgtctttgagcttgtacaacattcttggtcttttaaggtatgaaataaatacagtttgaactcttgtcataaatgagtgagccacattttaacgtagtaaatctacagtggcttttggacactaaccaaatagttgtagacccttcaagttggagtcatagagtatttctaatttggggtgatactaagacttttttatgttttatgactaataaacttgaaaatgactaaacaataatcattaatcttgtcgagtatctgacaatgtggaggacagaagaaagggattgctgcctggtcaatgtgaggctttcctctaaaggtagtaccaagtcagactgccttcctgataacaggtctcattttgtaggacccttattgtgtgttttttggggaagcctactgtaaaagccaacattttaatgggattgtatcttatatttctttaaggagttttttttttttttttaaggcttacatgtgtccaatttggaacttctggctattggctcctcccctcccccccttgggtataagctgtaattgatgtttgtgacagaatttagagctttggctttttcctttttgtctaattatttatttcaaatttagcaggaataaagtgaacctcaccttgggactgaagctgtgaccagtcagaataatgtcaaagtgcttacagtgcaggtagtgatgtgtgcatctactgcagtgagggcacttgtagcattatgctgacagctgcctcggtgggagccacagtgggcgctgcctcgggcggcactggctgcgtccagtcgtcggtcagtcggtcgcggggagggcctgctggtgctgcgtgctttttgttctaaggtgcatctagtgcagatagtgaagtagactagcatctactgccctaagtgctccttctggcataagaagttatgtcctcatccaatccaagtcaagcaagcatgtaggggtctctccatagttgtgtttgcagccctctgttagttttgcatagttgcactacaagaagaatgtagttgtgcaaatctatgcaaaactgatggtggcctgctatttacttcaagtgttgtttttttttaaactaattttgtatttttattgtgtcgatgtagagcctgcgtggtgtgtgtgatgtgacagcttctgtagcactaaagtgcttatagtgcaggtagtgtgtagccatctactgcattacgagcacttaaagtactgccagctgtagaactccagcctcgcctggccatcgcccagccaactgtcctgttattgagcactggtctatggttagttttgcaggtttgcatccagctgtataatattctgctgtgcaaatccatgcaaaactgactgtggtggtgaaaagtctgtagagaagtaagggaaaatcaaacccctttctacacaggttgggatttgtcgcaatgctgtgtttctctgtatggtattgcacttgtcccggcctgttgagtttggtggggattgtgaccagaagatgtgaaaatgacaatattgctgaagatgccgatttccactgtaaaaatgttcacgagctgatggagtgtatgaacacattctgtaaactgtgatttctattgctttcgttggtgtgtaaaacgggatttttttaattgaaatttgtgtttttaaattgtggtttgtttttactgaatcatttcattttcgtgtttgctcgctggatttagtaagaagttgtgtgtgggggcttaggtgaagttaaactgccttttgaacctggtatgacttcaggtttattttactctaaaacccattaacatgagttaagaaaaatgttactttctttggatctctgtttattaaagtgtgacaattcagatggagtgttttctgataagattattcctaatccgtgagttcttaacagttaacaggtggtggccactctgttaatgtgcacaccctgtaagaaagaactatccttggactgttcttaccatgtgaccccctcaagggaaagatggcaaactgatggtcagtagagtgacaggtacacatgacactcgagtgctgggtctggagaagctgcagttagtatttaggataacagtatatataatatatagttctacatttgggcagcacagttggtttcaggctatgaataaaaatcattggagtgaaacctaaagaaaagtaaaattaataaggaagagctgctaaaatcaggtttaagctgacactatctacagagctaaagttttccataatatgttgctttttttaaaagaaatgcaagaagctgactgatggatccctgagctgccaggtaaggattccacaggcctgggtttgattgtgagtgaccagaattgtacaattattactaaaacagaaacatagtcacttctgactccattcttaacatttttggattaaaacttgtttcagatgactaatgaaaacttcctttaaaacgtgataaagctctaatgtcagcacgatccacatgatccatgtgcctcctataaagggaggggtctgtcaccagtgttacggattgaatgctacgtttatcttccaacataggaagcctgccaggtactttcttcaatatactaaattagaagtttaaaattaataaggtcttaattaggacctttggatgctgagcttgtggtagtaattgtgttcagaatgttttgaacttaaattgagatttaattttagtaaggaatgtgtgtcttagaggcctagtagtgaagaggagttttcctaggattttcctctctcctcccttaattaagctgctttctgcactaagggcttcacgcagcaacgcacttgttcagttccgcacagggggctttgctgactgtctgctctaaaactctgttggcaaaacagctgtggtctctcttagactttgattttgtagtaactagggcatatagttgctataagctttcaggaatgggggtgggttactaatgtcttatgttatatgaaagtagttaaatttatcctatattaagaaggaagcattattgagactatatactgttgattttactaaaaactctaagatgtccattttaacaggcaaacctagcacagaaaaccagctggattttccctgtgcatggtttgaagagtcagtcctacataattgctgacacaatgcaacctgcaactgcccggagaaagaacagttcactaaaatttgttgtatttatcatccaattctgttctgtaactggtaacactagtttgtctggctttagagaataggtgaatctctaaaacagtagaaacagctcagttgggcaagggccgttctagtagcatgcctgctccttggagttttccagattattttgtatagcctattccagattcttgtctaagattgctttgctctgtgcactcagaattggtgtgcctttctatttgtgtaaatgatatataatacattgctagttgtctaggatctattcaggaagtgcgtgctgtgaattttaaagtatgggaagattgttaacaagggtttcaatgttttgtttttttttttctggtaagctaaaatagaacattgtgaggtacctggtttattgtggttacagacttggaataatgttcattgttttgtgctaataaattgtgttaaaatttggtgtaacatatgagcatttttaaataaagttattttgtagcactgat

SEQ ID NO: 6 - DNA, 762 bp, Artificial Sequence
  Synthetic polynucleotide
gaataatgtcaaagtgcttacagtgcaggtagtgatgtgtgcatctactgcagtgagggcacttgtagcattatgctgacagctgcctcggtgggagccacagtgggcgctgcctcgggcggcactggctgcgtccagtcgtcggtcagtcggtcgcggggagggcctgctggtgctgcgtgctttttgttctaaggtgcatctagtgcagatagtgaagtagactagcatctactgccctaagtgctccttctggcataagaagttatgtcctcatccaatccaagtcaagcaagcatgtaggggtctctccatagttgtgtttgcagccctctgttagttttgcatagttgcactacaagaagaatgtagttgtgcaaatctatgcaaaactgatggtggcctgctatttacttcaagtgttgtttttttttaaactaattttgtatttttattgtgtcgatgtagagcctgcgtggtgtgtgtgatgtgacagcttctgtagcactaaagtgcttatagtgcaggtagtgtgtagccatctactgcattacgagcacttaaagtactgccagctgtagaactccagcctcgcctggccatcgcccagccaactgtcctgttattgagcactggtctatggttagttttgcaggtttgcatccagctgtataatattctgctgtgcaaatccatgcaaaactgactgtggtggtgaaaagtctgtagagaagtaagggaaaatcaaacccctttctacac

SEQ ID NO: 7 - DNA, 6756 bp, Homo sapiens
gaagctctcctcgcggggcgggccggccggccgcacccccggcctggggcctccggtcgtagtaaagcgcaggcgggcggggaggcgggagcaggagcccgcggccggccagccgaagatggtggcggctactcctcctggtgagtctgcccgcccctccggcgacggagggaaacctgttgtgtgcggcccgggtctggcgggcggggcggagcggcccggggcggactggcccggggcagcgtggcggcggcggcgtggccggggcgggtctcggccgttggccgccccggcgtgtggcagccgcatctggctgccccctcgctcgcccgcgggccggcggaggggggcagggccggggcgggagggtgggagggggcggcgtgcgcgtggcggccgcgcccgggacccgcgcagaccctgcctgggccgacccgaaggcgggtgggcggacggcgaacacaatggcccctcggggagaggacgtgcgaggcccgtgccttctccggggcccggggcgcgcgcggggcgtggggtctctgggtaggaaagtttctcccgagggcgagagttaaagcgcctccagaacaaagcggcggcggcggcggcacatggggcaggccgcgggccgggagggggcgcgcccacgaggtacctgcgcgccagcgggcggcgtggcgtgggcgggagcccgcggttccccaaactttgtacgcgcgagggtgggcggaggggcgccgagatcggcgcggcctgggcgccacccccgctccgcgtgggctttgttagcccgcgtgggcagcctcggggcggggcccgcaacttccccgccgtggccctcggaggaggccgcagtcggcctcagccgcggcgtggagccgcctgcgcccggccgcttgctgggagtgtggcgcgggagggccagcccggctcggcgggagcggcgtccccgccgccatgttcctgcggggcgggctgcacgggggtgagggcgggggacatggcggcgactgcgcgccgccgccgattgttcccggcttaggcctcgggccgcgtgcgacgggcaccgcggccgcggagcccccgcccctctgggccgggctcgggggggctgggggacacaaaggaggggcggcgcgcccgcgtccccgccgcactcgggcctcggcgccgccggtcgccgcgcggctgccgccgggaaacgggttgggggggttgccgcgtccggcggggcctgactctgacccgccgccccctggcggctacgcggagaatcgcagggccgcgctcccccttgtgcgacatgtgctgccggcccgggctccatgagcgtggcgggcactttgcagtctcgggtgttcctgcccggtcttctgttcctaaactgcagcaaagggaaaaggaactgaaaaaggcaggctcgtcgttgcaatatcaccaaaagagaaaattaacggcatgccatcaggaccacagcagttggagaaacaactctttatcccggcttgcagccacgaggtcttgattgggggaggggtggtgaagaatagtctgtgggctgctttttttttttccttttactggagctgtacagtggagtcggtgattgctgctgatcataatcaagtattttaggagcttatttagacatgtatctgatagctaaggatttttcaactttattctcttacgtatttttcaactgtaaattattgggcttttaaatcctgctagtattgctcgactcttactctcacaaatggatggaattaattgctgttaggaggttggaaaatagcaaatatagatttggacggtggtagtaattttgagcaaataatgttttatcttttttttccttatttttccctattccagtcatacacgtggacctaactgcaccagtagcttttctgagaatacttgctgaaaaggaagttttctggaatgggtaagtgtattctgattttcttgaacttttcttaaaaacaaatttttcttgctattaaagttgaataaataggattggtttcttagagagtaaaagtaggtgtttctttctttagacaatgtaccttttctgaaaaactaactcattaagtacggatttgctaattttaaggtagtaaaattacagtgtaaatattcctgtacatttttggaaactggcttatgcagtttacgaaatataattttagaccctcttttaagttgggtgataaagtagatataacctgagatgatagatttaaacaggatatttacgttctgctacaattgactgataacacttgaagtgtagtctgaacagtaattttgttaatcatttcaacaagtatttgctaagtggaagccagaagaggaggaaaatgttttgccacgtggatgtgaagatttcctctaaaaggtacacatggactaaattgcctttaaatgttccaaaattagttctcatttatttgcagtctcattttgttttgttttttttctctatgtgtcaatccatttgggagaggccagccattggaagagccaccacttccagtgctagttggatggttggttatgattgccttctgtaaagaattcttaaggcataaatacgtgtctaaatggacctcatatctttgagataattaaactaattttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcaaagtgcttacagtgcaggtagtgatatgtgcatctactgcagtgaaggcacttgtagcattatggtgacagctgcctcgggaagccaagttgggctttaaagtgcagggcctgctgatgttgagtgctttttgttctaaggtgcatctagtgcagatagtgaagtagattagcatctactgccctaagtgctccttctggcataagaagttatgtattcatccaataattcaagccaagcaagtatataggtgttttaatagtttttgtttgcagtcctctgttagttttgcatagttgcactacaagaagaatgtagttgtgcaaatctatgcaaaactgatggtggcctgctatttccttcaaatgaatgatttttactaattttgtgtacttttattgtgtcgatgtagaatctgcctggtctatctgatgtgacagcttctgtagcactaaagtgcttatagtgcaggtagtgtttagttatctactgcattatgagcacttaaagtactgctagctgtagaactccagcttcggcctgtcgcccaatcaaactgtcctgttactgaacactgttctatggttagttttgcaggtttgcatccagctgtgtgatattctgctgtgcaaatccatgcaaaactgactgtggtagtgaaaagtctgtagaaaagtaagggaaactcaaacccctttctacacaggttgggatcggttgcaatgctgtgtttctgtatggtattgcacttgtcccggcctgttgagtttggtggggattgtgaccagaagattttgaaaattaaatattactgaagatttcgacttccactgttaaatgtacaagatacatgaaatattaaagaaaatgtgtaactttttgtgtaaatacatcttgtcttgttttcattcaaaaacatttcacttttggggttgcgtgtcagatttggcagtataaattctggctatattttttgttgttagatttatttggctgttaagtattgcgatatgactaaacatactgtatacctgatgatcatctgtaaagttagagtatatctttttgctttctttggagttagtgttattccaggatattttacttaatctaaaagttaatttatgttgctcatatattactcaagtatttaaatttagagagaatgccgctctgtttaaagcaatgtgtaaagatgagtttttttaagcatggaatttagggttggggtacaatttgtttctattaagcaagtaccagtttaccaatacatgagtaactgaagtgtaactgttaaatgcttgtatactagtttttctttctgattgtcagtgatttataagctataaatgaccaaggtcctcagactgcttttagcatctgcaacttaaaaaaatgggagttagaaaaagaacaaatgctaaatagagtaacagttaaatgtatgtgtacactcttcccaaatgccaagagtgcagcggtggggtgagattcagatattcatttatttctaagtctgtagttaacatttatgttccctactccctacgtaagccagactttggcaacagtgatagttgattccaggcttatttgacttaaagtcactgaagtggaaactaagaagtggcagttagtgttttacccagcatttctgccttctctcttttcttcatgtgtttttgtctctagcctatgtgtatttgtgtagaataatgtgggatacctgaataatagatttaaaaggaccaagtggtaaaattgggcccaagctgaagtacaggcaaacttgatgtttgaaagataagttttgagaaatgtcattgtattttggagtaaaagaggctatcttagtaataagaaataaacttccataacactaggttagaccacccaataaatctagaaatcagcttttaaaaatattgtctgaagtctaacaaaagttttcacctctaatgtgttctttaagaaatttaaggaacttagccttggattcctgaatagaaaggtaagaattctatcattctggagttgatgaaaacataaattttcaggatgtgaaatgaacagtgatttataaaatggaaatcaaattgtacattagcagagttcttaagctttttgaattgaaggagacctaataattgtgtctttttggttatttagtgacaaacgtggctttcaaactatgcttaaaaagttccggctggacacggtggctcacacctataatcctagcacttggggaggctgaggcagacggattacctgaggtcaggagttcgagaccaacctggccgacatggtgaaacgctgtctctactaaaaatataaaaaattagccgggtgcagtggcgtgcacctgtaatcccagctactctggaggctgaggcaggagaatcacctgaacctgggaggtggaggtttcagtgagctgagatcctgccactgcactccagcctgggcgcaagaccaagacttaaacgcaaaaaaaaaaaaaaaaaaaaaaaaaaagtttcataatacagcatggtctggtagtttgcaaaatggtgtgcttttggggagatacactagcaatttttttaaaaactggaacagtgtgataggaagcctgctggatgatttcttaaatattctaaaatgtaagtcaaatatgttttaataacaaagacttaaatggcttttctccctagagactgaaactagtattcattgtgttcagaacttaattgggcttgaactgagatttaaatctaataaacaagttaataaatgtgtatgttttgttgtgggtttggtagtgatctgtggttctatagggtttaataggaattgcttttgatttgtttctggctttagaatgtgaggcaaattttacattcttggttctattaagattttcttaggcatgctaacatgccaacaaaaagccatgtaagtattgtataaaaagattcacattgttaatttagccattttgaaattcagatgagtgagcaagttgataatggcctcatctctgacctgagaaaaaacaactttgacccttgttcttaaaatgctttaaccttgaagttgcttgagacttaagaggtcatgttgctttaggtttaataaatagccttaactatttggaggggaaaaaatgggtcaacttttttttttttttttggcgtttgcatgtacaactttctatttttagcctatatttggaaagaaagcacttaacattttaggaattctttttaaagctgcttgcaaagtgttggtgattttactgaaaacttttgagatcttcattttacaggcagacctgtctaactacaagccagacttgggttttctcctgtagtttgaagacacactgactcctgacaaaatgcagcctgcaacttcctggagaacaactcagtgtcacattaaagtttattatgtatttaatgatacactgtttaattgacagttttgcatagtttgtctaactttagagaattaagagcctctcaactgagcagtaaaggtaaggagagctcaatctgcacagagccagtttttagtgtttgatggaaataagatcatcatgcccacttgagacttcagattattctttagcttagtggttgtatgagttacatcttattaaagtcgaaattaatgtagttttctgccttgataacatttcatatgtggtattagttttaaagggtcattaggaaaatgcacatattccatgaattttaagacccatagaaaagttgaagaatgcttaattttcttatccagtaatgtaaacacagagacagaacattgagatgtgcctagttctgtatttacagtttggtctggctgtttgagttctagcgcatttaatgttaataaataaaatactgcattttaaagctgttaagaaattgtccagaacgagaatattgaaataaaaacttcaaggt

SEQ ID NO: 8 - RNA, 59 nt, Artificial Sequence
  Synthetic polynucleotide
caaagugcuuacagugcagguagugauaugugcaucuacugcagugaaggcacuuguag

SEQ ID NO: 9 - RNA, 64 nt, Artificial Sequence
  Synthetic polynucleotide
uaaggugcaucuagugcagauagugaaguagauuagcaucuacugcccuaagugcuccuucugg

SEQ ID NO: 10 - RNA, 58 nt, Artificial Sequence
  Synthetic polynucleotide
aguuuugcauaguugcacuacaagaagaauguaguugugcaaaucuaugcaaaacuga

SEQ ID NO: 11 - RNA, 61 nt, Artificial Sequence
  Synthetic polynucleotide
aguuuugcagguuugcauccagcugugugauauucugcugugcaaauccaugcaaaacuga

SEQ ID NO: 12 - RNA, 58 nt, Artificial Sequence
  Synthetic polynucleotide
uaaagugcuuauagugcagguaguguuuaguuaucuacugcauuaugagcacuuaaag

SEQ ID NO: 13 - RNA, 59 nt, Artificial Sequence
  Synthetic polynucleotide
agguugggaucgguugcaaugcuguguuucuguaugguauugcacuugucccggccugu

SEQ ID NO: 14 - DNA, 25 bp, Artificial Sequence
  Synthetic polynucleotide
ggtcgacatggcccgaaatgcagaa

SEQ ID NO: 15 - DNA, 28 bp, Artificial Sequence
  Synthetic polynucleotide
cggatccctagtaccccaggagtcgctt

SEQ ID NO: 16 - DNA, 30 bp, Artificial Sequence
  Synthetic polynucleotide
ggtcgacatgtctgcgattcctgctgagga

SEQ ID NO: 17 - DNA, 29 bp, Artificial Sequence
  Synthetic polynucleotide
cggatcctcagtgaactggcgtcagggcc

SEQ ID NO: 18 - DNA, 30 bp, Artificial Sequence
  Synthetic polynucleotide
gcggccgctcagtgaactggcgtcagggcc

SEQ ID NO: 19 - DNA, 30 bp, Artificial Sequence
  Synthetic polynucleotide
cggaattccctagtcatacacgtggaccta

SEQ ID NO: 20 - DNA, 30 bp, Artificial Sequence
  Synthetic polynucleotide
cggaattcgtcagaataatgtcaaagtgct

SEQ ID NO: 21 - DNA, 29 bp, Artificial Sequence
  Synthetic polynucleotide
cgctcgagtcagtgctacaaaataacttt

SEQ ID NO: 22 - DNA, 31 bp, Artificial Sequence
  Synthetic polynucleotide
cgctcgagccaaactcaacaggccgggacaa

SEQ ID NO: 23 - DNA, 30 bp, Artificial Sequence
  Synthetic polynucleotide
cgctcgagcctagtcatacacgtggaccta

SEQ ID NO: 24 - DNA, 29 bp, Artificial Sequence
  Synthetic polynucleotide
tgcggccgccagtagatgcacacatcact

SEQ ID NO: 25 - DNA, 27 bp, Artificial Sequence
  Synthetic polynucleotide
gtgaccagtcccaataatgtcaaagtg

SEQ ID NO: 26 - DNA, 18 bp, Artificial Sequence
  Synthetic polynucleotide
agcttcagtcccaaggtg

SEQ ID NO: 27 - DNA, 25 bp, Artificial Sequence
  Synthetic polynucleotide
gtgaccagtcacaataatgtcaaag

SEQ ID NO: 28 - DNA, 19 bp, Artificial Sequence
  Synthetic polynucleotide
cagcttcagtcccaaggtg

SEQ ID NO: 29 - DNA, 25 bp, Artificial Sequence
  Synthetic polynucleotide
gtgaccagtccgaataatgtcaaag

SEQ ID NO: 30 - DNA, 18 bp, Artificial Sequence
  Synthetic polynucleotide
agcttcagtcccaaggtg

SEQ ID NO: 31 - DNA, 26 bp, Artificial Sequence
  Synthetic polynucleotide
tgtgaccagtaagaataatgtcaaag

SEQ ID NO: 32 - DNA, 19 bp, Artificial Sequence
  Synthetic polynucleotide
gcttcagtcccaaggtgag

SEQ ID NO: 33 - DNA, 29 bp, Artificial Sequence
  Synthetic polynucleotide
tgtgaccagtgggaataatgtcaaagtgc

SEQ ID NO: 34 - DNA, 19 bp, Artificial Sequence
  Synthetic polynucleotide
gcttcagtcccaaggtgag

SEQ ID NO: 35 - DNA, 32 bp, Artificial Sequence
  Synthetic polynucleotide
aattatttatttcaaatttagcaggaataaag

SEQ ID NO: 36 - DNA, 21 bp, Artificial Sequence
  Synthetic polynucleotide
tctgtcacaaacatcaattac

SEQ ID NO: 37 - DNA, 18 bp, Artificial Sequence
  Synthetic polynucleotide
caggttgggatttgtcgc

SEQ ID NO: 38 - DNA, 19 bp, Artificial Sequence
  Synthetic polynucleotide
cccttacttctctacagac

SEQ ID NO: 39 - DNA, 38 bp, Artificial Sequence
  Synthetic polynucleotide
aagagaaattatttatttcaaatttagcaggaataaag

SEQ ID NO: 40 - DNA, 28 bp, Artificial Sequence
  Synthetic polynucleotide
cattccaaaaagccaaagctctaaattc

SEQ ID NO: 41 - DNA, 27 bp, Artificial Sequence
  Synthetic polynucleotide
cattccgaaaatcaaacccctttctac

SEQ ID NO: 42 - DNA, 25 bp, Artificial Sequence
  Synthetic polynucleotide
aagagaacagacttttcaccaccac

SEQ ID NO: 43 - DNA, 29 bp, Artificial Sequence
  Synthetic polynucleotide
tttccatttgaaggcctgtggagccctgc

SEQ ID NO: 44 - DNA, 18 bp, Artificial Sequence
  Synthetic polynucleotide
tgactgatcaacagaagg

SEQ ID NO: 45 - DNA, 30 bp, Artificial Sequence
  Synthetic polynucleotide
cggaattctaaagtgaacctcaccttggga

SEQ ID NO: 46 - DNA, 32 bp, Artificial Sequence
  Synthetic polynucleotide
cggaattctttgtctaattatttatttcaaat

SEQ ID NO: 47 - DNA, 30 bp, Artificial Sequence
  Synthetic polynucleotide
cggaattctttgtgacagaatttagagctt

SEQ ID NO: 48 - DNA, 30 bp, Artificial Sequence
  Synthetic polynucleotide
cggaattccttctggctattggctcctccc

SEQ ID NO: 49 - DNA, 29 bp, Artificial Sequence
  Synthetic polynucleotide
cggaattcaaggcttacatgtgtccaatt

SEQ ID NO: 50 - DNA, 29 bp, Artificial Sequence
  Synthetic polynucleotide
cggaattctttgtaggacccttattgtgt

SEQ ID NO: 51 - DNA, 28 bp, Artificial Sequence
  Synthetic polynucleotide
cggaattctatctgacaatgtggaggac

SEQ ID NO: 52 - DNA, 29 bp, Artificial Sequence
  Synthetic polynucleotide
cggaattctatttctaatttggggtgata

SEQ ID NO: 53 - DNA, 31 bp, Artificial Sequence
  Synthetic polynucleotide
cggaattcaataaatacagtttgaactcttg

SEQ ID NO: 54 - DNA, 34 bp, Artificial Sequence
  Synthetic polynucleotide
cggaattcgtgagtatattctagttttgtagcta

SEQ ID NO: 55 - DNA, 25 bp, Artificial Sequence
  Synthetic polynucleotide
caccgaataaagtgaacctcacctt

SEQ ID NO: 56 - DNA, 25 bp, Artificial Sequence
  Synthetic polynucleotide
aaacaaggtgaggttcactttattc

SEQ ID NO: 57 - DNA, 189 bp, Artificial Sequence
  Synthetic polynucleotide
acagaatttagagctttggctttttcctttttgtctaattatttatttcaaatttagcaggaataaagtgaacctcaccttgggactgaagctgtgaccagtcccaataatgtcaaagtgcttacagtgcaggtagtgatgtgtgcatctactgcagtgagggcacttgtagcattatgctgacagctg

SEQ ID NO: 58 - DNA, 23 bp, Artificial Sequence
  Synthetic polynucleotide
ctacctgcactataagcacttta

SEQ ID NO: 59 - DNA, 23 bp, Artificial Sequence
  Synthetic polynucleotide
ctatctgcactagatgcacctta

SEQ ID NO: 60 - DNA, 23 bp, Artificial Sequence
  Synthetic polynucleotide
tcagttttgcatagatttgcaca

SEQ ID NO: 61 - DNA, 21 bp, Artificial Sequence
  Synthetic polynucleotide
caggccgggacaagtgcaata

SEQ ID NO: 62 - DNA, 19 bp, Artificial Sequence
  Synthetic polynucleotide
gctcgtggttcttaggtga

SEQ ID NO: 63 - DNA, 19 bp, Artificial Sequence
  Synthetic polynucleotide
gcagcaagcctgaactcta

SEQ ID NO: 64 - DNA, 23 bp, Artificial Sequence
  Synthetic polynucleotide
ggtgagtatattctagttttgta

SEQ ID NO: 65 - DNA, 24 bp, Artificial Sequence
  Synthetic polynucleotide
tcgacaagattaatgattattgtt

SEQ ID NO: 66 - DNA, 23 bp, Artificial Sequence
  Synthetic polynucleotide
ctcatccaatccaagtcaagcaa

SEQ ID NO: 67 - DNA, 23 bp, Artificial Sequence
  Synthetic polynucleotide
acaccacgcaggctctacatcga

SEQ ID NO: 68 - DNA, 22 bp, Artificial Sequence
  Synthetic polynucleotide
tggtttgaagagtcagtcctac

SEQ ID NO: 69 - DNA, 19 bp, Artificial Sequence
  Synthetic polynucleotide
agcacgcacttcctgaata

SEQ ID NO: 70 - DNA, 27 bp, Artificial Sequence
  Synthetic polynucleotide
agtgctacagaagctgtcacatcacac

SEQ ID NO: 71 - DNA, 27 bp, Artificial Sequence
  Synthetic polynucleotide
ctggagttctacagctggcagtacttt

SEQ ID NO: 72 - DNA, 23 bp, Artificial Sequence
  Synthetic polynucleotide
acaccacgcaggctctacatcga

SEQ ID NO: 73 - DNA, 18 bp, Artificial Sequence
  Synthetic polynucleotide
tggcaggcttcctatgtt

SEQ ID NO: 74 - DNA, 19 bp, Artificial Sequence
  Synthetic polynucleotide
gcaggcttcctatgttgga

SEQ ID NO: 75 - DNA, 22 bp, Artificial Sequence
  Synthetic polynucleotide
cttcttactaaatccagcgagc

SEQ ID NO: 76 - DNA, 24 bp, Artificial Sequence
  Synthetic polynucleotide
gtgtagaaaggggtttgattttcc

SEQ ID NO: 77 - DNA, 24 bp, Artificial Sequence
  Synthetic polynucleotide
aaccatagaccagtgctcaataac

SEQ ID NO: 78 - RNA, 23 nt, Artificial Sequence
  Synthetic polynucleotide
uuuggcuuuuuccuuuuugucua

SEQ ID NO: 79 - RNA, 23 nt, Artificial Sequence
  Synthetic polynucleotide
uagagaaguaagggaaaaucaaa

SEQ ID NO: 80 - RNA, 19 nt, Artificial Sequence
  Synthetic polynucleotide
ucaugaaggccaucgauuu

SEQ ID NO: 81 - RNA, 21 nt, Artificial Sequence
  Synthetic polynucleotide
guaaagaagucccaggaaauu

SEQ ID NO: 82 - RNA, 21 nt, Artificial Sequence
  Synthetic polynucleotide
ucggaaagucagagacguauu

SEQ ID NO: 83 - RNA, 19 nt, Artificial Sequence
  Synthetic polynucleotide
agcuaaagaugccgaguua

SEQ ID NO: 84 - RNA, 19 nt, Artificial Sequence
  Synthetic polynucleotide
gaauuaaaugggacgagua

SEQ ID NO: 85 - RNA, 19 nt, Artificial Sequence
  Synthetic polynucleotide
guaccagaccuacgugaau

SEQ ID NO: 86 - RNA, 19 nt, Artificial Sequence
  Synthetic polynucleotide
ugagaagcgugaagagcga

SEQ ID NO: 87 - RNA, 19 nt, Artificial Sequence
  Synthetic polynucleotide
ccaaagcgccugacgauuu

SEQ ID NO: 88 - RNA, 19 nt, Artificial Sequence
  Synthetic polynucleotide
gauuguggauugcuaauua

SEQ ID NO: 89 - RNA, 19 nt, Artificial Sequence
  Synthetic polynucleotide
gugauaacuaugcgacguu

SEQ ID NO: 90 - RNA, 19 nt, Artificial Sequence
  Synthetic polynucleotide
aggcucagacugcggaauu

SEQ ID NO: 91 - RNA, 19 nt, Artificial Sequence
  Synthetic polynucleotide
cugcuuguccagagggcga

SEQ ID NO: 92 - RNA, 19 nt, Artificial Sequence
  Synthetic polynucleotide
gguuaaggauagugccaca

SEQ ID NO: 93 - RNA, 19 nt, Artificial Sequence
  Synthetic polynucleotide
gacuguaacuggagacgua

SEQ ID NO: 94 - RNA, 19 nt, Artificial Sequence
  Synthetic polynucleotide
acaaauaucccaacgcuga

SEQ ID NO: 95 - RNA, 19 nt, Artificial Sequence
  Synthetic polynucleotide
gcgaaaccucaaugggcga

SEQ ID NO: 96 - RNA, 19 nt, Artificial Sequence
  Synthetic polynucleotide
gaccuaacgucauguugaa

SEQ ID NO: 97 - RNA, 19 nt, Artificial Sequence
  Synthetic polynucleotide
caaacaagauuacgguaca

SEQ ID NO: 98 - RNA, 19 nt, Artificial Sequence
  Synthetic polynucleotide
acagugacgaagagcgaua

SEQ ID NO: 99 - DNA, 22 bp, Artificial Sequence
  Synthetic polynucleotide
gttacctggatgaagatgatgg

SEQ ID NO: 100 - DNA, 18 bp, Artificial Sequence
  Synthetic polynucleotide
tggttgccttcctcatca

SEQ ID NO: 101 - DNA, 20 bp, Artificial Sequence
  Synthetic polynucleotide
caacttccagtgctcgtaaa

SEQ ID NO: 102 - DNA, 20 bp, Artificial Sequence
  Synthetic polynucleotide
cattatgtgacctggagtcg

SEQ ID NO: 103 - DNA, 20 bp, Artificial Sequence
  Synthetic polynucleotide
tgaagggaaagaacttgagg

SEQ ID NO: 104 - DNA, 19 bp, Artificial Sequence
  Synthetic polynucleotide
ctttgtcttgtgagccgat

SEQ ID NO: 105 - DNA, 22 bp, Artificial Sequence
  Synthetic polynucleotide
caagacaagaagacagacacct

SEQ ID NO: 106 - DNA, 21 bp, Artificial Sequence
  Synthetic polynucleotide
gagagcaaagacaggaatgag

SEQ ID NO: 107 - DNA, 20 bp, Artificial Sequence
  Synthetic polynucleotide
tcggaagaaacccaagatgt

SEQ ID NO: 108 - DNA, 20 bp, Artificial Sequence
  Synthetic polynucleotide
gccacagggaaggaacttag

SEQ ID NO: 109 - DNA, 24 bp, Artificial Sequence
  Synthetic polynucleotide
cccgacctatgtagagaatcagat

SEQ ID NO: 110 - DNA, 20 bp, Artificial Sequence
  Synthetic polynucleotide
ttggagtgggtggagaggat

SEQ ID NO: 111 - DNA, 20 bp, Artificial Sequence
  Synthetic polynucleotide
tgtttatgtgcctggagttg

SEQ ID NO: 112 - DNA, 20 bp, Artificial Sequence
  Synthetic polynucleotide
ggtgtctgattgatggtgct

SEQ ID NO: 113 - DNA, 20 bp, Artificial Sequence
  Synthetic polynucleotide
ttcccaaacagcctcttatc

SEQ ID NO: 114 - DNA, 22 bp, Artificial Sequence
  Synthetic polynucleotide
ggaagactaaggagaagaacca

SEQ ID NO: 115 - DNA, 20 bp, Artificial Sequence
  Synthetic polynucleotide
ttctatgccaggtggagttc

SEQ ID NO: 116 - DNA, 20 bp, Artificial Sequence
  Synthetic polynucleotide
gtcattgggagcatcaccta

SEQ ID NO: 117 - DNA, 22 bp, Artificial Sequence
  Synthetic polynucleotide
tcctgagcaaagacagagtatc

SEQ ID NO: 118 - DNA, 20 bp, Artificial Sequence
  Synthetic polynucleotide
gagcaatggcgatgtaagac

SEQ ID NO: 119 - DNA, 20 bp, Artificial Sequence
  Synthetic polynucleotide
tgtgaaggacattccacctc

SEQ ID NO: 120 - DNA, 20 bp, Artificial Sequence
  Synthetic polynucleotide
gtcactgagagcaaagtccc

SEQ ID NO: 121 - DNA, 20 bp, Artificial Sequence
  Synthetic polynucleotide
tagttccagaggtccgatga

SEQ ID NO: 122 - DNA, 20 bp, Artificial Sequence
  Synthetic polynucleotide
cttgtggagttacctggctt

SEQ ID NO: 123 - DNA, 21 bp, Artificial Sequence
  Synthetic polynucleotide
gcacctcttctcagtctcaga

SEQ ID NO: 124 - DNA, 20 bp, Artificial Sequence
  Synthetic polynucleotide
gaggaggaaggtgagtagga

SEQ ID NO: 125 - DNA, 21 bp, Artificial Sequence
  Synthetic polynucleotide
ggaaacaagacacaagtcctc

SEQ ID NO: 126 - DNA, 21 bp, Artificial Sequence
  Synthetic polynucleotide
gcatctatttctgggactgac

SEQ ID NO: 127 - DNA, 20 bp, Artificial Sequence
  Synthetic polynucleotide
tggaccagaaggatgtcaac

SEQ ID NO: 128 - DNA, 20 bp, Artificial Sequence
  Synthetic polynucleotide
gcaatgtcatacacacccat

SEQ ID NO: 129 - DNA, 20 bp, Artificial Sequence
  Synthetic polynucleotide
cggaaggactcagaacttgt

SEQ ID NO: 130 - DNA, 20 bp, Artificial Sequence
  Synthetic polynucleotide
tggcagaacctgagatgaga

SEQ ID NO: 131 - DNA, 21 bp, Artificial Sequence
  Synthetic polynucleotide
gactgtgataactatgcgacg

SEQ ID NO: 132 - DNA, 20 bp, Artificial Sequence
  Synthetic polynucleotide
ctggctgggtaacaggagtt

SEQ ID NO: 133 - DNA, 20 bp, Artificial Sequence
  Synthetic polynucleotide
tcccaaagagagatgtgtga

SEQ ID NO: 134 - DNA, 20 bp, Artificial Sequence
  Synthetic polynucleotide
actggatgctgagaggatga

SEQ ID NO: 135 - DNA, 23 bp, Artificial Sequence
  Synthetic polynucleotide
ggtgagtatattctagttttgta

SEQ ID NO: 136 - DNA, 24 bp, Artificial Sequence
  Synthetic polynucleotide
tcgacaagattaatgattattgtt

SEQ ID NO: 137 - DNA, 21 bp, Artificial Sequence
  Synthetic polynucleotide
aaggcttacatgtgtccaatt

SEQ ID NO: 138 - DNA, 20 bp, Artificial Sequence
  Synthetic polynucleotide
cacttagggcagtagatgct

SEQ ID NO: 139 - DNA, 22 bp, Artificial Sequence
  Synthetic polynucleotide
tggtttgaagagtcagtcctac

SEQ ID NO: 140 - DNA, 19 bp, Artificial Sequence
  Synthetic polynucleotide
agcacgcacttcctgaata

SEQ ID NO: 141 - RNA, 86 nt, Artificial Sequence
  Synthetic polynucleotide
uuagcaggaauaaagugaaccucaccuugggacugaagcugugaccagucagaauaaugucaaagugcuuacagugcagguaguga

SEQ ID NO: 142 - RNA, 76 nt, Artificial Sequence
  Synthetic polynucleotide
acagguugggauuugucgcaaugcuguguuucucuguaugguauugcacuugucccggccuguugaguuugguggg

SEQ ID NO: 143 - RNA, 77 nt, Artificial Sequence
  Synthetic polynucleotide
cagucagaauaaugucaaagugcuuacagugcagguagugaugugugcaucuacugcagugagggcacuuguagcau

SEQ ID NO: 144 - RNA, 34 nt, Artificial Sequence
  Synthetic polynucleotide
cagucagaauaaugucaaagugcuuacagugcag

SEQ ID NO: 145 - RNA, 54 nt, Artificial Sequence
  Synthetic polynucleotide
gaauuuagagcuuuggcuuuuuccuuuuugucuaauuauuuauuucaaauuuag

SEQ ID NO: 146 - RNA, 17 nt, Artificial Sequence
  Synthetic polynucleotide
uggucuaugguuaguuu

SEQ ID NO: 147 - RNA, 73 nt, Artificial Sequence
  Synthetic polynucleotide
aaacugacugugguggugaaaagucuguagagaaguaagggaaaaucaaaccccuuucuuucuacacagguug

SEQ ID NO: 148 - RNA, 14 nt, Artificial Sequence
  Synthetic polynucleotide
cggccuguugaguu

SEQ ID NO: 149 - RNA, 42 nt, Artificial Sequence
  Synthetic polynucleotide
agagcuuuggcuuuuuccuuuuugucuaauuauuuauuucaa

SEQ ID NO: 150 - RNA, 42 nt, Artificial Sequence
  Synthetic polynucleotide
agagcuuuggcuuuuuggaaugaagagaauuauuuauuucaa

SEQ ID NO: 151 - RNA, 42 nt, Artificial Sequence
  Synthetic polynucleotide
uuuccccaaacuaaaagggaaugaagagaugucugaaaagug

SEQ ID NO: 152 - RNA, 42 nt, Artificial Sequence
  Synthetic polynucleotide
uuuccccaaacuaaaagccuuacuucucuugucugaaaagug

SEQ ID NO: 153 - RNA, 42 nt, Artificial Sequence
  Synthetic polynucleotide
cagucagaauaaugucaaagugcuuacagugcagguagugau

SEQ ID NO: 154 - RNA, 41 nt, Artificial Sequence
  Synthetic polynucleotide
cagucagaauaaugucaaagugcuuacagugcagguaguga

SEQ ID NO: 155 - DNA, 33 bp, Homo sapiens
tttttcaactttattctcttacgtatttttcaa

SEQ ID NO: 156 - DNA, 32 bp, Gorilla gorilla
tttttcaactttattctcttacgtatttttca

SEQ ID NO: 157 - DNA, 33 bp, Pan troglodytes
tttttcaactttattctcttacgcatttttcaa

SEQ ID NO: 158 - DNA, 32 bp, Pongo borneo
tttttcaactttattctcttacgcatttttca

SEQ ID NO: 159 - DNA, 32 bp, Macaca mulatta; identical to SEQ ID NO: 158

SEQ ID NO: 160 - DNA, 32 bp, Simia hamadryas; identical to SEQ ID NO: 158

SEQ ID NO: 161 - DNA, 32 bp, Simia sciureus
tttttcaactttattctcttccgcatttttaa

SEQ ID NO: 162 - DNA, 33 bp, Callithrix jacchus
tttttcaactttattctcttccgcaatttttaa

SEQ ID NO: 163 - DNA, 32 bp, Carlito syrichta
ttttcaactttattctcttccacattttcgcc

SEQ ID NO: 164 - DNA, 32 bp, Balaenoptera acutorostrata
tttttcaactttattcttttacaccttttcac

SEQ ID NO: 165 - DNA, 32 bp, Tursiops truncatus
tttttcaactttatccttttacaccttttcac

SEQ ID NO: 166 - DNA, 33 bp, Atelerix albiventris
tttttcaactttgataccctcacgccttttcac

SEQ ID NO: 167 - DNA, 32 bp, Equus ferus
tttttaaactttattctcttagacgttttcac

SEQ ID NO: 168 - DNA, 32 bp, Ceratotherium simum
tttttaaactttattctcttagacattttcac

SEQ ID NO: 169 - DNA, 32 bp, Felis catus
tttttcaactttattctcttatacgtttacac

SEQ ID NO: 170 - DNA, 32 bp, Pteropus alecto
tttttctactttattctcttacacgttttcac

SEQ ID NO: 171 - DNA, 33 bp, Mustela putorius furo
gttttcaactttattctcttaacacgttttcac

SEQ ID NO: 172 - DNA, 33 bp, Canis lupus familiaris
tttttcaactttattctcttagcacgctttcac

SEQ ID NO: 173 - DNA, 32 bp, Nycticeius humeralis
tttttcaactttattttcttactaattttcac

SEQ ID NO: 174 - DNA, 32 bp, Sus scrofa
tttttcaactttattcttttacactttttcac

SEQ ID NO: 175 - DNA, 32 bp, Loxodonta africana
tttttcaagtttattcctctctgcatatttac

SEQ ID NO: 176 - DNA, 32 bp, Trichechus inunguis
tttttcgagtttattcttttccacatattcac

SEQ ID NO: 177 - DNA, 31 bp, Tenrec ecaudatus
tttttcagatttcttctcttcctcagtcgcc

SEQ ID NO: 178 - DNA, 33 bp, Mus musculus
ctttggctttttcctttttgtctaattatttat

SEQ ID NO: 179 - DNA, 33 bp, Cavia porcellus
tggtggttattttttgtctaaagaactttctat

SEQ ID NO: 180 - DNA, 33 bp, Ochotona daurica
ttgttggttttttttttttttttgattgttgct

SEQ ID NO: 181 - DNA, 33 bp, Sorex araneus
cttttgatttttctcatttgcacgcagtttgat

SEQ ID NO: 182 - DNA, 30 bp, Artificial Sequence
  Synthetic polynucleotide
tttttcaactttattctcttaccttttcac

SEQ ID NO: 183 - DNA, 34 bp, Homo sapiens
tgaaaagtctgtagaaaagtaagggaaactcaaa

SEQ ID NO: 184 - DNA, 34 bp, Gorilla gorilla; identical to SEQ ID NO: 183

SEQ ID NO: 185 - DNA, 34 bp, Pan troglodytes; identical to SEQ ID NO: 183

SEQ ID NO: 186 - DNA, 34 bp, Pongo borneo
tgaaaagtctgtagaaaagtaagggaaaatcaaa

SEQ ID NO: 187 - DNA, 34 bp, Macaca mulatta; identical to SEQ ID NO: 186

SEQ ID NO: 188 - DNA, 34 bp, Simia hamadryas; identical to SEQ ID NO: 186

SEQ ID NO: 189 - DNA, 34 bp, Simia sciureus; identical to SEQ ID NO: 186

SEQ ID NO: 190 - DNA, 34 bp, Callithrix jacchus; identical to SEQ ID NO: 186

SEQ ID NO: 191 - DNA, 34 bp, Carlito syrichta; identical to SEQ ID NO: 186

SEQ ID NO: 192 - DNA, 34 bp, Balaenoptera acutorostrata; identical to SEQ ID NO: 183

SEQ ID NO: 193 - DNA, 34 bp, Tursiops truncatus; identical to SEQ ID NO: 183

SEQ ID NO: 194 - DNA, 35 bp, Cavia porcellus
tgaaaagtctgtagaaaaagtgagggaaaatcaaa

SEQ ID NO: 195 - DNA, 34 bp, Tenrec ecaudatus
tgaaaattctgtagaaaattcagggaaaatcaaa

SEQ ID NO: 196 - DNA, 34 bp, Atelerix albiventris
tgaaaagtctgtagaaatgtaagggaaaatcaaa

SEQ ID NO: 197 - DNA, 34 bp, Equus ferus; identical to SEQ ID NO: 186

SEQ ID NO: 198 - DNA, 34 bp, Felis catus; identical to SEQ ID NO: 186

SEQ ID NO: 199 - DNA, 34 bp, Pteropus alecto; identical to SEQ ID NO: 186

SEQ ID NO: 200 - DNA, 34 bp, Trichechus inunguis; identical to SEQ ID NO: 186

SEQ ID NO: 201 - DNA, 34 bp, Ochotona daurica
tgaaaagtctgtagacgaggaagggagcatccaa

SEQ ID NO: 202 - DNA, 34 bp, Ceratotherium simum
tgaaaagtctgtagaaaaggaagggaaaatcaaa

SEQ ID NO: 203 - DNA, 34 bp, Mustela putorius furo; identical to SEQ ID NO: 186

SEQ ID NO: 204 - DNA, 34 bp, Canis lupus familiaris; identical to SEQ ID NO: 183

SEQ ID NO: 205 - DNA, 34 bp, Nycticeius humeralis
tgaaaagtctgtagcaaagtaagggaaaatcaaa

SEQ ID NO: 206 - DNA, 34 bp, Sus scrofa; identical to SEQ ID NO: 186

SEQ ID NO: 207 - DNA, 34 bp, Loxodonta africana; identical to SEQ ID NO: 186

SEQ ID NO: 208 - DNA, 34 bp, Sorex araneus; identical to SEQ ID NO: 186

SEQ ID NO: 209 - DNA, 34 bp, Mus musculus
tgaaaagtctgtagagaagtaagggaaaatcaaa

SEQ ID NO: 210 - DNA, 34 bp, Artificial Sequence; identical to SEQ ID NO: 186
  Synthetic polynucleotide

SEQ ID NO: 211 - RNA, 59 nt, Artificial Sequence
  Synthetic polynucleotide
guaucugauagcuaaggauuuuucaacuuuauucucuuacguauuuuucaacuguaaau

SEQ ID NO: 212 - RNA, 20 nt, Artificial Sequence
  Synthetic polynucleotide
cacuguucuaugguuaguuu

SEQ ID NO: 213 - RNA, 76 nt, Artificial Sequence
  Synthetic polynucleotide
aaacugacugugguagugaaaagucuguagaaaaguaagggaaacucaaaccccuuucuacacagguugggaucgg

SEQ ID NO: 214 - RNA, 19 nt, Artificial Sequence
  Synthetic polynucleotide
cuugucccggccuguugag

SEQ ID NO: 215 - DNA, 5740 bp, Artificial Sequence; identical to SEQ ID NO: 5
  Synthetic polynucleotide

(19) United States
(12) Patent Application Publication
(10) Pub. No.: US 2018/0156780 A1
(43) Pub. Date: Jun. 7, 2018

(54) COMPOSITIONS AND METHODS FOR MODULATING ONCOGENIC MIRNA

(71) Applicant: Children's Medical Center Corporation, Boston, MA (US)

(72) Inventors: Richard I. Gregory, Brookline, MA (US); Peng Du, Boston, MA (US)

(21) Appl. No.: 15/576,448

(22) PCT Filed: May 26, 2016
(86) PCT No.: PCT/US16/34441

§ 371 Date: Nov. 22, 2017

Related U.S. Application Data
(60) Provisional application No. 62/166,180, filed on May 26, 2015.

(51) Int. Cl. G01N 33/50; A61P 35/00; C12N 15/113

(52) U.S. Cl. CPC G01N 33/5023; C12N 2310/141; C12N 15/113; A61P 35/00

(57) ABSTRACT

Provided herein are compositions and methods related to modulation of progenitor microRNAs (pro-miRNAs), such as for the treatment of cancer.

### RELATED APPLICATION

This application claims the benefit under 35 U.S.C. § 119(e) of U.S. provisional application filed May 26, 2015, entitled “COMPOSITIONS AND METHODS FOR MODULATING ONCOGENIC MIRNA”, Ser. No. 62/166,180, the contents of which are incorporated by reference herein in their entirety.

### GOVERNMENT SUPPORT

This invention was made with government support under R01GM086386 awarded by the National Institutes of Health. The government has certain rights in the invention.

### BACKGROUND OF INVENTION

The precise control of miR-17˜92 microRNA (miRNA) is essential for normal development and overexpression of certain miRNAs from this cluster is oncogenic. There remains a need for means of modulating miR-17˜92 microRNA biogenesis.

### SUMMARY OF THE INVENTION

The disclosure is based, in part, on a study that shows there is a third step in biogenesis of the miR-17˜92 microRNA. A novel miRNA biogenesis intermediate, termed ‘progenitor-miRNA’ (pro-miRNA), was identified that is an efficient substrate for Microprocessor (which comprises the ribonuclease DROSHA and its co-factor, the double-stranded RNA-binding protein DGCR8). An autoinhibitory 5′ RNA fragment was found to be cleaved to generate pro-miRNA and selectively license Microprocessor-mediated production of pre-miR-17, -18a, -19a, 20a, and -19b. Using genetic, biochemical, and structural methods, two complementary cis-regulatory repression domains were found to be required for the formation of this inhibitory RNA conformation. It was determined that the endonuclease CPSF3 (CPSF73), and the Spliceosome-associated ISY1, were required for pro-miRNA biogenesis and expression of all miRNAs within the cluster except miR-92, as inhibition of either factor resulted in decreased expression of all miRNAs within the cluster except miR-92. It was further determined that SF3B1 also contributed to pro-miRNA biogenesis, as inhibition of SF3B1 also decreased expression of all miRNAs within the cluster except miR-92. Lastly it was found that an increase in the ratio of miR-17, -18a, -19a, 20a, and -19b to miR-92 from the miR-17˜92 microRNA (also known as oncomiR1), was associated with several human cancers. Accordingly, aspects of the disclosure relate to compositions and methods of modulating expression of miRNAs, e.g., modulating expression of miR-17, -18a, -19a, 20a, and/or -19b. Such compositions and methods are useful, e.g., to treat cancer and to screen for inhibitors of pro-miRNA biogenesis, such as for treatment of cancer.

In some aspects, the disclosure provides a method of treating cancer, the method comprising administering to a subject having cancer an effective amount of an inhibitor of CPSF3, ISY1, or SF3B1.

In some embodiments, the inhibitor is a small molecule, an antisense oligonucleotide, a small interfering RNA (siRNA), a microRNA (miRNA), or an antibody. In some embodiments, the inhibitor of SF3B1 is selected from the group consisting of FR901463, FR901464, FR901465, spliceostatin A (SSA), a sudemycin, a meayamycin; a pladienolide and GEX1.

In some embodiments, the cancer is a cancer associated with upregulation of oncomiR1. In some embodiments, the upregulation of oncomiR1 include upregulation of one or more of miR-17, miR-18a, miR-19a, miR-20a, or miR-19b.

Other aspects of the disclosure relate to a method of screening for an inhibitor of microRNA (miRNA) biogenesis, the method comprising contacting a cell expressing a primary microRNA 17˜92 (pri-miR-17˜92) with a candidate substance, measuring a ratio of the level of miR-17, miR-18a, miR-19a, miR-20a, and/or miR-19b to the level of miR-92; and identifying the candidate substance as an inhibitor of miRNA biogenesis if the ratio is decreased compared to a control ratio. In some embodiments, the measuring comprises a luciferase assay. In some embodiments, the luciferase assay comprises use of a Renilla Luciferase gene, wherein a 3′UTR of the Renilla Luciferase gene contains a pri-miR-17˜92, or a fragment thereof. In some embodiments, the control ratio is the ratio in a cell that has not been contacted with the candidate substance. In some embodiments, the candidate substance is a small molecule.

Yet other aspects of the disclosure relate to a variant primary microRNA (pri-miRNA) that is incapable of forming a progenitor-microRNA (pro-miRNA). In some embodiments, the variant pri-miRNA is not processed by CPSF3. In some embodiments, the variant pri-miRNA comprises a mutation in a CPSF3 cleavage domain. In some embodiments, the variant pri-miRNA comprises a mutation in the sequence CAGUCAGAAUAAUGU. In some embodiments, the mutation is a mutation in the second A and/or the second C in the sequence CAGUCAGAAUAAUGU. In some embodiments, the variant pri-miRNA is a variant pri-miR-17˜92. Other aspects of the disclosure relate to a vector comprising a coding sequence encoding a variant pri-miRNA as described above or otherwise described herein.

In other aspects, the disclosure provides a method of treating cancer in a subject, the method comprising administering to the subject an effective amount of an agent that inhibits formation of a progenitor-microRNA (pro-miRNA). In some embodiments, the agent is an inhibitor of CPSF3, ISY1, or SF3B1.

Another aspect of the disclosure relates to a method of reducing progenitor-microRNA (pro-miRNA) levels in a cell, the method comprising contacting the cell with an agent that inhibits formation of a progenitor-microRNA (pro-miRNA). In some embodiments, contacting the cell with the agent reduces the levels of one or more of miR-17, miR-18a, miR-19a, miR-20a, or miR-19b in the cell. In some embodiments, the agent is an inhibitor of CPSF3, ISY1, or SF3B1. In some embodiments, the cell is a cancer cell.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1. Posttranscriptional regulation of miR-17˜92 and identification of pro-miRNA. (A) q.RT-PCR analysis of miRNA and pri-miRNA expression in mouse ESCs over a differentiation time course of days in culture after withdrawal of Leukemia inhibitory factor (Lif) from the media. Data are normalized to snoR142 (for miRNAs) and ACTIN (for pri-miRNA) and represented as mean +/− SEM. *p <0.05, **p <0.01, Student's t test. (B) Northern blot analysis of the RNAs from (A) using probes to detect the indicated miRNAs. U6 was used as control. (C) Relative read number of small RNA sequences mapping to the indicated miRNAs in ESCs. RPM: reads per million. (D) Mapping of RNA-seq cDNA sequence to the mouse miR-17˜92 locus. cDNAs were prepared and sequenced from wt, Dgcr8−/−, and Dicer−/− ESCs. Exon-exon boundaries identified in the sequencing data are shown (bottom), and read numbers are shown in red font. Tophat (ccb.jhu.edu/software/tophat/index.shtml) was used for the analysis (E) Schematic representation of the mouse miR-17˜92 locus. P1, P2, P3, and P4 indicate the positions of the probes used for Northern blots in (F). RP1, and RP2, indicate the position of the primers used for the 5′ RACE experiments presented in (G). A zoomed in sequence of exon 3 (shaded gray) includes the position of the cleavage site identified by 5′ RACE (highlighted in green font) and the miR-17-5p sequence (highlighted in red font). (F) Northern blots performed on total RNA, PolyA+ RNA, and PolyA− RNA samples prepared from wt, Dgcr8−/−, and Dicer−/− ESCs. The probes used are indicated on the left and the interpretation of the observed bands shown in schematic format on the right. (G) 5′ RACE data from Dicer−/− ESCs using the indicated primers. (Left) shows ethidium bromide stained agarose gels loaded with the 5′ RACE PCR products and (right) shows a summary of the sequencing data with the corresponding RNA 5′ ends mapped. The numbers indicate the proportion of all sequences that map to a particular nucleotide position. Mature miRNA sequences are highlighted in red and miR-17-3p and miR-92a-1* highlighted in blue.

FIG. 2. Cleavage of pri-miR-17˜92 to pro-miRNA is a key step in miRNA maturation. (A) Microprocessor cleavage assays performed using the indicated in vitro transcribed, radiolabeled RNA substrates. Asterisk denotes a truncated or non-specific RNA. (B) q.RT-PCR analysis of the relative expression of regions of miR-17˜92 expressed from the indicated rescue plasmid. Primers amplifying the 5′ upstream sequence (5′) and primers spanning the cleavage site (CS) were used to detect pri-miR-17˜92 expressed from the indicated transgene in transfected miR-17˜92−/− ESCs. Data are normalized to ACTIN and represented as mean +/− SEM. **p <0.01, Student's t test. (C) Northern blot analysis of the RNA samples in (B) using a probe that detects the 5′ upstream region of pri-miR-17˜92 (P2). (D) miR-17˜92−/− ESCs were transfected with the indicated rescue plasmids and mature miRNAs measured by q.RT-PCR. Data are normalized to snoR142 and represented as mean +/− SEM. **p <0.01, Student's t test. (E) Northern blot analysis of the RNAs from (D) using probes to detect the indicated miRNAs. (F) q.RT-PCR analysis of the indicated endogenous mature miRNAs expression in ESCs engineered with a mutation in the cleavage site at the endogenous pri-miR-17˜92 locus. Data are normalized to snoR142 and represented as mean +/− SEM.

FIG. 3. Identification of two complementary repression domains controlling miRNA biogenesis. (A-B) Genetic rescue experiments in which miR-17˜92−/− ESCs were transfected with the indicated rescue plasmids and mature miRNAs measured by q.RT-PCR. The ˜40 nt repression domain (RD) is highlighted with blue shading in (A). Data are normalized to snoR142 and represented as mean +/− SEM. **p <0.01, Student's t test. (C) In vitro Microprocessor cleavage assays performed using the indicated non-radiolabeled substrate RNAs. Aliquots of the reaction products were loaded onto multiple gels, transferred to nylon membranes, and Northern blots performed using the indicated probes for individual pre-miRNA detection. (D) Secondary structure prediction of the minimal pri-miRNA fragment containing the 5′ repression domain (RD) using the RNAFold algorithm (rna.tbi.univie.ac.at/cgi-bin/RNAfold.cgi). The region (termed RD*—Repression Domain Star) that is complementary to the RD is depicted. (E) A zoomed in view of the base-pairing region of RD and RD*. Inset shows the in vitro annealing and native PAGE analysis of synthetic RNAs corresponding to the RD and RD* sequence. (F) Genetic rescue experiments in which miR-17˜92−/− ESCs were transfected with the indicated rescue plasmids and mature miRNAs measured by q.RT-PCR. Data are normalized to snoR142 and represented as mean +/− SEM. *p <0.05, **p <0.01, Student's t test. (G) Microprocessor cleavage assays performed using the indicated in vitro transcribed, radiolabeled RNA substrates. (H) As in (B and F) but using plasmids with the indicated nucleotide substitutions (red font).

FIG. 4. Pri-miR-17˜92 adopts an RNA conformation that inhibits Microprocessor. (A) Microprocessor cleavage assays performed using the indicated non-radiolabeled substrate RNAs with (+) or without (−) RNA annealing in the presence of MgCl2. Aliquots of the reaction products were loaded onto multiple gels, transferred to nylon membranes, and Northern blots performed using the indicated probes for individual pre-miRNA detection. (B) RNAse T1 accessibility assays performed using the indicated RNA and analyzed by reverse transcriptase primer extension using the indicated 5′-end labeled primers. (C) Negative-stain micrographs of indicated RNAs in the presence of MgCl2. Specimens were prepared in uranyl acetate. Lower panel shows representative images of RD-Pro-RD* particles. (D) 2D distribution of RD-Pro-RD* particles based on their diameter and circularities.

FIG. 5. CPSF3 endonuclease is required for pro-miRNA biogenesis and mature miRNA expression. (A) Summary of mass spec results identifying proteins that were found in each of the indicated RNA-affinity purifications. Factors known to be involved in pre-mRNA 3′ cleavage and polyadenylation are highlighted in red and proteins involved in splicing are listed in blue. (B) Western blot of lysates prepared from ESCs transfected with the siRNAs and analyzed using the indicated antibodies. (C) q.RT-PCR analysis of pri-miRNA expression in cells with indicated siRNA knockdown. Data are normalized to ACTIN and represented as mean +/− SEM. (D) q.RT-PCR analysis of the indicated endogenous miRNAs in ESCs transfected with the siRNAs shown. Data are normalized to snoR142 and represented as mean +/− SEM. (E) Northern blot performed on total RNA, from wt, Dgcr8−/−, and Dicer−/− ESCs transfected with the indicated siRNAs. A probe was used to detect pro-miRNA as indicated on the right (P3). (F) q.RT-PCR analysis of the relative expression the indicated miRNAs expressed in miR-17˜92−/− ESCs co-transfected with the indicated rescue plasmids and siRNAs. Data are normalized to snoR142 and represented as mean +/− SEM. (G) Coomassie blue stained SDS-PAGE gel (top) and αCPSF3 western analysis (bottom) of recombinant His-CPSF3 purified from E. coli. Wild-type (WT) and a catalytic mutant CPSF3 (D75K/H76A) were produced. (H, I) CPSF cleavage assays using the indicated in vitro transcribed RNA substrate and His-CPSF3 (WT or Mutant). (J) Microprocessor cleavage assay with the indicated RNA substrate and with addition of His-CPSF3 where indicated.

FIG. 6. Spliceosome subunits are required for pro-miRNA biogenesis and miRNA expression. (A) Western blot of lysates prepared from ESCs transfected with the siRNAs and analyzed using the indicated antibodies. (B) q.RT-PCR analysis of pri-miRNA expression in cells with indicated siRNA knockdown. Data are normalized to ACTIN and represented as mean +/− SEM. (C) q.RT-PCR analysis of the indicated endogenous miRNAs in ESCs transfected with the siRNAs shown. Data are normalized to snoR142 and represented as mean +/− SEM. (D) Northern blots performed on total RNA, from wt, Dgcr8−/−, and Dicer−/− ESCs transfected with the indicated siRNAs. The probes used are indicated on the left and the bands representing the pro-miRNA (P3) and the 3′ fragment (P4) of pri-miR-17˜92 are indicated on the right. (E) q.RT-PCR analysis of the relative expression the indicated miRNAs expressed in miR-17˜92−/− ESCs co-transfected with the indicated rescue plasmids and siRNAs. Data are normalized to snoR142 and represented as mean +/− SEM. (F) Flag immunoprecipitation (Flag-IP) assays performed from cells expressing the indicated Flag-tagged cDNAs together the indicated miRNA expressing plasmids. q.RT-PCR was performed on RNAs collected from the purified complexes and the relative enrichment of the pro-miRNA signal in the IP compared with input samples is plotted for each protein. (G) Schematic representation of the wt and the cleavage mutant luciferase reporters (top). Reporter assays in 293 cells were performed in triplicate and the indicated siRNAs were co-transfected with the reporter plasmid DNA (bottom). *p <0.05, **p <0.01, versus control sample, Student's t test.

FIG. 7. Pro-miRNA biogenesis controls miR-17˜92 expression in embryonic stem cells (A) q.RT-PCR analysis of the indicated mRNA expression in mouse ESCs over a differentiation time course. Data are normalized to ACTIN and represented as mean +/− SEM. **p <0.01, Student's t test. (B) Western blot analysis of cell lysates prepared from ESCs over a differentiation time course. (C) q.RT-PCR analysis of the relative expression of regions of the endogenous pri-miR-17˜92 during ESC differentiation. Primers amplifying a region spanning the cleavage site (CS), a region in the 5′ upstream sequence (5′), and a region in the 3′ downstream sequence (3′) of pri-miR-17˜92 were used. Data are normalized to ACTIN and represented as mean +/− SEM. *p <0.05, Student's t test. (D) q.RT-PCR analysis of ectopic ISY1 expression, endogenous pri-miR-17˜92 expression using the primers as in (C) and the endogenous miRNAs indicated. Data are normalized to sno142 (for miRNAs) and ACTIN (for pri-miRNA) and represented as mean +/− SEM. *p <0.05, **p <0.01, Student's t test. (E, F) Co-immunoprecipitation (co-IP) assays performed by using the indicated Flag-tagged cDNAs, performing Flag-affinity purifications, and analyzing the affinity eluate Western blot using indicated antibodies. Where indicated lysates and IPs were treated with RNase A. (G) CPSF cleavage assays with His-CPSF3 and Flag-ISY1 complex purified from HEK293 cells. (H) Model for the posttranscriptional control of miR-17˜92 biogenesis.

FIG. 8. Cleavage of pri-miR-17˜92 to pro-miRNA is a key step in miRNA maturation. (A) miR-17˜92−/− ESCs were transfected with the indicated rescue plasmids and mature miRNAs measured by q.RT-PCR. Data are normalized to sno142 and represented as mean +/− SEM.

FIG. 9. Identification of two complementary repression domains controlling miRNA biogenesis. (A) Alignment analysis of Repression domain and Repression Domain* in different species. (B), A zoomed in view of the base-pairing region of RD and RD* of pri-miR-17˜92 in human.

FIG. 10. CPSF3 endonuclease is required miRNA biogenesis. (A, B) q.RT-PCR 2 0 analysis of mRNA expression in cells with indicated siRNA knockdown. Data are normalized to ACTIN and represented as mean +/− SEM. (C) q.RT-PCR analysis of the indicated endogenous miRNAs in ESCs transfected with the siRNAs shown. Data are normalized to snoR142 and represented as mean +/− SEM. (D) q.RT-PCR analysis of pri-miRNA expression in cells with indicated siRNA knockdown. Data are normalized to ACTIN and represented as mean +/− SEM.

FIG. 11. Certain spliceosome subunits are required for miRNA biogenesis. (A, B) q.RT-PCR analysis of mRNA expression in cells with indicated siRNA knockdown. Data are normalized to ACTIN and represented as mean +/− SEM. (C) q.RT-PCR analysis of the indicated endogenous miRNAs in ESCs transfected with the siRNAs shown. Data are normalized to snoR142 and represented as mean +/− SEM. (D) q.RT-PCR analysis of pri-miRNA expression in cells with indicated siRNA knockdown. Data are normalized to ACTIN and represented as mean +/− SEM.

FIG. 12. Pro-miRNA biogenesis controls miR-17˜92 expression in human cancer. Analysis of relative miRNA levels in primary human lung squamous cell carcinoma using data from TCGA.

FIG. 13. Pro-miRNA biogenesis controls miR-17˜92 expression in human cancer. (Top graph) q.RT-PCR analysis of the indicated genes in H1299 lung cancer cells transfected with the indicated siRNAs. Data are normalized to ACTIN and represented as mean +/− SEM. (Bottom graph) q.RT-PCR analysis of the indicated endogenous miRNAs in H1299 cells transfected with the siRNAs shown. Data are normalized to U6 RNA and represented as mean +/− SEM.

FIG. 14. Pro-miRNA biogenesis controls miR-17˜92 expression in human cancer. Analysis of relative miRNA levels in primary human colon adenocarcinoma using data from TCGA.

FIG. 15. Pro-miRNA biogenesis controls miR-17˜92 expression in human cancer. (Top graph) q.RT-PCR analysis of the indicated genes in A549 cancer cells transfected with the indicated siRNAs. Data are normalized to ACTIN and represented as mean +/− SEM. (Bottom graph) q.RT-PCR analysis of the indicated endogenous miRNAs in A549 cells transfected with the siRNAs shown. Data are normalized to U6 RNA and represented as mean +/− SEM.

FIG. 16. An exemplary annotated sequence of pri-miR-17˜92a.

### DETAILED DESCRIPTION OF THE INVENTION

Aspects of the disclosure relate to compositions and methods for modulating microRNA (miRNA) biogenesis. In some aspects, the disclosure is based, in part, on a study showing a novel intermediate in miRNA biogenesis, referred to herein as a progenitor micoRNA (pro-miRNA), which was required for proper processing of primary microRNA 17˜92 (pri-miR-17˜92) into pre-miR-17, miR-18a, miR-19a, miR-20a, and miR-19b. CPSF3 (CPSF73), and the Spliceosome-associated ISY1, and SF3B1 were all shown to contribute to pro-miRNA biogenesis, as inhibition of any one of these factors decreased expression of all miRNAs within the cluster except miR-92. Further, it was found that an increase in the ratio of miR-17, -18a, -19a, 20a, and -19b to miR-92 from the miR-17˜92 microRNA (also known as oncomiR1), was associated with several human cancers. Additionally, ISY1 knockdown in human lung cancer cell lines was shown to cause the selective decreased expression of miR-17, -19a, -19b, and -20. Accordingly, it is believed that modulation of miR-17˜92 microRNA biogenesis, such as by inhibiting CPSF3, ISY1, and/or SF3B1 may be useful, e.g., in treatment of cancer.

### Methods of Treatment

Aspects of the disclosure relate to a method of treating cancer. In some embodiments, the method comprises administering to a subject (e.g., a subject having cancer) an effective amount of an inhibitor of CPSF3, ISY1, or SF3B1. In some embodiments, the method comprises administering to a subject (e.g., a subject having cancer) an effective amount of an agent that inhibits formation of a progenitor-microRNA (pro-miR). In some embodiments, the agent is an inhibitor of CPSF3, ISY1, or SF3B1.

CPSF3 (Cleavage and polyadenylation specificity factor subunit 3) is a component of the cleavage and polyadenylation specificity factor (CPSF) complex. An exemplary human CPSF3 protein sequence is provided below.

>sp|Q9UKF6|CPSF3_HUMAN
MSAIPAEESDQLLIRPLGAGQEVGRSCIILEFKGRKIMLDCGIHPGLE
GMDALPYIDLIDPAEIDLLLISHFHLDHCGALPWFLQKTSFKGRTFMT
HATKAIYRWLLSDYVKVSNISADDMLYTETDLEESMDKIETINFHEVK
EVAGIKFWCYHAGHVLGAAMFMIEIAGVKLLYTGDFSRQEDRHLMAAE
IPNIKPDILIIESTYGTHIHEKREEREARFCNTVHDIVNRGGRGLIPV
FALGRAQELLLILDEYWQNHPELHDIPIYYASSLAKKCMAVYQTYVNA
MNDKIRKQININNPFVFKHISNLKSMDHFDDIGPSVVMASPGMMQSGL
SRELFESWCTDKRNGVIIAGYCVEGTLAKHIMSEPEEITTMSGQKLPL
KMSVDYISFSAHTDYQQTSEFIRALKPPHVILVHGEQNEMARLKAALI
REYEDNDEVHIEVHNPRNTEAVTLNFRGEKLAKVMGFLADKKPEQGQR
VSGILVKRNFNYHILSPCDLSNYTDLAMSTVKQTQAIPYTGPFNLLCY
QLQKLTGDVEELEIQEKPALKVFKNITVIQEPGMVVLEWLANPSNDMY
ADTVTTVILEVQSNPKIRKGAVQKVSKKLEMHVYSKRLEIMLQDIFGE
DCVSVKDDSILSVTVDGKTANLNLETRTVECEEGSEDDESLREMVELA
AQRLYEALTPVH

ISY1 (Pre-mRNA-splicing factor ISY1 homolog) is a protein was shown in a study herein to be involved in pro-miRNA biogenesis. An exemplary human ISY1 protein sequence is provided below.

>sp|Q9ULR0|ISY1_HUMAN
MARNAEKAMTALARFRQAQLEEGKVKERRPFLASECTELPKAEKWRRQ
IIGEISKKVAQIQNAGLGEFRIRDLNDEINKLLREKGHWEVRIKELGG
PDYGKVGPKMLDHEGKEVPGNRGYKYFGAAKDLPGVRELFEKEPLPPP
RKTRAELMKAIDFEYYGYLDEDDGVIVPLEQEYEKKLRAELVEKWKAE
REARLARGEKEEEEEEEEEINIYAVTEEESDEEGSQEKGGDDSQQKFI
AHVPVPSQQEIEEALVRRKKMELLQKYASETLQAQSEEARRLLGY

SF3B1 (Splicing factor 3B subunit 1) is a subunit of the splicing factor SF3B required for A complex assembly. An exemplary human SF3B1 protein sequence is provided below.

>sp|O75533|SF3B1_HUMAN
MAKIAKTHEDIEAQIREIQGKKAALDEAQGVGLDSTGYYDQEIYGGSD
SRFAGYVTSIAATELEDDDDDYSSSTSLLGQKKPGYHAPVALLNDIPQ
STEQYDPFAEHRPPKIADREDEYKKHRRTMIISPERLDPFADGGKTPD
PKMNARTYMDVMREQHLTKEEREIRQQLAEKAKAGELKVVNGAAASQP
PSKRKRRWDQTADQTPGATPKKLSSWDQAETPGHTPSLRWDETPGRAK
GSETPGATPGSKIWDPTPSHTPAGAATPGRGDTPGHATPGHGGATSSA
RKNRWDETPKTERDTPGHGSGWAETPRTDRGGDSIGETPTPGASKRKS
RWDETPASQMGGSTPVLTPGKTPIGTPAMNMATPTPGHIMSMTPEQLQ
AWRWEREIDERNRPLSDEELDAMFPEGYKVLPPPAGYVPIRTPARKLT
ATPTPLGGMTGFHMQTEDRTMKSVNDQPSGNLPFLKPDDIQYFDKLLV
DVDESTLSPEEQKERKIMKLLLKIKNGTPPMRKAALRQITDKAREFGA
GPLFNQILPLLMSPTLEDQERHLLVKVIDRILYKLDDLVRPYVHKILV
VIEPLLIDEDYYARVEGREIISNLAKAAGLATMISTMRPDIDNMDEYV
RNTTARAFAVVASALGIPSLLPFLKAVCKSKKSWQARHTGIKIVQQIA
ILMGCAILPHLRSLVEIIEHGLVDEQQKVRTISALAIAALAEAATPYG
IESFDSVLKPLWKGIRQHRGKGLAAFLKAIGYLIPLMDAEYANYYTRE
VMLILIREFQSPDEEMKKIVLKVVKQCCGTDGVEANYIKTEILPPFFK
HFWQHRMALDRRNYRQLVDTTVELANKVGAAEIISRIVDDLKDEAEQY
RKMVMETIEKIMGNLGAADIDHKLEEQLIDGILYAFQEQTTEDSVMLN
GFGTVVNALGKRVKPYLPQICGTVLWRLNNKSAKVRQQAADLISRTAV
VMKTCQEEKLMGHLGVVLYEYLGEEYPEVLGSILGALKAIVNVIGMHK
MTPPIKDLLPRLTPILKNRHEKVQENCIDLVGRIADRGAEYVSAREWM
RICFELLELLKAHKKAIRRATVNTFGYIAKAIGPHDVLATLLNNLKVQ
ERQNRVCTTVAIAIVAETCSPFTVLPALMNEYRVPELNVQNGVLKSLS
FLFEYIGEMGKDYIYAVTPLLEDALMDRDLVHRQTASAVVQHMSLGVY
GFGCEDSLNHLLNYVWPNVFETSPHVIQAVMGALEGLRVAIGPCRMLQ
YCLQGLFHPARKVRDVYWKIYNSIYIGSQDALIAHYPRIYNDDKNTYI
RYELDYIL

As used herein, “treat” or “treatment” of cancer includes, but is not limited to, preventing, reducing, or halting the development of a cancer, reducing or eliminating the symptoms of cancer, suppressing or inhibiting the growth of a cancer, preventing or reducing metastasis and/or invasion of an existing cancer, promoting or inducing regression of the cancer, inhibiting or suppressing the proliferation of cancerous cells, reducing angiogenesis and/or increasing the amount of apoptotic cancer cells.

The subject may be any subject, such as a human subject having cancer. Any type of cancer is contemplated herein, including, but not limited to, leukemias, lymphomas, myelomas, carcinomas, metastatic carcinomas, sarcomas, adenomas, nervous system cancers and genitourinary cancers. Exemplary cancer types include adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, osteosarcoma, fibrous histiocytoma, brain cancer, brain stem glioma, cerebellar astrocytoma, malignant glioma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, extracranial germ cell tumor, extragonadal germ cell tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, intraocular melanoma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinema, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, or Wilms tumor. Subjects having cancer may be identified using any method known in the art (e.g., blood tests, histology, CT scan, X-ray, MRI, physical exam, cytogenitic analysis, urinalysis, or genetic testing). A subject suspected of having cancer might show one or more symptoms of the disease. Signs and symptoms for cancer are well known to those of ordinary skill in the art.

In some embodiments of any one of the methods, the subject has a cancer that is associated with upregulation of oncomiR1. In some embodiments, upregulation of oncomiR1 including upregulation of one or more of miR-17, miR-18a, miR-19a, miR-20a, or miR-19b. As used herein, “upregulation of oncomiR1 or upregulation of one or more of miR-17, miR-18a, miR-19a, miR-20a, or miR-19b” means that the level of oncomiR1 or of one or more of miR-17, miR-18a, miR-19a, miR-20a, or miR-19b is above a control level, such as a pre-determined threshold or a level in a control sample. In some embodiments, the control sample is a cell, tissue or fluid obtained from a healthy subject or population of healthy subjects. As used herein, a healthy subject is a subject that is apparently free of disease and has no history of disease, such as cancer. In some embodiments, the control sample is obtained from a subject having cancer, such as a non-cancerous cell or tissue obtained from the subject having the cancer. In some embodiments, a control level is a level that is undetectable or below a background/noise level obtained using standard methods of detection (e.g., Western blot or immunohistochemistry). Upregulation includes a level that is, for example, 1%, 5%, 10%, 20%, 30%, 40%, 50%, 60%, 70%, 80%, 90%, 100%, 150%, 200%, 300%, 400%, 500% or more above a control level.

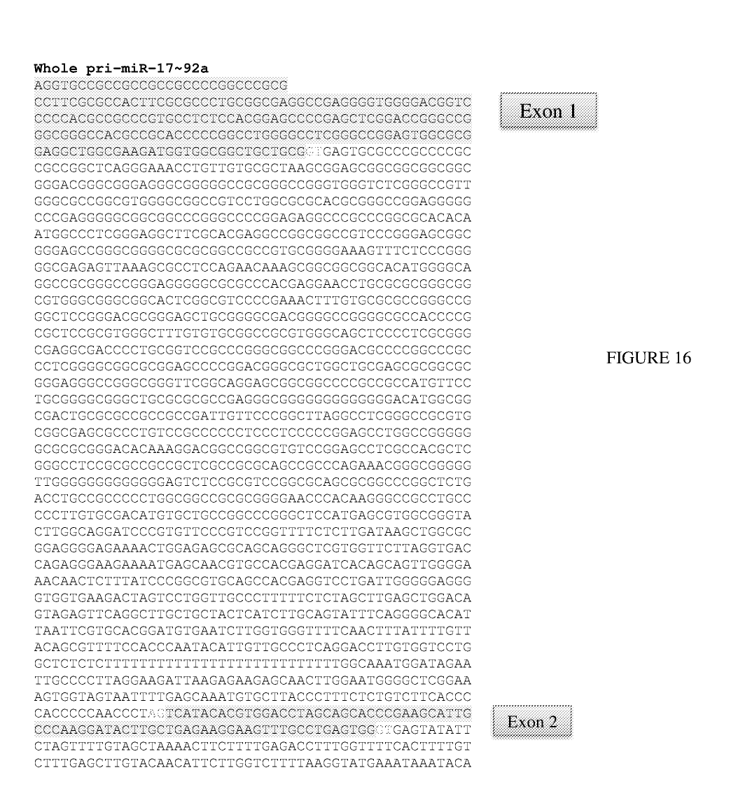

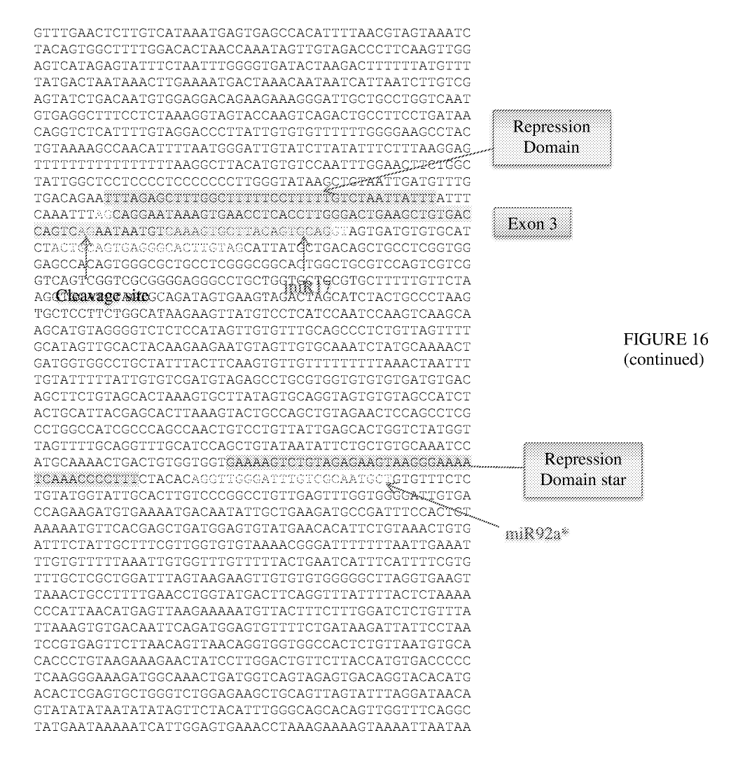

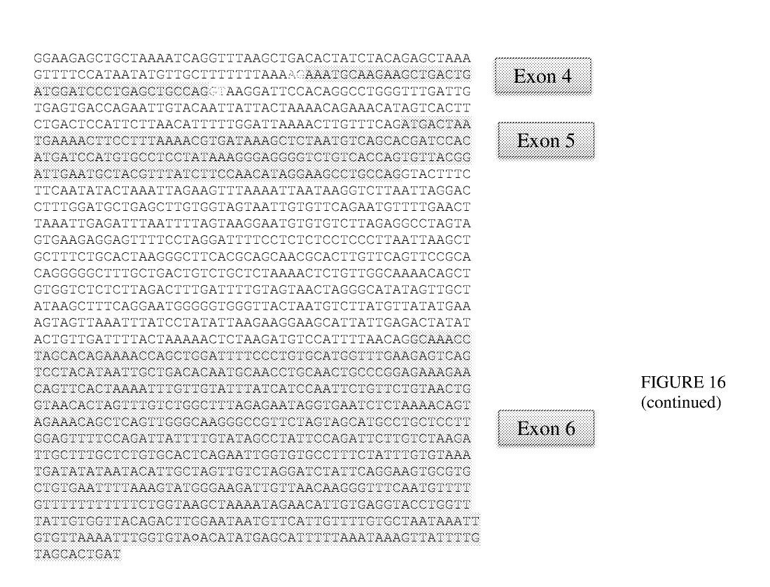

Exemplary, non-limiting sequences of pri-miR-17˜92, pro-miR, pre-miR-17, pre-18A, pre-19A, pre-20A, pre-19B, and pre-92 are provided below and in FIG. 16.

pri-miR-17~92a
AGGTGCCGCCGCCGCCGCCCCGGCCCGCGCCTTCGCGCCACTTCGCGC
CCTGCGGCGAGGCCGAGGGGTGGGGACGGTCCCCCACGCCGCCCGTGC
CTCTCCACGGAGCCCCGAGCTCGGACCGGGCCGGGCGGGCCACGCCGC
ACCCCCGGCCTGGGGCCTCGGGCCGGAGTGGCGCGGAGGCTGGCGAAG
ATGGTGGCGGCTGCTGCGGTGAGTGCGCCCGCCCCGCCGCCGGCTCAG
GGAAACCTGTTGTGCGCTAAGCGGAGCGGCGGCGGCGGCGGGACGGGC
GGGAGGGCGGGGGCCGCGGGCCGGGTGGGTCTCGGGCCGTTGGGCGCC
GGCGTGGGGCGGCCGTCCTGGCGCGCACGCGGGCCGGAGGGGGCCCGA
GGGGGCGGCGGCCCGGGCCCCGGAGAGGCCCGCCCGGCGCACACAATG
GCCCTCGGGAGGCTTCGCACGAGGCCGGCGGCCGTCCCGGGAGCGGCG
GGAGCCGGGCGGGGCGCGCGGCCGCCGTGCGGGGAAAGTTTCTCCCGG
GGGCGAGAGTTAAAGCGCCTCCAGAACAAAGCGGCGGCGGCACATGGG
GCAGGCCGCGGGCCGGGAGGGGGCGCGCCCACGAGGAACCTGCGCGCG
GGCGGCGTGGGCGGGCGGCACTCGGCGTCCCCGAAACTTTGTGCGCGC
CGGGCCGGGCTCCGGGACGCGGGAGCTGCGGGGCGACGGGGCCGGGGC
GCCACCCCGCGCTCCGCGTGGGCTTTGTGTGCGGCCGCGTGGGCAGCT
CCCCTCGCGGGCGAGGCGACCCCTGCGGTCCGCCCGGGCGGCCCGGGA
CGCCCCGGCCCGCCCTCGGGGCGGCGCGGAGCCCCGGACGGGCGCTGG
CTGCGAGCGCGGCGCGGGAGGGCCGGGCGGGTTCGGCAGGAGCGGCGG
CCCCGCCGCCATGTTCCTGCGGGGCGGGCTGCGCGCGCCGAGGGCGGG
GGGGGGGGGGACATGGCGGCGACTGCGCGCCGCCGCCGATTGTTCCCG
GCTTAGGCCTCGGGCCGCGTGCGGCGAGCGCCCTGTCCGCCCCCCTCC
CTCCCCCGGAGCCTGGCCGGGGGGCGCGCGGGACACAAAGGACGGCCG
GCGTGTCCGGAGCCTCGCCACGCTCGGGCCTCCGCGCCGCCGCTCGCC
GCGCAGCCGCCCAGAAACGGGCGGGGGTTGGGGGGGGGGGGGAGTCTC
CGCGTCCGGCGCAGCGCGGCCCGGCTCTGACCTGCCGCCCCCTGGCGG
CCGCGCGGGGAACCCACAAGGGCCGCCTGCCCCCTTGTGCGACATGTG
CTGCCGGCCCGGGCTCCATGAGCGTGGCGGGTACTTGGCAGGATCCCG
TGTTCCCGTCCGGTTTTCTCTTGATAAGCTGGCGCGGAGGGGAGAAAA
CTGGAGAGCGCAGCAGGGCTCGTGGTTCTTAGGTGACCAGAGGGAAGA
AAATGAGCAACGTGCCACGAGGATCACAGCAGTTGGGGAAACAACTCT
TTATCCCGGCGTGCAGCCACGAGGTCCTGATTGGGGGAGGGGTGGTGA
AGACTAGTCCTGGTTGCCCTTTTTCTCTAGCTTGAGCTGGACAGTAGA
GTTCAGGCTTGCTGCTACTCATCTTGCAGTATTTCAGGGGCACATTAA
TTCGTGCACGGATGTGAATCTTGGTGGGTTTTCAACTTTATTTTGTTA
CAGCGTTTTCCACCCAATACATTGTTGCCCTCAGGACCTTGTGGTCCT
GGCTCTCTCTTTTTTTTTTTTTTTTTTTTTTTTTTTGGCAAATGGATA
GAATTGCCCCTTAGGAAGATTAAGAGAAGAGCAACTTGGAATGGGGCT
CGGAAAGTGGTAGTAATTTTGAGCAAATGTGCTTACCCTTTCTCTGTC
TTCACCCCACCCCCAACCCTAGTCATACACGTGGACCTAGCAGCACCC
GAAGCATTGCCCAAGGATACTTGCTGAGAAGGAAGTTTGCCTGAGTGG
GTGAGTATATTCTAGTTTTGTAGCTAAAACTTCTTTTGAGACCTTTGG
TTTTCACTTTTGTCTTTGAGCTTGTACAACATTCTTGGTCTTTTAAGG
TATGAAATAAATACAGTTTGAACTCTTGTCATAAATGAGTGAGCCACA
TTTTAACGTAGTAAATCTACAGTGGCTTTTGGACACTAACCAAATAGT
TGTAGACCCTTCAAGTTGGAGTCATAGAGTATTTCTAATTTGGGGTGA
TACTAAGACTTTTTTATGTTTTATGACTAATAAACTTGAAAATGACTA
AACAATAATCATTAATCTTGTCGAGTATCTGACAATGTGGAGGACAGA
AGAAAGGGATTGCTGCCTGGTCAATGTGAGGCTTTCCTCTAAAGGTAG
TACCAAGTCAGACTGCCTTCCTGATAACAGGTCTCATTTTGTAGGACC
CTTATTGTGTGTTTTTTGGGGAAGCCTACTGTAAAAGCCAACATTTTA
ATGGGATTGTATCTTATATTTCTTTAAGGAGTTTTTTTTTTTTTTTTA
AGGCTTACATGTGTCCAATTTGGAACTTCTGGCTATTGGCTCCTCCCC
TCCCCCCCTTGGGTATAAGCTGTAATTGATGTTTGTGACAGAATTTAG
AGCTTTGGCTTTTTCCTTTTTGTCTAATTATTTATTTCAAATTTAGCA
GGAATAAAGTGAACCTCACCTTGGGACTGAAGCTGTGACCAGTCAGAA
TAATGTCAAAGTGCTTACAGTGCAGGTAGTGATGTGTGCATCTACTGC
AGTGAGGGCACTTGTAGCATTATGCTGACAGCTGCCTCGGTGGGAGCC
ACAGTGGGCGCTGCCTCGGGCGGCACTGGCTGCGTCCAGTCGTCGGTC
AGTCGGTCGCGGGGAGGGCCTGCTGGTGCTGCGTGCTTTTTGTTCTAA
GGTGCATCTAGTGCAGATAGTGAAGTAGACTAGCATCTACTGCCCTAA
GTGCTCCTTCTGGCATAAGAAGTTATGTCCTCATCCAATCCAAGTCAA
GCAAGCATGTAGGGGTCTCTCCATAGTTGTGTTTGCAGCCCTCTGTTA
GTTTTGCATAGTTGCACTACAAGAAGAATGTAGTTGTGCAAATCTATG
CAAAACTGATGGTGGCCTGCTATTTACTTCAAGTGTTGTTTTTTTTTA
AACTAATTTTGTATTTTTATTGTGTCGATGTAGAGCCTGCGTGGTGTG
TGTGATGTGACAGCTTCTGTAGCACTAAAGTGCTTATAGTGCAGGTAG
TGTGTAGCCATCTACTGCATTACGAGCACTTAAAGTACTGCCAGCTGT
AGAACTCCAGCCTCGCCTGGCCATCGCCCAGCCAACTGTCCTGTTATT
GAGCACTGGTCTATGGTTAGTTTTGCAGGTTTGCATCCAGCTGTATAA
TATTCTGCTGTGCAAATCCATGCAAAACTGACTGTGGTGGTGAAAAGT
CTGTAGAGAAGTAAGGGAAAATCAAACCCCTTTCTACACAGGTTGGGA
TTTGTCGCAATGCTGTGTTTCTCTGTATGGTATTGCACTTGTCCCGGC
CTGTTGAGTTTGGTGGGGATTGTGACCAGAAGATGTGAAAATGACAAT
ATTGCTGAAGATGCCGATTTCCACTGTAAAAATGTTCACGAGCTGATG
GAGTGTATGAACACATTCTGTAAACTGTGATTTCTATTGCTTTCGTTG
GTGTGTAAAACGGGATTTTTTTAATTGAAATTTGTGTTTTTAAATTGT
GGTTTGTTTTTACTGAATCATTTCATTTTCGTGTTTGCTCGCTGGATT
TAGTAAGAAGTTGTGTGTGGGGGCTTAGGTGAAGTTAAACTGCCTTTT
GAACCTGGTATGACTTCAGGTTTATTTTACTCTAAAACCCATTAACAT
GAGTTAAGAAAAATGTTACTTTCTTTGGATCTCTGTTTATTAAAGTGT
GACAATTCAGATGGAGTGTTTTCTGATAAGATTATTCCTAATCCGTGA
GTTCTTAACAGTTAACAGGTGGTGGCCACTCTGTTAATGTGCACACCC
TGTAAGAAAGAACTATCCTTGGACTGTTCTTACCATGTGACCCCCTCA
AGGGAAAGATGGCAAACTGATGGTCAGTAGAGTGACAGGTACACATGA
CACTCGAGTGCTGGGTCTGGAGAAGCTGCAGTTAGTATTTAGGATAAC
AGTATATATAATATATAGTTCTACATTTGGGCAGCACAGTTGGTTTCA
GGCTATGAATAAAAATCATTGGAGTGAAACCTAAAGAAAAGTAAAATT
AATAAGGAAGAGCTGCTAAAATCAGGTTTAAGCTGACACTATCTACAG
AGCTAAAGTTTTCCATAATATGTTGCTTTTTTTAAAAGAAATGCAAGA
AGCTGACTGATGGATCCCTGAGCTGCCAGGTAAGGATTCCACAGGCCT
GGGTTTGATTGTGAGTGACCAGAATTGTACAATTATTACTAAAACAGA
AACATAGTCACTTCTGACTCCATTCTTAACATTTTTGGATTAAAACTT
GTTTCAGATGACTAATGAAAACTTCCTTTAAAACGTGATAAAGCTCTA
ATGTCAGCACGATCCACATGATCCATGTGCCTCCTATAAAGGGAGGGG
TCTGTCACCAGTGTTACGGATTGAATGCTACGTTTATCTTCCAACATA
GGAAGCCTGCCAGGTACTTTCTTCAATATACTAAATTAGAAGTTTAAA
ATTAATAAGGTCTTAATTAGGACCTTTGGATGCTGAGCTTGTGGTAGT
AATTGTGTTCAGAATGTTTTGAACTTAAATTGAGATTTAATTTTAGTA
AGGAATGTGTGTCTTAGAGGCCTAGTAGTGAAGAGGAGTTTTCCTAGG
ATTTTCCTCTCTCCTCCCTTAATTAAGCTGCTTTCTGCACTAAGGGCT
TCACGCAGCAACGCACTTGTTCAGTTCCGCACAGGGGGCTTTGCTGAC
TGTCTGCTCTAAAACTCTGTTGGCAAAACAGCTGTGGTCTCTCTTAGA
CTTTGATTTTGTAGTAACTAGGGCATATAGTTGCTATAAGCTTTCAGG
AATGGGGGTGGGTTACTAATGTCTTATGTTATATGAAAGTAGTTAAAT
TTATCCTATATTAAGAAGGAAGCATTATTGAGACTATATACTGTTGAT
TTTACTAAAAACTCTAAGATGTCCATTTTAACAGGCAAACCTAGCACA
GAAAACCAGCTGGATTTTCCCTGTGCATGGTTTGAAGAGTCAGTCCTA
CATAATTGCTGACACAATGCAACCTGCAACTGCCCGGAGAAAGAACAG
TTCACTAAAATTTGTTGTATTTATCATCCAATTCTGTTCTGTAACTGG
TAACACTAGTTTGTCTGGCTTTAGAGAATAGGTGAATCTCTAAAACAG
TAGAAACAGCTCAGTTGGGCAAGGGCCGTTCTAGTAGCATGCCTGCTC
CTTGGAGTTTTCCAGATTATTTTGTATAGCCTATTCCAGATTCTTGTC
TAAGATTGCTTTGCTCTGTGCACTCAGAATTGGTGTGCCTTTCTATTT
GTGTAAATGATATATAATACATTGCTAGTTGTCTAGGATCTATTCAGG
AAGTGCGTGCTGTGAATTTTAAAGTATGGGAAGATTGTTAACAAGGGT
TTCAATGTTTTGTTTTTTTTTTTCTGGTAAGCTAAAATAGAACATTGT
GAGGTACCTGGTTTATTGTGGTTACAGACTTGGAATAATGTTCATTGT
TTTGTGCTAATAAATTGTGTTAAAATTTGGTGTA∘ACATATGAGCATT
TTTAAATAAAGTTATTTTGTAGCACTGAT
Pro-miRNA
GAATAATGTCAAAGTGCTTACAGTGCAGGTAGTGATGTGTGCAT
CTACTGCAGTGAGGGCACTTGTAGCATTATGCTGACAGCTGCCTCGGTGG
GAGCCACAGTGGGCGCTGCCTCGGGCGGCACTGGCTGCGTCCAGTCGTCG
GTCAGTCGGTCGCGGGGAGGGCCTGCTGGTGCTGCGTGCTTTTTGTTCTA
AGGTGCATCTAGTGCAGATAGTGAAGTAGACTAGCATCTACTGCCCTAAG
TGCTCCTTCTGGCATAAGAAGTTATGTCCTCATCCAATCCAAGTCAAGCA
AGCATGTAGGGGTCTCTCCATAGTTGTGTTTGCAGCCCTCTGTTAGTTTT
GCATAGTTGCACTACAAGAAGAATGTAGTTGTGCAAATCTATGCAAAACT
GATGGTGGCCTGCTATTTACTTCAAGTGTTGTTTTTTTTTAAACTAATTT
TGTATTTTTATTGTGTCGATGTAGAGCCTGCGTGGTGTGTGTGATGTGAC
AGCTTCTGTAGCACTAAAGTGCTTATAGTGCAGGTAGTGTGTAGCCATCT
ACTGCATTACGAGCACTTAAAGTACTGCCAGCTGTAGAACTCCAGCCTCG
CCTGGCCATCGCCCAGCCAACTGTCCTGTTATTGAGCACTGGTCTATGGT
TAGTTTTGCAGGTTTGCATCCAGCTGTATAATATTCTGCTGTGCAAATCC
ATGCAAAACTGACTGTGGTGGTGAAAAGTCTGTAGAGAAGTAAGGGAAAA
TCAAACCCCTTTCTACAC
Human pri-miRNA
>hg19_dna range = chr13:92000074-92006829
5′pad = 0 3′pad = 0 strand = +
repeatMasking = none
GAAGCTCTCCTCGCGGGGCGGGCCGGCCGGCCGCACCCCCGGCCTGGGGC
CTCCGGTCGTAGTAAAGCGCAGGCGGGCGGGGAGGCGGGAGCAGGAGCCC
GCGGCCGGCCAGCCGAAGATGGTGGCGGCTACTCCTCCTGGTGAGTCTGC
CCGCCCCTCCGGCGACGGAGGGAAACCTGTTGTGTGCGGCCCGGGTCTGG
CGGGCGGGGCGGAGCGGCCCGGGGCGGACTGGCCCGGGGCAGCGTGGCGG
CGGCGGCGTGGCCGGGGCGGGTCTCGGCCGTTGGCCGCCCCGGCGTGTGG
CAGCCGCATCTGGCTGCCCCCTCGCTCGCCCGCGGGCCGGCGGAGGGGGG
CAGGGCCGGGGCGGGAGGGTGGGAGGGGGCGGCGTGCGCGTGGCGGCCGC
GCCCGGGACCCGCGCAGACCCTGCCTGGGCCGACCCGAAGGCGGGTGGGC
GGACGGCGAACACAATGGCCCCTCGGGGAGAGGACGTGCGAGGCCCGTGC
CTTCTCCGGGGCCCGGGGCGCGCGCGGGGCGTGGGGTCTCTGGGTAGGAA
AGTTTCTCCCGAGGGCGAGAGTTAAAGCGCCTCCAGAACAAAGCGGCGGC
GGCGGCGGCACATGGGGCAGGCCGCGGGCCGGGAGGGGGCGCGCCCACGA
GGTACCTGCGCGCCAGCGGGCGGCGTGGCGTGGGCGGGAGCCCGCGGTTC
CCCAAACTTTGTACGCGCGAGGGTGGGCGGAGGGGCGCCGAGATCGGCGC
GGCCTGGGCGCCACCCCCGCTCCGCGTGGGCTTTGTTAGCCCGCGTGGGC
AGCCTCGGGGCGGGGCCCGCAACTTCCCCGCCGTGGCCCTCGGAGGAGGC
CGCAGTCGGCCTCAGCCGCGGCGTGGAGCCGCCTGCGCCCGGCCGCTTGC
TGGGAGTGTGGCGCGGGAGGGCCAGCCCGGCTCGGCGGGAGCGGCGTCCC
CGCCGCCATGTTCCTGCGGGGCGGGCTGCACGGGGGTGAGGGCGGGGGAC
ATGGCGGCGACTGCGCGCCGCCGCCGATTGTTCCCGGCTTAGGCCTCGGG
CCGCGTGCGACGGGCACCGCGGCCGCGGAGCCCCCGCCCCTCTGGGCCGG
GCTCGGGGGGGCTGGGGGACACAAAGGAGGGGCGGCGCGCCCGCGTCCCC
GCCGCACTCGGGCCTCGGCGCCGCCGGTCGCCGCGCGGCTGCCGCCGGGA
AACGGGTTGGGGGGGTTGCCGCGTCCGGCGGGGCCTGACTCTGACCCGCC
GCCCCCTGGCGGCTACGCGGAGAATCGCAGGGCCGCGCTCCCCCTTGTGC
GACATGTGCTGCCGGCCCGGGCTCCATGAGCGTGGCGGGCACTTTGCAGT
CTCGGGTGTTCCTGCCCGGTCTTCTGTTCCTAAACTGCAGCAAAGGGAAA
AGGAACTGAAAAAGGCAGGCTCGTCGTTGCAATATCACCAAAAGAGAAAA
TTAACGGCATGCCATCAGGACCACAGCAGTTGGAGAAACAACTCTTTATC
CCGGCTTGCAGCCACGAGGTCTTGATTGGGGGAGGGGTGGTGAAGAATAG
TCTGTGGGCTGCTTTTTTTTTTTCCTTTTACTGGAGCTGTACAGTGGAGT
CGGTGATTGCTGCTGATCATAATCAAGTATTTTAGGAGCTTATTTAGACA
TGTATCTGATAGCTAAGGATTTTTCAACTTTATTCTCTTACGTATTTTTC
AACTGTAAATTATTGGGCTTTTAAATCCTGCTAGTATTGCTCGACTCTTA
CTCTCACAAATGGATGGAATTAATTGCTGTTAGGAGGTTGGAAAATAGCA
AATATAGATTTGGACGGTGGTAGTAATTTTGAGCAAATAATGTTTTATCT
TTTTTTTCCTTATTTTTCCCTATTCCAGTCATACACGTGGACCTAACTGC
ACCAGTAGCTTTTCTGAGAATACTTGCTGAAAAGGAAGTTTTCTGGAATG
GGTAAGTGTATTCTGATTTTCTTGAACTTTTCTTAAAAACAAATTTTTCT
TGCTATTAAAGTTGAATAAATAGGATTGGTTTCTTAGAGAGTAAAAGTAG
GTGTTTCTTTCTTTAGACAATGTACCTTTTCTGAAAAACTAACTCATTAA
GTACGGATTTGCTAATTTTAAGGTAGTAAAATTACAGTGTAAATATTCCT
GTACATTTTTGGAAACTGGCTTATGCAGTTTACGAAATATAATTTTAGAC
CCTCTTTTAAGTTGGGTGATAAAGTAGATATAACCTGAGATGATAGATTT
AAACAGGATATTTACGTTCTGCTACAATTGACTGATAACACTTGAAGTGT
AGTCTGAACAGTAATTTTGTTAATCATTTCAACAAGTATTTGCTAAGTGG
AAGCCAGAAGAGGAGGAAAATGTTTTGCCACGTGGATGTGAAGATTTCCT
CTAAAAGGTACACATGGACTAAATTGCCTTTAAATGTTCCAAAATTAGTT
CTCATTTATTTGCAGTCTCATTTTGTTTTGTTTTTTTTCTCTATGTGTCA
ATCCATTTGGGAGAGGCCAGCCATTGGAAGAGCCACCACTTCCAGTGCTA
GTTGGATGGTTGGTTATGATTGCCTTCTGTAAAGAATTCTTAAGGCATAA
ATACGTGTCTAAATGGACCTCATATCTTTGAGATAATTAAACTAATTTTT
TCTTCCCCATTAGGGATTATGCTGAATTTGTATGGTTTATAGTTGTTAGA
GTTTGAGGTGTTAATTCTAATTATCTATTTCAAATTTAGCAGGAAAAAAG
AGAACATCACCTTGTAAAACTGAAGATTGTGACCAGTCAGAATAATGTCA
AAGTGCTTACAGTGCAGGTAGTGATATGTGCATCTACTGCAGTGAAGGCA
CTTGTAGCATTATGGTGACAGCTGCCTCGGGAAGCCAAGTTGGGCTTTAA
AGTGCAGGGCCTGCTGATGTTGAGTGCTTTTTGTTCTAAGGTGCATCTAG
TGCAGATAGTGAAGTAGATTAGCATCTACTGCCCTAAGTGCTCCTTCTGG
CATAAGAAGTTATGTATTCATCCAATAATTCAAGCCAAGCAAGTATATAG
GTGTTTTAATAGTTTTTGTTTGCAGTCCTCTGTTAGTTTTGCATAGTTGC
ACTACAAGAAGAATGTAGTTGTGCAAATCTATGCAAAACTGATGGTGGCC
TGCTATTTCCTTCAAATGAATGATTTTTACTAATTTTGTGTACTTTTATT
GTGTCGATGTAGAATCTGCCTGGTCTATCTGATGTGACAGCTTCTGTAGC
ACTAAAGTGCTTATAGTGCAGGTAGTGTTTAGTTATCTACTGCATTATGA
GCACTTAAAGTACTGCTAGCTGTAGAACTCCAGCTTCGGCCTGTCGCCCA
ATCAAACTGTCCTGTTACTGAACACTGTTCTATGGTTAGTTTTGCAGGTT
TGCATCCAGCTGTGTGATATTCTGCTGTGCAAATCCATGCAAAACTGACT
GTGGTAGTGAAAAGTCTGTAGAAAAGTAAGGGAAACTCAAACCCCTTTCT
ACACAGGTTGGGATCGGTTGCAATGCTGTGTTTCTGTATGGTATTGCACT
TGTCCCGGCCTGTTGAGTTTGGTGGGGATTGTGACCAGAAGATTTTGAAA
ATTAAATATTACTGAAGATTTCGACTTCCACTGTTAAATGTACAAGATAC
ATGAAATATTAAAGAAAATGTGTAACTTTTTGTGTAAATACATCTTGTCT
TGTTTTCATTCAAAAACATTTCACTTTTGGGGTTGCGTGTCAGATTTGGC
AGTATAAATTCTGGCTATATTTTTTGTTGTTAGATTTATTTGGCTGTTAA
GTATTGCGATATGACTAAACATACTGTATACCTGATGATCATCTGTAAAG
TTAGAGTATATCTTTTTGCTTTCTTTGGAGTTAGTGTTATTCCAGGATAT
TTTACTTAATCTAAAAGTTAATTTATGTTGCTCATATATTACTCAAGTAT
TTAAATTTAGAGAGAATGCCGCTCTGTTTAAAGCAATGTGTAAAGATGAG
TTTTTTTAAGCATGGAATTTAGGGTTGGGGTACAATTTGTTTCTATTAAG
CAAGTACCAGTTTACCAATACATGAGTAACTGAAGTGTAACTGTTAAATG
CTTGTATACTAGTTTTTCTTTCTGATTGTCAGTGATTTATAAGCTATAAA
TGACCAAGGTCCTCAGACTGCTTTTAGCATCTGCAACTTAAAAAAATGGG
AGTTAGAAAAAGAACAAATGCTAAATAGAGTAACAGTTAAATGTATGTGT
ACACTCTTCCCAAATGCCAAGAGTGCAGCGGTGGGGTGAGATTCAGATAT
TCATTTATTTCTAAGTCTGTAGTTAACATTTATGTTCCCTACTCCCTACG
TAAGCCAGACTTTGGCAACAGTGATAGTTGATTCCAGGCTTATTTGACTT
AAAGTCACTGAAGTGGAAACTAAGAAGTGGCAGTTAGTGTTTTACCCAGC
ATTTCTGCCTTCTCTCTTTTCTTCATGTGTTTTTGTCTCTAGCCTATGTG
TATTTGTGTAGAATAATGTGGGATACCTGAATAATAGATTTAAAAGGACC
AAGTGGTAAAATTGGGCCCAAGCTGAAGTACAGGCAAACTTGATGTTTGA
AAGATAAGTTTTGAGAAATGTCATTGTATTTTGGAGTAAAAGAGGCTATC
TTAGTAATAAGAAATAAACTTCCATAACACTAGGTTAGACCACCCAATAA
ATCTAGAAATCAGCTTTTAAAAATATTGTCTGAAGTCTAACAAAAGTTTT
CACCTCTAATGTGTTCTTTAAGAAATTTAAGGAACTTAGCCTTGGATTCC
TGAATAGAAAGGTAAGAATTCTATCATTCTGGAGTTGATGAAAACATAAA
TTTTCAGGATGTGAAATGAACAGTGATTTATAAAATGGAAATCAAATTGT
ACATTAGCAGAGTTCTTAAGCTTTTTGAATTGAAGGAGACCTAATAATTG
TGTCTTTTTGGTTATTTAGTGACAAACGTGGCTTTCAAACTATGCTTAAA
AAGTTCCGGCTGGACACGGTGGCTCACACCTATAATCCTAGCACTTGGGG
AGGCTGAGGCAGACGGATTACCTGAGGTCAGGAGTTCGAGACCAACCTGG
CCGACATGGTGAAACGCTGTCTCTACTAAAAATATAAAAAATTAGCCGGG
TGCAGTGGCGTGCACCTGTAATCCCAGCTACTCTGGAGGCTGAGGCAGGA
GAATCACCTGAACCTGGGAGGTGGAGGTTTCAGTGAGCTGAGATCCTGCC
ACTGCACTCCAGCCTGGGCGCAAGACCAAGACTTAAACGCAAAAAAAAAA
AAAAAAAAAAAAAAAAAGTTTCATAATACAGCATGGTCTGGTAGTTTGCA
AAATGGTGTGCTTTTGGGGAGATACACTAGCAATTTTTTTAAAAACTGGA
ACAGTGTGATAGGAAGCCTGCTGGATGATTTCTTAAATATTCTAAAATGT
AAGTCAAATATGTTTTAATAACAAAGACTTAAATGGCTTTTCTCCCTAGA
GACTGAAACTAGTATTCATTGTGTTCAGAACTTAATTGGGCTTGAACTGA
GATTTAAATCTAATAAACAAGTTAATAAATGTGTATGTTTTGTTGTGGGT
TTGGTAGTGATCTGTGGTTCTATAGGGTTTAATAGGAATTGCTTTTGATT
TGTTTCTGGCTTTAGAATGTGAGGCAAATTTTACATTCTTGGTTCTATTA
AGATTTTCTTAGGCATGCTAACATGCCAACAAAAAGCCATGTAAGTATTG
TATAAAAAGATTCACATTGTTAATTTAGCCATTTTGAAATTCAGATGAGT
GAGCAAGTTGATAATGGCCTCATCTCTGACCTGAGAAAAAACAACTTTGA
CCCTTGTTCTTAAAATGCTTTAACCTTGAAGTTGCTTGAGACTTAAGAGG
TCATGTTGCTTTAGGTTTAATAAATAGCCTTAACTATTTGGAGGGGAAAA
AATGGGTCAACTTTTTTTTTTTTTTTTGGCGTTTGCATGTACAACTTTCT
ATTTTTAGCCTATATTTGGAAAGAAAGCACTTAACATTTTAGGAATTCTT
TTTAAAGCTGCTTGCAAAGTGTTGGTGATTTTACTGAAAACTTTTGAGAT
CTTCATTTTACAGGCAGACCTGTCTAACTACAAGCCAGACTTGGGTTTTC
TCCTGTAGTTTGAAGACACACTGACTCCTGACAAAATGCAGCCTGCAACT
TCCTGGAGAACAACTCAGTGTCACATTAAAGTTTATTATGTATTTAATGA
TACACTGTTTAATTGACAGTTTTGCATAGTTTGTCTAACTTTAGAGAATT
AAGAGCCTCTCAACTGAGCAGTAAAGGTAAGGAGAGCTCAATCTGCACAG
AGCCAGTTTTTAGTGTTTGATGGAAATAAGATCATCATGCCCACTTGAGA
CTTCAGATTATTCTTTAGCTTAGTGGTTGTATGAGTTACATCTTATTAAA
GTCGAAATTAATGTAGTTTTCTGCCTTGATAACATTTCATATGTGGTATT
AGTTTTAAAGGGTCATTAGGAAAATGCACATATTCCATGAATTTTAAGAC
CCATAGAAAAGTTGAAGAATGCTTAATTTTCTTATCCAGTAATGTAAACA
CAGAGACAGAACATTGAGATGTGCCTAGTTCTGTATTTACAGTTTGGTCT
GGCTGTTTGAGTTCTAGCGCATTTAATGTTAATAAATAAAATACTGCATT
TTAAAGCTGTTAAGAAATTGTCCAGAACGAGAATATTGAAATAAAAACTT
CAAGGT
Pre-mir-17
CAAAGUGCUUACAGUGCAGGUAGUGAUAUGUGCAUCUACUGCAGUGAAGG
CACUUGUAG
Pre-mir-18a
UAAGGUGCAUCUAGUGCAGAUAGUGAAGUAGAUUAGCAUCUACUGCCCUA
AGUGCUCCUUCUGG
Pre-mir-19a
AGUUUUGCAUAGUUGCACUACAAGAAGAAUGUAGUUGUGCAAAUCUAUGC
AAAACUGA
Pre-mir-19b
AGUUUUGCAGGUUUGCAUCCAGCUGUGUGAUAUUCUGCUGUGCAAAUCCA
UGCAAAACUGA
Pre-mir-20a
UAAAGUGCUUAUAGUGCAGGUAGUGUUUAGUUAUCUACUGCAUUAUGAGC
ACUUAAAG
Pre-mir-92a-1
AGGUUGGGAUCGGUUGCAAUGCUGUGUUUCUGUAUGGUAUUGCACUUGUC
CCGGCCUGU

The inhibitor of CPSF3, ISY1, or SF3B1 may be any inhibitor of CPSF3, ISY1, or SF3B1 known in the art or described herein. The inhibitor may inhibit the level and/or activity of CPSF3, ISY1, or SF3B1. Levels of CPSF3, ISY1, or SF3B1 (e.g., mRNA level or protein level) can be measured using a method known in the art or described herein, such as by Northern blot analysis, q.RT-PCR, sequencing technology, RNA in situ hybridization, in situ RT-PCR, oligonucleotide microarray, immunoassays (e.g., Western blot, immunohistochemistry and ELISA assays), Mass spectrometry, or multiplex bead-based assays. The activity of CPSF3, ISY1, or SF3B1 may also be measured using a method known in the art or described herein, e.g., by measuring a level of pro-miRNA or a level one or more of miR-17, miR-18a, miR-19a, miR-20a, or miR-19b.

In some embodiments, the inhibitor is a small molecule, an antisense oligonucleotide, a small interfering RNA (siRNA), a microRNA (miRNA), or an antibody. Methods of making such inhibitors are known in the art. The antibody may be a full-length antibody or an antigen-binding fragment thereof, such as a Fab, F(ab)2, Fv, single chain antibody, Fab or sFab fragment, F(ab′)2, Fd fragments, scFv, or dAb fragments. Methods for producing antibodies and antigen-binding fragments thereof are well known in the art (see, e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). The small molecule may be, in some embodiments, an organic compound having a molecular weight of below 900, below 800, below 700, below 600, or below 500 daltons. Methods of making such small molecules are known in the art. Antisense oligonucleotides may be modified or unmodified single-stranded DNA molecules of less than 50 nucleotides in length (e.g., 13-25 nucleotides in length). siRNAs may be double-stranded RNA molecules of about 19-25 base pairs in length with optional 3′ dinucleotide overhangs on each strand. Antisense oligonucleotides and siRNAs are generally made by chemical synthesis methods that are known in the art. MicroRNAs (miRNAs) are small non-coding RNA molecules. They may be transcribed and then processed from a primary-microRNA (pri-miRNA) to a progenitor-microRNA (pro-miRNA) to a pre-microRNA (pre-miRNA), and finally to a mature miRNA, which can act as an inhibitor. miRNAs may be produced in a subject by delivering a gene that encodes the pri-miRNA, which is then processed in the subject to a mature miRNA.

In some embodiments, the inhibitor of SF3B1 is selected from the group consisting of FR901463 (Fujisawa Pharmaceutical Co.), FR901464 (Fujisawa Pharmaceutical Co.), FR901465 (Fujisawa Pharmaceutical Co.), spliceostatin A (SSA, Sigma), a sudemycin, a meayamycin, a pladienolide (e.g., pladienolide A-G or E7107, Eisai Inc.) and GEX1 (Kyowa Hakko Kogyo Co., Ltd.). Such inhibitors are known in the art or commercially available (see, e.g., Bonnal et al. (2012) Nature Reviews: Drug Discovery. Vol 11:847-859, Fan et al. (2011) ACS Chem Biol. Vol 6(6):582-589).

An effective amount is an agent or inhibitor as described herein is an amount that is sufficient to provide a medically desirable result, such as treatment of cancer or inhibition of formation of a progenitor-microRNA. The effective amount will vary with the particular disease or disorder being treated, the age and physical condition of the subject being treated, the severity of the condition, the duration of the treatment, the nature of any concurrent therapy, the specific route of administration and the like factors within the knowledge and expertise of the health practitioner. For administration to a subject such as a human, a dosage of from about 0.001, 0.01, 0.1, or 1 mg/kg up to 50, 100, 150, or 500 mg/kg or more can typically be employed.

An agent or inhibitor as described herein and compositions thereof can be formulated for a variety of modes of administration, including systemic, topical or localized administration. A variety of administration routes are available. The particular mode selected will depend upon the type of cancer or other disease being treated and the dosage required for therapeutic efficacy. The methods of the disclosure, generally speaking, may be practiced using any mode of administration that is medically acceptable, meaning any mode that produces effective levels of the active compounds without causing clinically unacceptable adverse effects. Such modes of administration include, but are not limited to, oral, rectal, topical, nasal, intradermal, or parenteral routes. The term “parenteral” includes subcutaneous, intravenous, intramuscular, or infusion. The pharmaceutical compositions described herein are also suitably administered by intratumoral, peritumoral, intralesional or perilesional routes, to exert local as well as systemic effects.

Techniques and formulations generally can be found in Remington: The Science and Practice of Pharmacy, Pharmaceutical Press; 22nd edition and other similar references. When administered, an agent or inhibitor as described herein may be applied in pharmaceutically-acceptable amounts and in pharmaceutically-acceptable compositions. Pharmaceutical compositions and pharmaceutically-acceptable carriers are also described herein. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the disclosure. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

### Compositions and Pharmaceutically-Acceptable Carriers

Other aspects of the disclosure relate to compositions comprising an agent or inhibitor as described herein, e.g., for use in treatment of cancer. In some embodiments, the composition is a pharmaceutical composition. In some embodiments, the composition comprises an agent or inhibitor as described herein and a pharmaceutically-acceptable carrier. In some embodiments, the composition is for use in treating cancer. In some embodiments, the composition is for use in modulating progenitor-microRNA (pro-miRNA) levels.

The term “pharmaceutically-acceptable carrier” as used herein means one or more compatible solid or liquid filler, diluents or encapsulating substances which are suitable for administration into a subject, e.g., a human. A pharmaceutically acceptable carrier is “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the composition.

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the pharmaceutical composition may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For topical administration, the pharmaceutical composition can be formulated into ointments, salves, gels, or creams, as is generally known in the art. Topical administration can utilize transdermal delivery systems well known in the art. An example is a dermal patch.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the agent or inhibitor, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments, the pharmaceutical compositions used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The agent or inhibitor described herein and/or the pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

### Method of Modulating Progenitor-microRNA (Pro-miRNA) Levels

Other aspects of the disclosure relate to a method of modulating (e.g., reducing) progenitor-microRNA (pro-miRNA) levels in a cell. In some embodiments, the method comprises contacting the cell with an agent that inhibits formation of a progenitor-microRNA (pro-miRNA). In some embodiments, contacting the cell with the agent reduces the levels of one or more of miR-17, miR-18a, miR-19a, miR-20a, or miR-19b in the cell. As used herein, “a reduced level of one or more of miR-17, miR-18a, miR-19a, miR-20a, or miR-19b” means that the level of one or more of miR-17, miR-18a, miR-19a, miR-20a, or miR-19b is below a control level, such as a pre-determined threshold or a level of one or more of miR-17, miR-18a, miR-19a, miR-20a, or miR-19b in a control sample (e.g., a cell that has not been contacted with the agent). A reduced level of one or more of miR-17, miR-18a, miR-19a, miR-20a, or miR-19b includes a level that is, for example, 1%, 5%, 10%, 20%, 30%, 40%, 50%, 60%, 70%, 80%, 90%, 100%, 150%, 200%, 300%, 400%, 500% or more below a control level.

In some embodiments, the agent is an inhibitor of CPSF3, ISY1, or SF3B1. In some embodiments, the inhibitor is a small molecule, an antisense oligonucleotide, a small interfering RNA (siRNA), a microRNA (miRNA), or an antibody. Such inhibitors are described herein.

The cell may be any cell. In some embodiments, the cell is a cancer cell. In some embodiments, the cell is in a subject (e.g., a cancer cell in a subject, such as a human subject). In some embodiments, the cell is ex vivo (e.g., in cell culture).

### Methods of Screening

Other aspects of the disclosure relate to a method of screening for an inhibitor of microRNA (miRNA) biogenesis. In some embodiments, the method comprises contacting a cell expressing a primary microRNA 17˜92 (pri-miR-17˜92) with a candidate substance; measuring a ratio of the level of miR-17, miR-18a, miR-19a, miR-20a, and/or miR-19b to the level of miR-92; and identifying the candidate substance as an inhibitor of miRNA biogenesis if the ratio is decreased compared to a control ratio.

The measuring may be accomplished using any method known in the art or described herein. In some embodiments, the measuring comprises a luciferase assay, such as the assay described in Example 1. In some embodiments, the luciferase assay comprises use of a Renilla Luciferase gene, wherein a 3′UTR of the Renilla Luciferase gene contains a primary microRNA-17˜92 (pri-miR-17˜92), or a fragment thereof.

In some embodiments, the control ratio is the ratio in a cell that has not been contacted with the candidate substance.

In some embodiments, the candidate substance is a small molecule. In some embodiments, the candidate substance is a member of library (e.g., a library of small molecules). The library may contain, e.g., at least 20, 50, 100, 200, 500, 1000, 10,000, 100,000, 1,000,000 or more members. Some or all members of a library may be screened using a method provided herein, e.g., by high-throughput screening using assay plates or drop-based microfluidics.

Variant primary microRNAs (pri-miRNAs)

Other aspects of the disclosure relate to a variant primary microRNA (pri-miRNA), e.g., that is incapable of forming a progenitor-microRNA (pro-miRNA). In some embodiments, the variant pri-miRNA is not processed or not capable of being processed by CPSF3. In some embodiments, the variant pri-miRNA comprises a mutation in a CPSF3 cleavage domain. A CPSF3 cleavage domain is an RNA sequence that CPSF3 is capable of cleaving. An RNA sequence can be determined to be a CPSF3 cleavage domain, e.g., by contacting the RNA with CPSF3 in vitro and measuring the level of full-length and cleaved RNA produced after the contacting. In some embodiments, the variant pri-miRNA is a variant pri-miR-17˜92.

In some embodiments, the variant pri-miRNA comprises a mutation (e.g., a deletion or substitution mutation) in the sequence CAGUCAGAAUAAUGU. In some embodiments, the mutation is a mutation (e.g., a deletion or substitution mutation) in the second A and/or the second C in the sequence CAGUCAGAAUAAUGU. In some embodiments, the mutation is a substitution mutation (e.g., replacement of an A with C, G, or U and/or replacement of a C with A, G, or U).

In some embodiments, a vector is provided, comprising a coding sequence encoding a variant pri-miR as described herein. The vector may be a plasmid or viral vector (e.g., a lentiviral, retroviral, adenoviral, or adeno-associated viral vector).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### A Biogenesis Step Upstream of Microprocessor Controls miR-17˜92 Expression

### Introduction

MicroRNAs (miRNAs) represent a large family of regulatory RNAs that inhibit target gene expression by base pairing with complementary sites in the 3′ untranslated region (3′UTR) to promote messenger RNA (mRNA) decay and translational repression (Bartel, 2009). The current model of canonical miRNA biogenesis involves the two-step processing of long primary miRNA transcripts (pri-miRNAs) by the Microprocessor, comprising the ribonuclease DROSHA and its essential co-factor, the double-stranded RNA-binding protein DGCR8, to generate 50-70 nucleotide (nt) precursor miRNA (pre-miRNA) intermediates that are processed by the double-stranded ribonuclease DICER to mature ˜22 nucleotide miRNAs (Denli et al., 2004; Gregory et al., 2004; Ha and Kim, 2014). Individual pri-miRNA can be expressed from distinct miRNA loci, or from the introns or exons of protein coding genes. Furthermore some pri-miRNAs contain a single miRNA whereas other miRNAs are processed from pri-miRNAs containing clusters of several miRNAs. Regardless, Microprocessor recognizes the hairpin structures in the pri-miRNA through the stem-loop and the stem-loop-ssRNA junction and specifically cleaves both the 5′ and 3′ flanking segments to generate pre-miRNA (Ha and Kim, 2014). Pre-miRNAs are exported to the cell cytoplasm by Exportin-5 (XPO5) where they are further cleaved by a complex comprising the ribonuclease DICER and the double-stranded RNA-binding protein TRBP2, generating mature miRNA duplexes (Ha and Kim, 2014). The 5′ or 3′ miRNA is selected and loaded into the RNA-induced silencing complex (RISC) that recognizes sites in the 3′ untranslated region (UTR) of target mRNAs to repress protein expression (Bartel, 2009).

miRNAs play critical roles in normal development and their dysregulation can cause disease (Di Leva and Croce, 2010; Mendell and Olson, 2012). miRNA expression can be regulated at the level of pri-miRNA transcription but it is increasingly well appreciated that posttranscriptional mechanisms play an important role controlling miRNA expression (Siomi and Siomi, 2010). Several Microprocessor- or Dicer accessory factors, and inhibitory proteins have been identified that either facilitate or inhibit distinct subsets of miRNAs. Moreover the activity of some of these factors is linked with cell-signaling pathways to afford dynamic control of the miRNA biogenesis machinery (Mori et al., 2014; Siomi and Siomi, 2010). Perturbation of these pathways can be oncogenic. One well-characterized example of the posttranscriptional control of miRNA expression involves the RNA-binding protein LIN28 that selectively represses let-7 biogenesis embryonic stem cells (ESCs) and during early embryonic development (Heo et al., 2008; Nam et al., 2011; Newman et al., 2008; Rybak et al., 2008; Viswanathan et al., 2008). LIN28 recruits the terminal uridylyl transferase (TUTase) ZCCHC6 and/or ZCCHC11 to promote pre-let-7 decay by DIS3L2 (Chang et al., 2013; Faehnle et al., 2014; Hagan et al., 2009; Heo et al., 2009; Thornton et al., 2012; Ustianenko et al., 2013). This pathway helps maintain an undifferentiated cell state and is often reactivated in cancer (Viswanathan et al., 2009).

To investigate how expression of other miRNAs might be posttranscriptionally regulated, the polycistronic miR-17˜92 cluster was studied. Pri-miR-17˜92 encodes six (miR-17, -18a, -19a, 20a, -19b-1, and -92a) mature miRNAs. Haploinsufficiency of this locus causes the Feingold syndrome of microcephaly, short stature, and digital abnormalities in human patients and mouse models, whereas ablation of this locus in mouse causes perinatal lethality with heart, lung, and B cell defects, thereby highlighting the importance of precise control of miRNA expression from this cluster (Concepcion et al., 2012; de Pontual et al., 2011; Mendell, 2008; Ventura et al., 2008). Conditional mouse knockout approaches underscore the importance of this miRNA cluster for kidney development and function, and neural stem cell biology (Bian et al., 2013; Marrone et al., 2014; Patel et al., 2013). Strikingly, gene amplification and increased expression of miRNAs from this cluster is observed in numerous types of cancer compared to normal tissues, and transgenic overexpression of this ‘OncomiR-1’ promotes B-cell lymphoma, T-cell acute lymphoblastic leukemia (T-ALL), and retinoblastoma in mice (Conkrite et al., 2011; He et al., 2005; Mavrakis et al., 2010; Nittner et al., 2012; Sandhu et al., 2013). Individual miRNAs within this cluster are known to promote cell proliferation, inhibit apoptosis, inhibit differentiation, and promote angiogenesis, as well as other hallmarks of cancer to drive tumorigenesis (Mendell, 2008; Mu et al., 2009; Olive et al., 2009). Moreover, while expression of miR-19 promotes lymphoma in mouse, co-expression of miR-92 suppresses this oncogenic activity(Olive et al., 2013). The miR-19:miR-92 expression ratio in Myc-induced mouse tumors appears to be dynamically regulated during lymphoma progression (Olive et al., 2013). Similarly, whereas ectopic expression of the entire miR-17˜92 cluster can result in the expansion of apparently normal multipotent hematopoietic progenitors, the imbalanced expression of miR-19 or miR-92 results in B-cell hyperplasia and erythroleukemia, respectively (Li et al., 2012). Co-expression of miR-17 suppressed the miR-92 oncogenic effects in this context. Consistent with these mouse models, elevated miR-92 and decreased miR-17 expression was observed in B-cell chronic lymphocytic leukemia patients with an aggressive clinical phenotype (Li et al., 2012). Taken together the precise regulation of this miRNA cluster, and importantly, the relative expression of individual miRNAs from within this cluster are critical for development and disease yet the mechanisms that control miR-17˜92 biogenesis remain largely unknown (Guil and Caceres, 2007; O'Donnell et al., 2005).

The expression of individual miRNAs from pri-miR-17˜92 is found to be dynamically regulated during ESC differentiation. A new paradigm for miRNA regulation in which certain sequences (repression domains) within the pri-miR-17˜92 are involved in the formation of a higher-order RNA conformation that selectively inhibits Microprocessor-mediated production of pre-miR-17, -18a, -19a, 20a, and -19b, from this cluster is described. Cleavage of pri-miR-17˜92 to remove the autoinhibitory 5′ fragment produces a new miRNA biogenesis intermediate that has been termed ‘progenitor-miRNA’ (pro-miRNA). Pro-miRNA biogenesis is dynamically regulated and specifically requires the endonuclease component of the Cleavage and Polyadenylation Specificity Factor complex, CPSF3 (also known as CPSF73 or CPSF-73) (Mandel et al., 2006), as well as the poorly characterized spliceosome factor ISY1. These factors are selectively required for the expression of all miRNAs within the cluster except for miR-92. Thus, developmentally regulated generation of pro-miRNA explains the posttranscriptional control of miR-17˜92 expression. The findings challenge the current two-step processing model for miRNA biogenesis and add an additional processing step upstream of Microprocessor that can be dynamically regulated for precise miRNA control.

### Experimental Procedures

Cell Culture, ESC Differentiation, and Cell Transfections. Mouse ESCs (V6.5, Dgcr8−/−, Dicer−/−, and miR-17˜92−/−) were cultured in DMEM with ESGRO (1000 units/mL), supplemented with 15% (v/v) FBS and antibiotics. Flag-DROSHA-293, and HEK293 cells were cultured in DMEM with 15%(v/v) FBS (Gregory et al., 2004). For ESC differentiation, ESGRO was removed from the media, and cells collected daily. Lipofectamine 2000 (Invitrogen) was used for both DNA and siRNA transfections according to the manufacturer's instructions.

Plasmids and Site-Directed Mutagenesis. The cDNA of mouse pri-miR-17˜92 was generated by PCR, and cloned into EcoRI and XhoI sites of pcDNA3 (Invitrogen), as well as the XhoI and NotI sites of psiCHECK™-2 (Promega). The cDNA of mouse ISY1 and CPSF3 were PCR amplified and cloned into the BamHI and SalI sites of pFlag-CMV2 (Sigma) and the cDNA of CPSF3 was also cloned into the SalI and NotI sites of pETDuet-1 Vector (Novagen). pFlag-CMV2-DGCR8 plasmid was as described before (Gregory et al., 2004). Primers used for CRISPR/Cas9 mutagenesis were designed on line (crispr.mit.edu/) and cloned into PX330 vector. Q5® Site-Directed Mutagenesis Kit (NEB) was used for both mutagenesis and for repression domain deletion following the manufacturer's instructions. All the primers used for plasmid construction are listed in Table 2.

TABLE 2
List of Primers Used for Cloning

| Name | Sequence (5′→3′) |
|---|---|
| Mouse ISY1_CDS_SalI_F | GGTCGACATGGCCCGAAATGCAGAA |
| Mouse ISY1_CDS_BamHI_R | CGGATCCCTAGTACCCCAGGAGTCGCTT |
| Mouse CPSF3_CDS_SalI_F | GGTCGACATGTCTGCGATTCCTGCTGAGGA |
| Mouse CPSF3_CDS_BamHI_R | CGGATCCTCAGTGAACTGGCGTCAGGGCC |
| Mouse CPSF3_CDS_Not1_R | GCGGCCGCTCAGTGAACTGGCGTCAGGGCC |
| Mouse pri_miR-17~92_EcoR1_F | CGGAATTCCCTAGTCATACACGTGGACCTA |
| Mouse pro-pri_miR-17~92_EcoR1_F | CGGAATTCGTCAGAATAATGTCAAAGTGCT |
| Mouse pri_miR-17~92_Xho1_R | CGCTCGAGTCAGTGCTACAAAATAACTTT |
| Mouse pro-pri_miR-17~92_Xho1_R | CGCTCGAGCCAAACTCAACAGGCCGGGACAA |
| Mouse pri_miR-17~92_Xho1_F | CGCTCGAGCCTAGTCATACACGTGGACCTA |
| Mouse pre_miR-17_3P_Not1_R | TGCGGCCGCCAGTAGATGCACACATCACT |
| Muta_miR-17~92_AG_CC_F | GTGACCAGTCccAATAATGTCAAAGTG |
| Muta_miR-17~92_AG_CC_R | AGCTTCAGTCCCAAGGTG |
| Muta_miR-17~92_G_C_F | GTGACCAGTCAcAATAATGTCAAAG |
| Muta_miR-17~92_G_C_R | CAGCTTCAGTCCCAAGGTG |
| Muta_miR-17~92_A_C_F | GTGACCAGTCcGAATAATGTCAAAG |
| Muta_miR-17~92_A_C_R | AGCTTCAGTCCCAAGGTG |
| Muta_miR-17~92_C_A_F | TGTGACCAGTaAGAATAATGTCAAAG |
| Muta_miR-17~92_C_A_R | GCTTCAGTCCCAAGGTGAG |
| Muta_miR-17~92_CA_GG_F | TGTGACCAGTggGAATAATGTCAAAGTGC |
| Muta_miR-17~92_CA_GG_R | GCTTCAGTCCCAAGGTGAG |
| Del_miR-17~92_RD_F | AATTATTTATTTCAAATTTAGCAGGAATAAAG |
| Del_miR-17~92_RD_R | TCTGTCACAAACATCAATTAC |
| Del_miR-17~92_RD*_F | CAGGTTGGGATTTGTCGC |
| Del_miR-17~92_RD*_R | CCCTTACTTCTCTACAGAC |
| Sub_miR-17~92_RD_F | aagagaAATTATTTATTTCAAATTTAGCAGGAATAAAG |
| Sub_miR-17~92_RD_R | cattccAAAAAGCCAAAGCTCTAAATTC |
| Sub_miR-17~92_RD*_F | cattccGAAAATCAAACCCCTTTCTAC |
| Sub_miR-17~92_RD*_R | aagagaACAGACTTTTCACCACCAC |
| Muta_CPSF3_75DK_76HA_F | TTTCCATTTGaaggcCTGTGGAGCCCTGC |
| Muta_CPSF3_75DK_76HA_R | TGACTGATCAACAGAAGG |
| Mouse_pri_miR-17~92_EcoR1_40F | CGGAATTCTAAAGTGAACCTCACCTTGGGA |
| Mouse_pri_miR-17~92_EcoR1_80F | CGGAATTCTTTGTCTAATTATTTATTTCAAAT |
| Mouse_pri_miR-17~92_EcoR1_120F | CGGAATTCTTTGTGACAGAATTTAGAGCTT |
| Mouse_pri_miR-17~92_EcoR1_160F | CGGAATTCCTTCTGGCTATTGGCTCCTCCC |
| Mouse_pri_miR-17~92_EcoR1_200F | CGGAATTCAAGGCTTACATGTGTCCAATT |
| Mouse_pri_miR-17~92_EcoR1_300F | CGGAATTCTTTGTAGGACCCTTATTGTGT |
| Mouse_pri_miR-17~92_EcoR1_400F | CGGAATTCTATCTGACAATGTGGAGGAC |
| Mouse_pri_miR-17~92_EcoR1_500F | CGGAATTCTATTTCTAATTTGGGGTGATA |
| Mouse_pri_miR-17~92_EcoR1_600F | CGGAATTCAATAAATACAGTTTGAACTCTTG |
| Mouse_pri_miR-17~92_EcoR1_700F | CGGAATTCGTGAGTATATTCTAGTTTTGTAGCTA |
| CRISP_pre_miR17_F | CACCGAATAAAGTGAACCTCACCTT |
| CRISP_pre_miR17_R | AAACAAGGTGAGGTTCACTTTATTC |
| CRISP_pre_miR17_AG_CC | ACAGAATTTAGAGCTTTGGCTTTTTCCTTTTT |
| | GTCTAATTATTTATTTCAAATTTAGCAGGAATA |
| | AAGTGAACCTCACCTTGGGACTGAAGCTGTGAC |
| | CAGTCCCAATAATGTCAAAGTGCTTACAGTGCA |
| | GGTAGTGATGTGTGCATCTACTGCAGTGAGGGC |
| | ACTTGTAGCATTATGCTGACAGCTG |

RNA Purification and Detection of Large and Small RNAs by Northern Blot. Total RNA was extracted from each sample using Trizol reagent (Invitrogen). 200 micrograms (μg) total RNA was used for polyA(+) RNA isolation through the Dynabeads® mRNA Purification Kit (Invitrogen) following the manufacturer's instructions, while the supernatant in the step of the binding of oligo(dT) cellulose was kept and an equal volume of Isopropanol added to precipitate PolyA(−) RNA. 200 ng polyA(+), 20 μg polyA(−) and 20 μg total RNA were loaded on 15% Formaldehyde-Agarose gels for large RNA Northern blot. The cDNAs amplified by PCR corresponding to the different regions of mouse pri-miR-17˜92 were labeled by ³²P-dCTP using DNA Polymerase I, Large (Klenow) Fragment (NEB) and used as probes. Small RNA Northern blot was performed as previously described (Gregory et al., 2004) using 15 μg of total RNA. Probes and primers used for amplifying the probes were all listed in Table 3.

TABLE 3
List of Probes Used for
Northern Blot and 5′ RACE Primers

| Name | Sequence (5′→3′) |
|---|---|
| miR-17/20_R | CTACCTGCACTATAAGCACTTTA |
| miR-18a_R | CTATCTGCACTAGATGCACCTTA |
| miR-19_R | TCAGTTTTGCATAGATTTGCACA |
| miR-92_R | CAGGCCGGGACAAGTGCAATA |
| miR-17~92_P1_F | GCTCGTGGTTCTTAGGTGA |
| miR-17~92_P1_R | GCAGCAAGCCTGAACTCTA |
| miR-17~92_P2_F | GGTGAGTATATTCTAGTTTTGTA |
| miR-17~92_P2_R | TCGACAAGATTAATGATTATTGTT |
| miR-17~92_P3_F | CTCATCCAATCCAAGTCAAGCAA |
| miR-17~92_P3_R | ACACCACGCAGGCTCTACATCGA |
| miR-17~92_P4_F | TGGTTTGAAGAGTCAGTCCTAC |
| miR-17~92_P4_R | AGCACGCACTTCCTGAATA |
| RP2_1 | AGTGCTACAGAAGCTGTCACATCACAC |
| RP2_2 | CTGGAGTTCTACAGCTGGCAGTACTTT |
| RP2_3 | ACACCACGCAGGCTCTACATCGA |
| RP1_1 | TGGCAGGCTTCCTATGTT |
| RP1_2 | GCAGGCTTCCTATGTTGGA |
| RP1_3 | CTTCTTACTAAATCCAGCGAGC |
| Pre-miR-92_R | GTGTAGAAAGGGGTTTGATTTTCC |
| Pre-miR-19b_R | AACCATAGACCAGTGCTCAATAAC |

mRNA-seq, Small RNA-seq, and Bioinformatics Analysis. 200 ng polyA(+) RNA isolated as described above was used for mRNA-seq. Sample preparation was with the TruSeq Stranded mRNA Sample Prep Kits (Illumina). Small RNA-seq sample preparation was performed as previously described (Thornton et al., 2014). Both sets of samples were subjected to Illumina high-throughput sequencing. For the analysis of mRNA-seq data, Top-hat software was used. Bowtie software was used for the alignment of small RNAs to mature miRNA sequences (www.mirbase.org/) without any mismatches permitted.

5′ RACE. 50 ng polyA(+) RNA and 5 μg PolyA(−) RNA were used for 5′ RACE through the 5′ RACE System (Invitrogen) following the manufacturer's instructions. Gene specific primers were used for reverse transcription, and then cDNAs were purified and a dC-tailadded using TDT. Two rounds of PCR were performed to amplify the PCR product, which were cloned into pGEM-T Easy vector (Promega). Different clones were picked for Sanger sequencing. Primers used for 5′ RACE were listed in Table 3.

In vitro Transcription, Microprocessor, and CPSF3 Cleavage Assays. A T7 primer and gene specific primers were used to PCR amplify pri-miR-17˜92 sequences from plasmid DNA templates. PCR products were gel-purified and used as templates for in vitro Transcription using the Riboprobe® (Promega) system together with ³²P-CTP for radioactive labeling. Microprocessor purified from Flag-DROSHA-293 cells was used for Microprocessor Assays as previously described (Gregory et al., 2004). Cold RNA was produced using the same strategy without using ³²P-CTP. For RNA annealing, 10 mM MgCl₂ was added to 200 pmol cold RNA and incubated at 95° C. for 5 min, and then slowly cooled to RT. Annealed RNA was subjected to 5% native Polyacrylamide Gel for Ethidium bromide staining and used for Microprocessor assay followed by small RNA Northern blot analysis. His-CPSF3 complex was purified from E. coli as described previously for other proteins (Chang et al., 2013; Piskounova et al., 2011). Assays were performed using the same condition as for Microprocessor Assays described above. For RNA substrate, portions of pri-miR-17˜92 were in vitro transcribed and used as a substrate.

CRISPR/Cas9 Mutagenesis. 0.5 pmol 200 nt oligo DNA corresponding to mouse pri-miR-17˜92 sequence containing AG→CC mutation, 12 μg PX330 plasmid containing guide RNA sequence near the cleavage site and 1 μg plasmid expressing puromycin resistance gene were co-transfected into 3 million V6.5 ESCS by nucleofection using Primary Cell Nucleofector™ Kits (Lonza). After one day, puromycin was added to media to select positive ES cell clones for two days. Finally individual ESC clones were picked and screened by PCR and DNA sequencing.

Synthetic RNA Annealing. Synthetic RD and RD* RNAs were used for the annealing assay. 50 μm each RNAs were dissolved in 1× annealing buffer (10 mM Tris, pH 8.0, 20 mM NaCl). The solution was incubated for 1 min at 95° C. and cooled slowly to room temperature. Annealed RNA was subjected to 10% native Polyacrylamide Gel for SYBR® Gold staining (Invitrogen). The following synthetic RNA sequences were used (all from IDT): Repression Domain (RD), UUUGGCUUUUUCCUUUUUGUCUA; Repression Domain star (RD*), UAGAGAAGUAAGGGAAAAUCAAA.

RNase T1 Accessibility Assay. In vitro transcribed RNA was subjected to an annealing protocol as described above. RNA was incubated with RNAse T1 at 37° C. for 15 min. Phenol-chloroform was used to isolate the RNA, followed by isopropanol precipitation. Superscript III Reverse Transcriptase (Invitrogen) was used to synthesize cDNA for 15 min. Pri-miR-17˜92 specific primers was labeled by ³²P-ATP using T4 Polynucleotide Kinase (NEB), and purified by Microspin™ G-50 Columns (GE Healthcare). cDNAs was finally subjected to 15% TBE-Urea Polyacrylamide Gel. Primers were listed in Table 3.

Electron Microscopy. All RNA constructs were transcribed using AmpliScribe T7 High Yield Transcription Kit. Transcribed RNAs were then gel purified with a 8% urea polyacrylamide gel and concentration was quantified using NanoDrop 1000. The purified RNA samples were supplemented with 10 mM sodium cacodylate pH 6.8, then heated up to 90 degrees C. for 30 seconds and slowly cooled down to room temperature. The annealed RNA samples were incubated with 10 mM MgCl2 for 20 min. 2 μl of 200 ng/μl RNA sample was applied to glow discharged carbon-coated grids. Grids were stained with 2% uranyl acetate. The EM micrographs were collected on a Tecnai G² Spirit BioTWIN with Hamamatsu ORCA-HR C4742-95-12HR detector at magnification of 49000×. Image processing and particle picking was performed using EMAN2 (Tang et al., 2007). 500 particles were included for all analysis. Scikit-image was used to measure the diameter and circularity of particles. The results were then plotted using matplotlib.

Co-immunoprecipitation and Western Blots. Two days after transfection of V6.5 ESCs with pFlag-CMV2 vector expressing AGO2, ISY1, CPSF3, or DGCR8 cDNAs, each sample was collected and lysed with NETN buffer as described before (Mori et al., 2014). Anti-Flag M2 Affinity Gel (Sigma-Aldrich) was incubated with cell lysates at 4° C. for 2 h. After two washes with NETN buffer, the beads were added with sample buffer and incubated at 95° C. for 5 min. Finally, the protein samples were analyzed by western blot using α-Flag (Sigma), α-Drosha (Cell Signaling), α-ISY1 (Abcam), α-CPSF3 (Abcam), and α-CPSF2 (Abcam) antibodies.

Immunoprecipitation and q.RT-PCR. HEK293 cells were transfected with pFlag-CMV2 vectors expressing ISY1, CPSF3, or DGCR8. After UV cross-linking, lysates were collected with NETN buffer as described before (Mori et al., 2014). One tenth of each cell lysate was directly used for RNA extraction using Trizol reagent (Invitrogen), and the rest was incubated with Anti-Flag M2 Affinity Gel (Sigma-Aldrich) at 4° C. overnight. Anti-Flag M2 Affinity Gel was then washed five times using NETN buffer and before RNA extraction with Trizol reagent and analysis by q.RT-PCR.

RNA-affinity Purification and Mass Spectrometry. In vitro transcribed cold RNA was conjugated to agarose beads and incubated with whole-cell extract from V6.5 ES cells, and the affinity eluate was subjected to SDS-PAGE followed by Coomassie blue staining. Bands were excised, and subjected to mass spectrometric sequencing as described before (Chang et al., 2013).

Lucierase Reporter Assays. Dgcr8−/− ESCs were co-transfected with psiCHECK™-2 vectors containing mouse pri-miR-17˜92 with the indicated siRNA sequences (Table 1) using Lipofectamine 2000 (Invitrogen). After two days of transfection, cells were collected and Passive Lysis Buffer (Promega) added and incubated at RT for 20 min. Dual-Luciferase® Reporter Assay System (Promega) was used to measure the Renilla and Firefly activity.

TABLE 1
List of siRNAs Used

| Name | Sequence (5′→3′) |
|---|---|
| Mouse ISY1_1 | UCAUGAAGGCCAUCGAUUU |
| Mouse ISY1_2 | GUAAAGAAGUCCCAGGAAAUU |
| Mouse SF3B1 | UCGGAAAGUCAGAGACGUAUU |
| Mouse CPSF2_1 | AGCUAAAGAUGCCGAGUUA |
| Mouse CPSF2_2 | GAAUUAAAUGGGACGAGUA |
| Mouse CPSF3_1 | GUACCAGACCUACGUGAAU |
| Mouse CPSF3_2 | UGAGAAGCGUGAAGAGCGA |
| Mouse PRPF4_1 | CCAAAGCGCCUGACGAUUU |
| Mouse PRPF4_2 | GAUUGUGGAUUGCUAAUUA |
| Mouse SNRNP40_1 | GUGAUAACUAUGCGACGUU |
| Mouse SNRNP40_2 | AGGCUCAGACUGCGGAAUU |
| Mouse U2AF2_1 | CUGCUUGUCCAGAGGGCGA |
| Mouse U2AF2_2 | GGUUAAGGAUAGUGCCACA |
| Mouse CSTF2_1 | GACUGUAACUGGAGACGUA |
| Mouse CSTF2_2 | ACAAAUAUCCCAACGCUGA |
| Mouse CSTF2T_1 | GCGAAACCUCAAUGGGCGA |
| Mouse CSTF2T_2 | GACCUAACGUCAUGUUGAA |
| Mouse FIP1L1_1 | CAAACAAGAUUACGGUACA |
| Mouse FIP1L1_2 | ACAGUGACGAAGAGCGAUA |

mRNA and miRNA by q.RT-PCR. For mRNA analysis, 3 μg total RNA was treated with DNase (Promega) for 2 hr to remove genomic DNA. Superscript III Reverse Transcriptase (Invitrogen) and random primers were used to synthesize cDNA, and IQ SYBR Green Supermix (Bio-Rad) was used to quantify the cDNA. For miRNA analysis, 10 ng total RNA was used. Taqman probes and Universal PCR master mix (Applied Biosystems) were used for cDNA detection. All the primers used for qPCR were listed in Table 4.

TABLE 4
List of Primers Used for q.RT-PCR

| Name | Sequence (5′→3′) |
|---|---|
| Mouse ISY1_F | GTTACCTGGATGAAGATGATGG |
| Mouse ISY1_R | TGGTTGCCTTCCTCATCA |
| Mouse SF3B1_F | CAACTTCCAGTGCTCGTAAA |
| Mouse SF3B1_R | CATTATGTGACCTGGAGTCG |
| Mouse CPSF2_F | TGAAGGGAAAGAACTTGAGG |
| Mouse CPSF2_R | CTTTGTCTTGTGAGCCGAT |
| Mouse CPSF3_F | CAAGACAAGAAGACAGACACCT |
| Mouse CPSF3_R | GAGAGCAAAGACAGGAATGAG |
| Mouse DGCR8_F | TCGGAAGAAACCCAAGATGT |
| Mouse DGCR8_R | GCCACAGGGAAGGAACTTAG |
| Mouse Drosha_F | CCCGACCTATGTAGAGAATCAGAT |
| Mouse Drosha_R | TTGGAGTGGGTGGAGAGGAT |
| Mouse U2AF2_P1F | TGTTTATGTGCCTGGAGTTG |
| Mouse U2AF2_P1R | GGTGTCTGATTGATGGTGCT |
| Mouse U2AF2_P2F | TTCCCAAACAGCCTCTTATC |
| Mouse U2AF2_P2R | GGAAGACTAAGGAGAAGAACCA |
| Mouse CSTF2_P1F | TTCTATGCCAGGTGGAGTTC |
| Mouse CSTF2_P1R | GTCATTGGGAGCATCACCTA |
| Mouse CSTF2_P2F | TCCTGAGCAAAGACAGAGTATC |
| Mouse CSTF2_P2R | GAGCAATGGCGATGTAAGAC |
| Mouse CSTF2F_P1F | TGTGAAGGACATTCCACCTC |
| Mouse CSTF2F_P1R | GTCACTGAGAGCAAAGTCCC |
| Mouse CSTF2F_P2F | TAGTTCCAGAGGTCCGATGA |
| Mouse CSTF2F_P2R | CTTGTGGAGTTACCTGGCTT |
| Mouse FIP1L1_P1F | GCACCTCTTCTCAGTCTCAGA |
| Mouse FIP1L1_P1R | GAGGAGGAAGGTGAGTAGGA |
| Mouse FIP1L1_P2F | GGAAACAAGACACAAGTCCTC |
| Mouse FIP1L1_P2R | GCATCTATTTCTGGGACTGAC |
| Mouse PRPF4_P1F | TGGACCAGAAGGATGTCAAC |
| Mouse PRPF4_P1R | GCAATGTCATACACACCCAT |
| Mouse PRPF4_P2F | CGGAAGGACTCAGAACTTGT |
| Mouse PRPF4_P2R | TGGCAGAACCTGAGATGAGA |
| Mouse SNRNP40_P1F | GACTGTGATAACTATGCGACG |
| Mouse SNRNP40_P1R | CTGGCTGGGTAACAGGAGTT |
| Mouse SNRNP40_P2F | TCCCAAAGAGAGATGTGTGA |
| Mouse SNRNP40_P2R | ACTGGATGCTGAGAGGATGA |
| Mouse pri-miR-17~ | GGTGAGTATATTCTAGTTTTGTA |
| 92_5′Upstram_F | |
| Mouse pri-miR-17~ | TCGACAAGATTAATGATTATTGTT |
| 92_5′Upstram_R | |
| Mouse pri-miR- | AAGGCTTACATGTGTCCAATT |
| 17~92_CS_F | |
| Mouse pri-miR- | CACTTAGGGCAGTAGATGCT |
| 17~92_CS_R | |
| Mouse pri-miR-17~ | TGGTTTGAAGAGTCAGTCCTAC |
| 92_3′Downstram_F | |
| Mouse pri-miR-17~ | AGCACGCACTTCCTGAATA |
| 92_3′Downstram_R | |

### Results

miR-17˜92 Expression is Regulated Posttranscriptionally During ESC Differentiation.

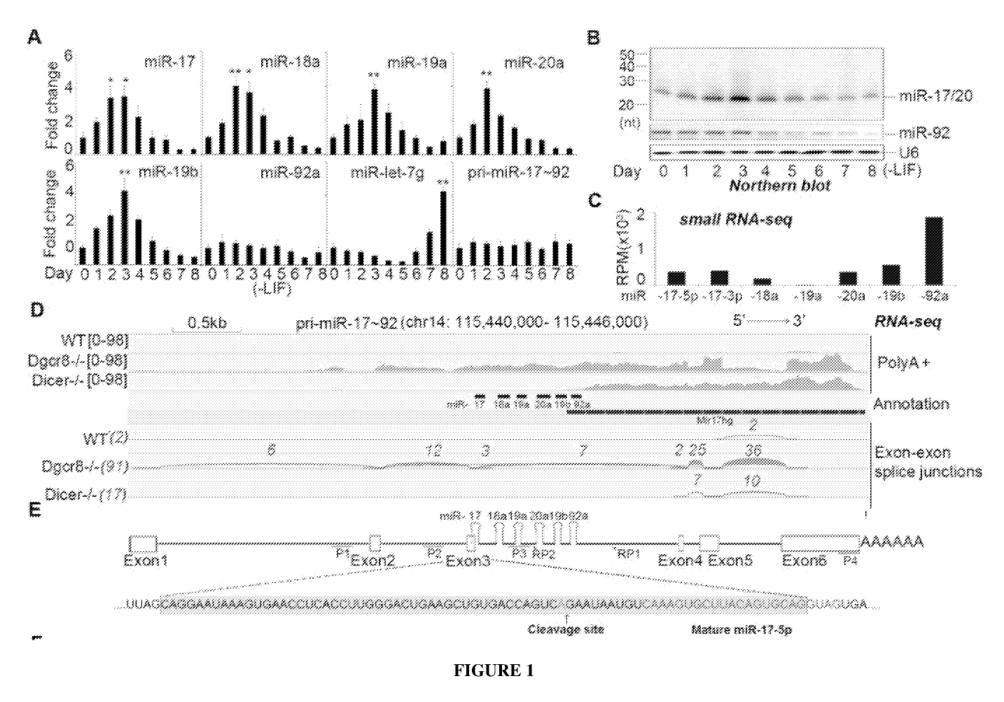

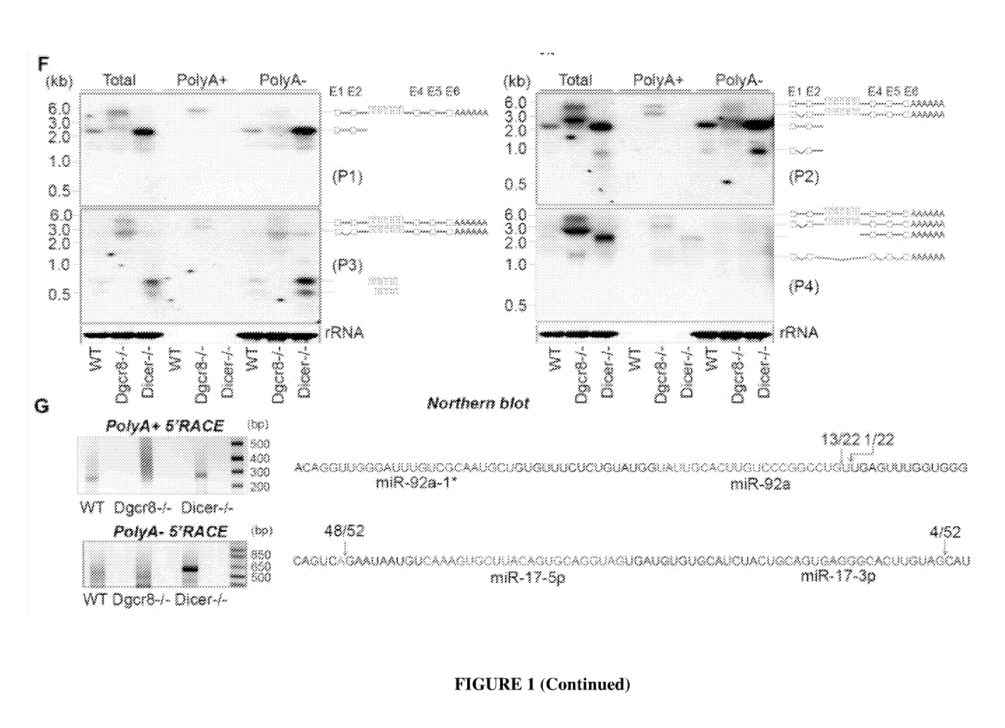

To investigate possible miR-17˜92 regulatory mechanisms, miRNA expression over the course of ESC differentiation was analyzed. As a control, levels of let-7 miRNA that is repressed by Lin28 in ESCs and accumulates during the later stages of cell differentiation were monitored (Viswanathan et al., 2008). This analysis revealed that, while miR-92 expression was relatively constant throughout the differentiation time course and correlated quite well with expression of pri-miR-17˜92, the relative expression of the other miRNAs from this locus was more dynamic with a peak in miR-17, -18a, -19a, -20a, and -19b expression observed around days 2-3 of differentiation, thereby implicating posttranscriptional control mechanism(s) (FIG. 1A, B). To investigate the relative abundance of the different miRNAs from this cluster small RNA cloning and high-throughput cDNA sequencing from mESCs were performed. This analysis revealed a strong predominance of miR-92 sequences compared to the other miRNAs in this cluster (FIG. 1C). Together, these results support that the relative expression of the six miRNAs processed from pri-miR-17˜92 is dynamically regulated during ESC differentiation. The possible mechanisms for this developmentally regulated, posttranscriptional control of miR-17˜92 were next investigated. As a first step, the pri-miR-17˜92 sequence was defined, using RNA cloning and high-throughput cDNA sequencing from ESCs. Since most pri-miRNAs are present at very low levels in steady state RNA likely owing to their cleavage by the Microprocessor, Dgcr8 (and Dicer) knockout ESCs were included in this analysis. The sequencing data from Dgcr8 knockout ESCs indicated that the mouse pri-miR-17˜92 gene spans more than 5 kilobases (kb) and contains multiple introns. The miRNA sequences themselves are located within Intron 3 of the host transcript, similar to the annotated human gene (FIG. 1D, E). As expected, more sequences mapping to pri-miR-17˜92 were detected in the Dgcr8 knockout compared to the control ESCs. The increased read abundance extended across the entire cluster in Dgcr8 knockout compared to the wild type cells suggesting that this region contains the Microprocessor substrate RNA that is stabilized in Dgcr8-deficient cells. In contrast, an increased number of reads specifically mapping downstream of the miRNA cluster in the Dicer knockout ESCs were detected. This polyadenylated 3′ RNA fragment likely represents a product of Microprocessor-mediated cleavage of pre-miR-92. The elevated abundance of this 3′ RNA fragment in Dicer knockout cells was confirmed by quantitative reverse transcription PCR (q.RT-PCR) (data not shown) and is consistent with a feedback mechanism leading to increased pri-miR-17˜92 expression in the absence of the functional mature miRNAs. Altogether these sequencing results reveal that miR-17˜92 miRNAs are embedded in a long, spliced, and polyadenylated pri-miRNA expressed from a locus of >5 kb. Furthermore the developmental dynamics indicate that the processing and/or stability of the mature miRNAs expressed from this cluster can be differentially regulated.

Identification of a Processed pri-miR-17˜92 Intermediate.

To confirm the sequencing results and to obtain additional experimental validation of the pri-miRNA, Northern blots with probes spanning the locus were performed (FIG. 1E). Total RNA, as well as RNA separated into the PolyA+ and PolyA− fractions prepared from wild-type, Dgcr8−, and Dicer-knockout mESCs were included. A large (>5 kb) transcript was detected in the Dgcr8 knockout RNA samples in both total RNA as well as PolyA+ RNA with all probes (P1-4) tested. This likely corresponds to the full-length primary transcript (FIG. 1F) and supports the RNA sequencing results. This analysis also identified (with probes P1 and P2) an additional prominent band of ˜2.5 kb that was detected in the total and PolyA− RNAs from wild-type and Dicer−/− ESCs that corresponds to a 5′ RNA fragment containing Introns 1 and 2 (and likely also Exons 1 and 2). Strikingly, probe 3 (P3), that spans the miRNA sequences in Intron 3, detected a predominant band of ˜800 nt in the total and PolyA− RNAs (FIG. 1F). Finally, a probe complementary to sequences in the 3′ region detected ˜2.2 kb band only in the total, and PolyA+ RNA and not in the PolyA− RNA from Dicer−/− cells. Altogether, these Northern blot results support that the polyadenylated pri-miRNA, detectable in Dgcr8−/− cells, is >5 kb, and that this pri-miR-17˜92 is cleaved into three major fragments: ˜2.5 kb 5′ region, ˜800 nt region containing miRNA sequences, and ˜2.2 kb 3′ region with a PolyA tail.

To further explore the possibility that these RNA fragments correspond to specific cleavages of the pri-miR-17˜92 and to map with nucleotide resolution the cleavage sites, 5′ Rapid Amplification of cDNA ends (5′ RACE) was performed using the indicated primers (FIG. 1G). This analysis revealed that the majority of the 5′ ends of the polyadenylated 3′ region map to the expected Drosha cleavage site for the biogenesis of miR-92a with the remainder of reads corresponding to Drosha cleavage of pre-miR-19b. However, using a primer that is complementary to sequences within the miRNA cluster, it was unexpectedly found that the 5′ cleavage site occurs 9 nt upstream of the miR-17-5p sequence and is inconsistent with the expected Drosha cleavage. Taken together these results indicate that the pri-miR-17˜92 is specifically cleaved close to the pre-miR-17 hairpin by an unknown nuclease to release a 5′ upstream RNA fragment and that Drosha processing of pre-miR-92 generates the 3′ cleavage to liberate a ‘progenitor-miRNA’ (pro-miRNA) intermediate containing miR-17, -18a, -19a, 20a, and -19b.

The 5′ Fragment of pri-miR-17˜92 Inhibits Microprocessor Activity.

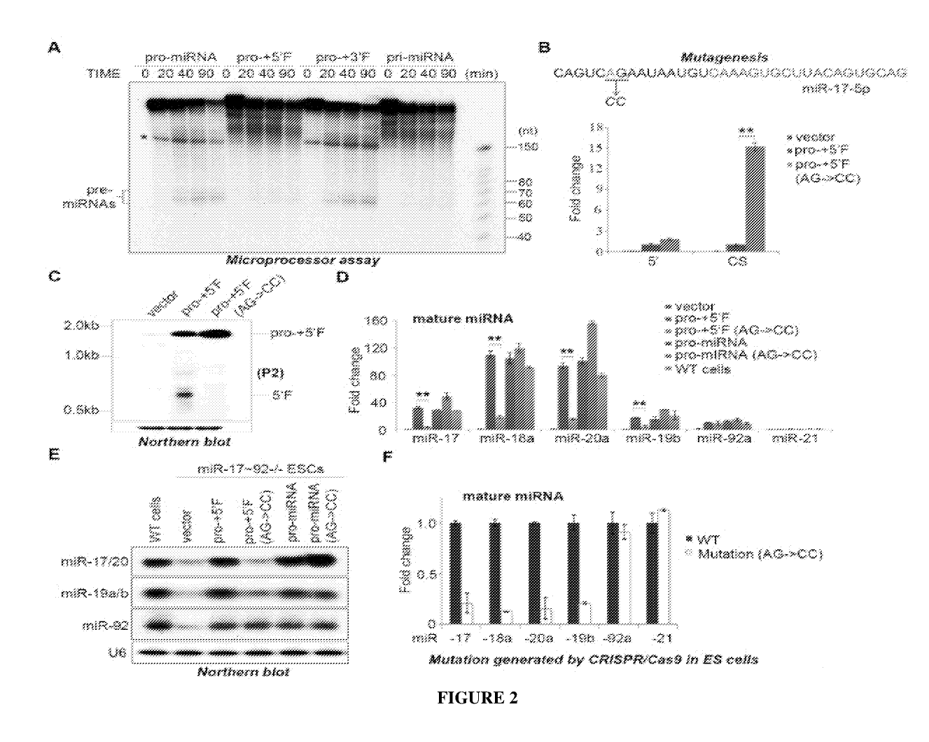

To examine whether the pro-miRNA might represent a miRNA biogenesis intermediate, different RNA substrates were tested in Microprocessor assays. The pri-miRNA sequence used in these experiments corresponds to a genomic DNA sequence beginning at the 5′ end of Exon 2 and ending at the 3′ end of Exon 6 (FIG. 1E). The pro-miRNA starts at the 5′ side of pre-miR-17 and ends ˜50 nt downstream of the 3′ end of pre-miR-92. The pro-miRNA+5′F and pro-miRNA+3′F include the pro-miRNA with the additional upstream or downstream sequences present in the pri-miRNA, respectively. These processing assays performed with in vitro transcribed RNAs and affinity purified Microprocessor complex revealed that pro-miRNA is a preferential Microprocessor substrate compared to pri-miRNA. Moreover, it was found that the 5′ region of the pri-miR-17˜92 dramatically inhibited processing of pre-miRNAs by the Microprocessor (FIG. 2A).

Cleavage of pri-miR-17˜92 to pro-miRNA is a Key Step in miRNA Maturation.

To explore the functional impact of pri-miRNA cleavage to the pro-miRNA biogenesis intermediate, rescue experiments in mouse ESCs in which the endogenous miR-17˜92 is deleted were performed. miR-17˜92 knockout ESCs were transfected with plasmids expressing either the wild-type pri-miR-17˜92 or a mutant version in which two nucleotides (AG to CC mutation) at the potential cleavage site were mutated. q.RT-PCR analysis indicated that both plasmids produced similar levels of pri-miRNA transcript in transfected ESCs (FIG. 2B) yet when PCR primers spanning the cleavage site were used a strong accumulation of the uncleaved RNA was detected supporting that the mutation inhibits pri-miRNA cleavage (FIG. 2B). Northern blot analysis detected a cleaved 5′ fragment in cells expressing the wild type but not the mutant pri-miR-17˜92 plasmid (FIG. 2C). The functional impact of this cleavage site mutation on mature miRNA biogenesis was next examined. Analysis of miRNA expression by q.RT-PCR and by Northern blot in these rescue experiments revealed that the AG-CC mutation inhibits expression all miRNAs in the cluster except for miR-92 (FIG. 2D, E). Since the plasmid expressing pro-miR-17˜92 also contains a small amount of upstream sequence that includes the cleavage site the effect of the same AG-CC mutation could also be tested in this context. The AG-CC mutation had no effect on miRNA biogenesis expressed from the pro-miR-17˜92 plasmid and therefore was specifically required to selectively license Microprocessor-mediated production of pre-miR-17, -18a, -19a, 20a, and -19b, from the pri-miR-17˜92 (FIG. 2D, E). Next, to examine whether pro-miRNA biogenesis is an important intermediate step for the expression of endogenous miRNAs, CRISPR/Cas9 technology was used to engineer the AG-CC mutation at the pri-miR-17˜92 locus in ESCs. Introduction of this mutation led to dramatically diminished expression of miR-17, -18a, -19a, 20a, and -19b compared to wild type cells but had no effect on endogenous miR-92 expression (or an unrelated control miRNA, miR-21) (FIG. 2F). These results suggest that the 5′ region of pri-miR-17˜92 inhibits production of most miRNAs in this cluster except for miR-92 and that this autoinhibitory mechanism might explain the posttranscriptional regulation of pri-miR-17˜92 expression that was observed in ESCs (FIG. 1A).

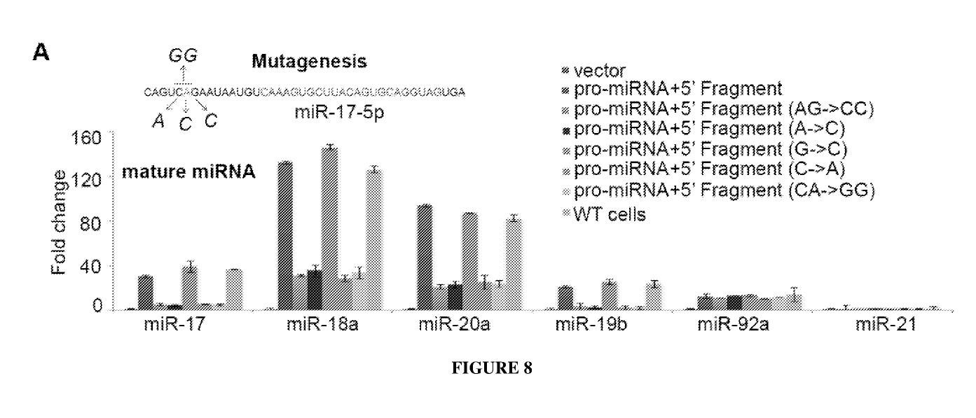

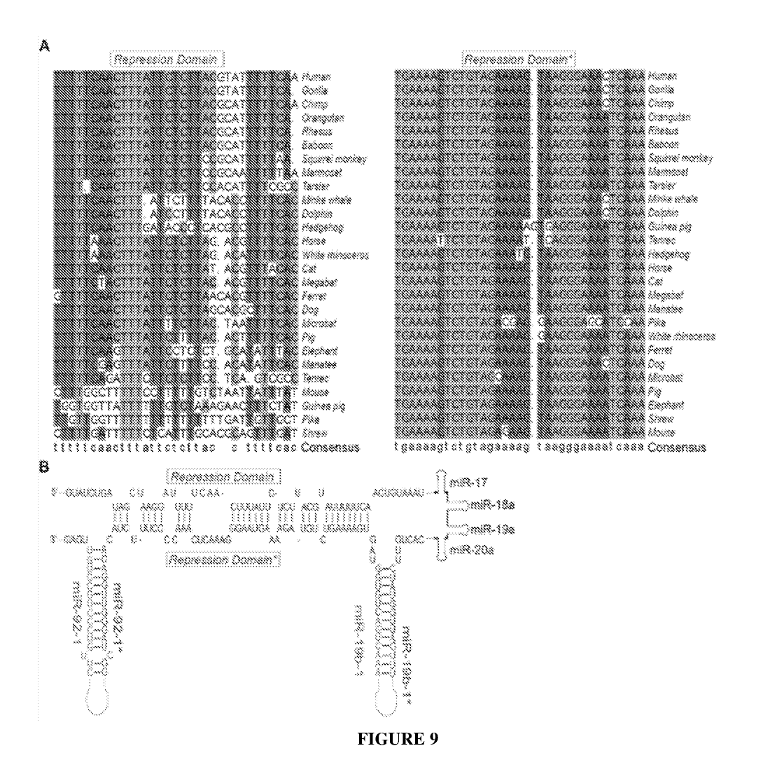

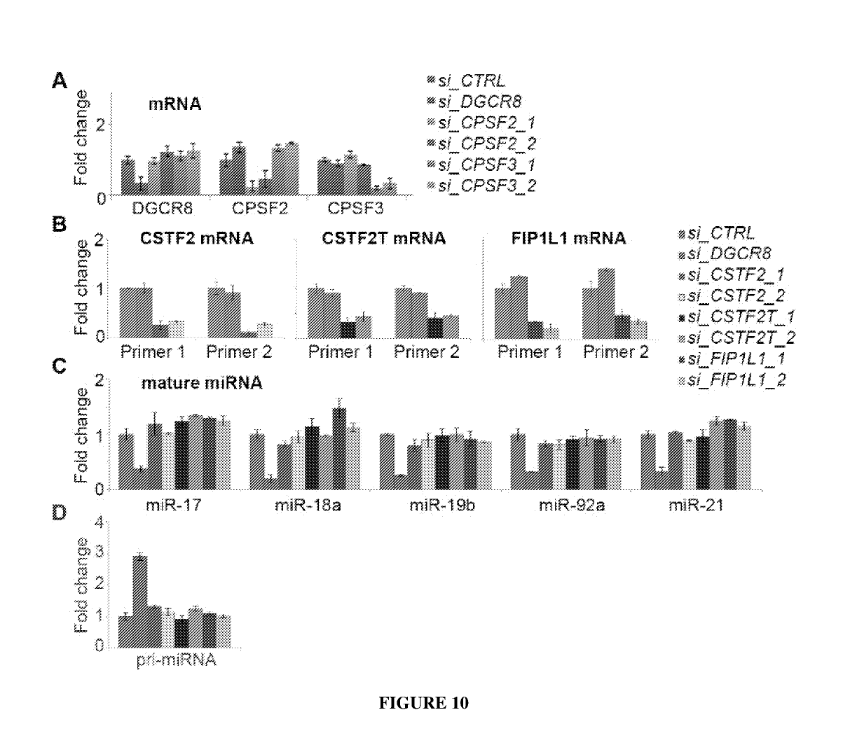

Since the 5′ RACE strategy employed could not accurately distinguish whether cleavage occurred after the A or the G nucleotides in the pri-miR-17˜92 (due to the dCTP 3′ tailing of the cDNA with terminal deoxynucleotidyl transferase and use of complementary oligo-G containing PCR primer), additional mutagenesis at the cleavage site was performed and the effects on miRNA expression were examined in rescue experiments. This revealed that mutation of the G nucleotide had no impact on miR-17˜92 whereas the A mutation dramatically suppressed miRNA expression comparable to the AG mutant. Furthermore mutation of the preceding C nucleotide similarly selectively inhibited miRNA biogenesis (FIG. 8). Together, these results demonstrate that cleavage of the autoinhibitory 5′ RNA fragment to generate pro-miRNA is an obligate step for the biogenesis of miRNAs from the pri-miR-17˜92.

Identification of Two Complementary Repression Domains that Control miRNA Biogenesis.

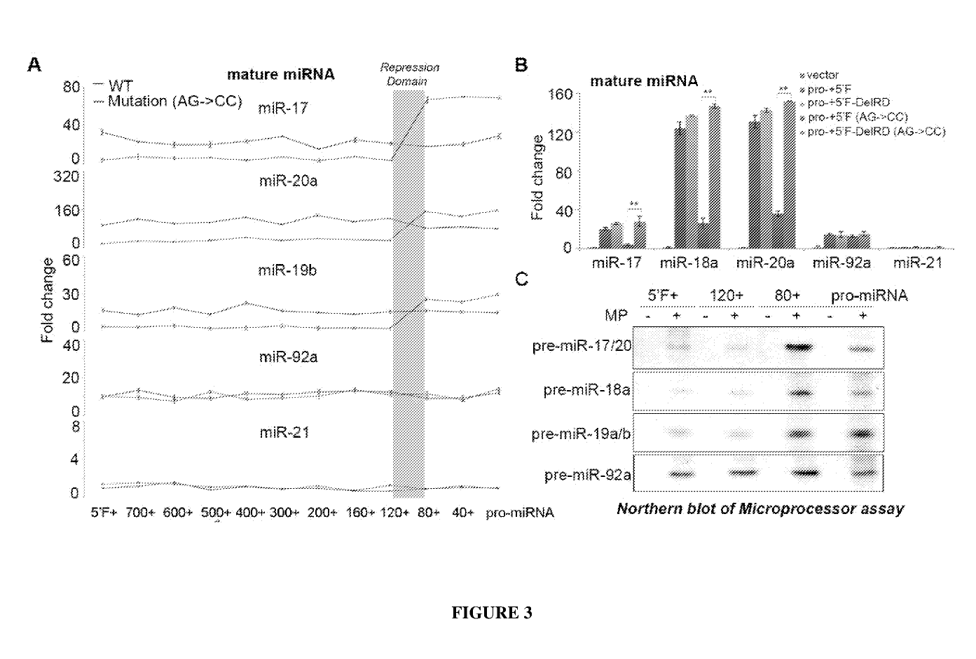

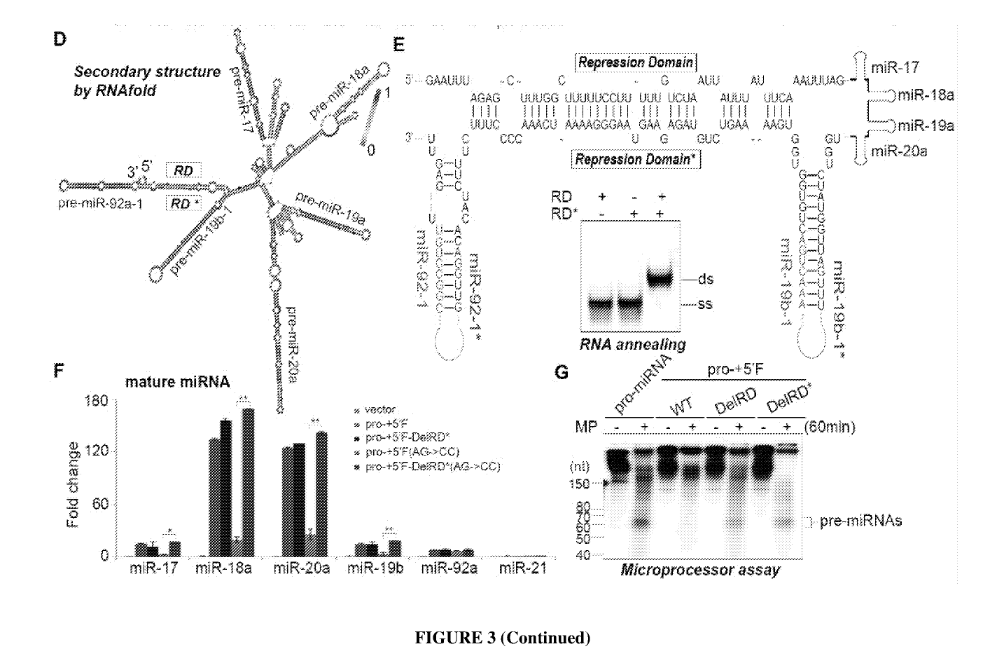

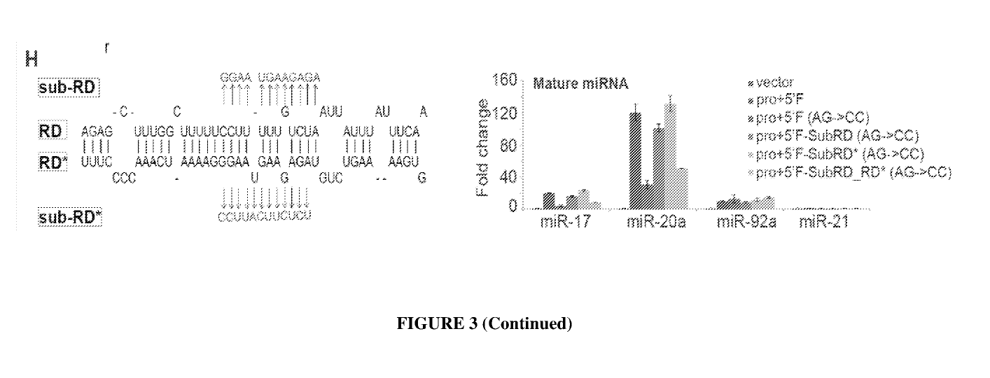

Next, to precisely define the cis-regulatory RNA sequences present in the 5′ fragment of pri-miR-17˜92, additional genetic rescue experiments were performed using a panel of pri-miR-17˜92 expression constructs in which portions of the 5′ inhibitory fragment are deleted. q.RT-PCR analysis of transfected (pri-miR-17˜92−/−) ESCs revealed that a ˜40 nt domain located ˜80-120 nt upstream of the identified cleavage site is responsible for the selective repression of miRNA expression (FIG. 3A). This was further confirmed by similar experiments in which only the 40 nt repression domain (RD) was deleted in the context of the pro-miR-17˜92 containing the 5′ fragment (FIG. 3B). To further examine the functional impact of the repression domain, in vitro Microprocessor assays were performed. To distinguish processing of the individual pre-miRNAs from the cluster, Microprocessor assays were performed using non-radiolabeled substrate RNAs and visualized pre-miRNAs by Northern blot analysis. This confirmed that the ˜40 nt repression domain (RD) within the 5′ region of pri-miR-17˜92 selectively inhibits the processing of pre-miR-17, -18, -19, and -20 from the cluster but has no inhibitory effect on the processing of pre-miR-92 (FIG. 3C). Altogether these results suggest that the RD in the 5′ region of pri-miR-17˜92 inhibits production of most miRNAs in this cluster except for miR-92 and that this autoinhibitory mechanism might explain the posttranscriptional regulation of pri-miR-17˜92 expression.

It was postulated that the RD might impact the secondary structure of this pri-miRNA cluster to suppress Microprocessor activity. The secondary structure of pri-miR-17˜92 containing the minimal RD was computationally predicted using the RNAFold algorithm (rna.tbi.univie.ac.at/cgi-bin/RNAfold.cgi). This indicated that the RD might base-pair with a highly conserved sequence that was termed repression domain* (RD*) that is located between pre-miR-19b and pre-miR-92 (FIG. 3D, E, 9). This was intriguing since this complementary RD* is located at the boundary of the miRNAs whose processing is suppressed and the most 3′ miR-92 that escapes repression. The ability of RD and RD* sequences to form a duplex was experimentally confirmed in vitro (FIG. 3E), and genetic rescue experiments with plasmids lacking the RD* revealed the functional requirements of this region in miRNA repression with results comparable to those obtained with constructs lacking the RD (FIG. 3B and FIG. 3F). Indeed, deletion of the RD or the RD* had a similar effect on Microprocessor activity indicating that both domains are important for the autoinhibition of miRNA biogenesis (FIG. 3G). To test the present model that base-paring between the RD and RD* is important for miRNA regulation, expression plasmids with either the RD or RD* mutated were generated. Individual mutations of RD or RD* led to elevated miRNA expression whereas combining the compensatory mutations that restored base-pairing between RD and RD* reestablished the autoinhibition of miRNA expression in transfected cells (FIG. 3H).

pri-miR-17˜92 Adopts an RNA Conformation that Inhibits Microprocessor.

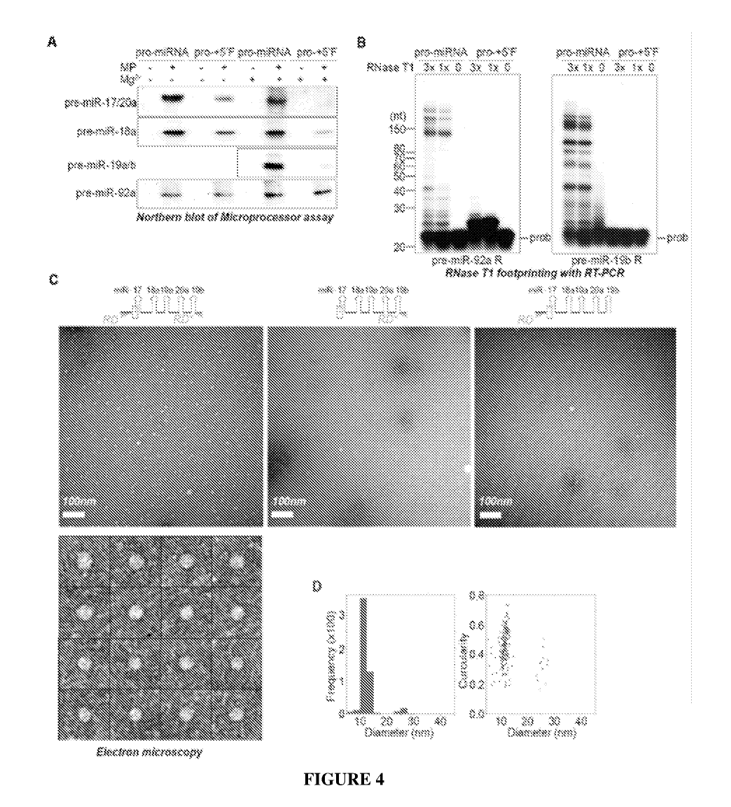

To gain insight into the mechanism by which pri-miR-17˜92 processing might be regulated, the possible involvement of RNA conformational changes mediated by the RD and RD* were tested. The extent of selective Microprocessor inhibition was maximized by RNA annealing in the presence of MgCl₂—a result that further supports that the repressive effect of the 5′ region likely involves an RNA conformational change (FIG. 4A). The differential RNAse T1 accessibility of pro-miR-17˜92 with or without the 5′ fragment was analyzed next. This analysis revealed that the 5′ fragment confers striking resistance to nuclease digestion, further supporting that the pro-miR-17˜92 containing the 5′ fragment adopts a compacted conformation (FIG. 4B). To gain direct evidence that the pri-miRNA containing the 5′ RD adopts a distinct conformation, electron microscopy was used to directly analyze the higher order structure of different RNAs. This analysis revealed that a pri-miR-17˜92 fragment containing both the RD and RD* forms highly compacted, circular particles ˜12 nM in diameter whereas RNAs lacking either the RD or the RD* did not form particles under the same conditions (FIG. 4C-D). Altogether, these results uncover an important role for the RD and the RD* in the dynamic control of pri-miR-17˜92 biogenesis through the formation of a compacted RNA conformation that is refractory to cleavage by Microprocessor.

The CPSF3 Endonuclease is Required for pro-miRNA Biogenesis and miRNA Expression.

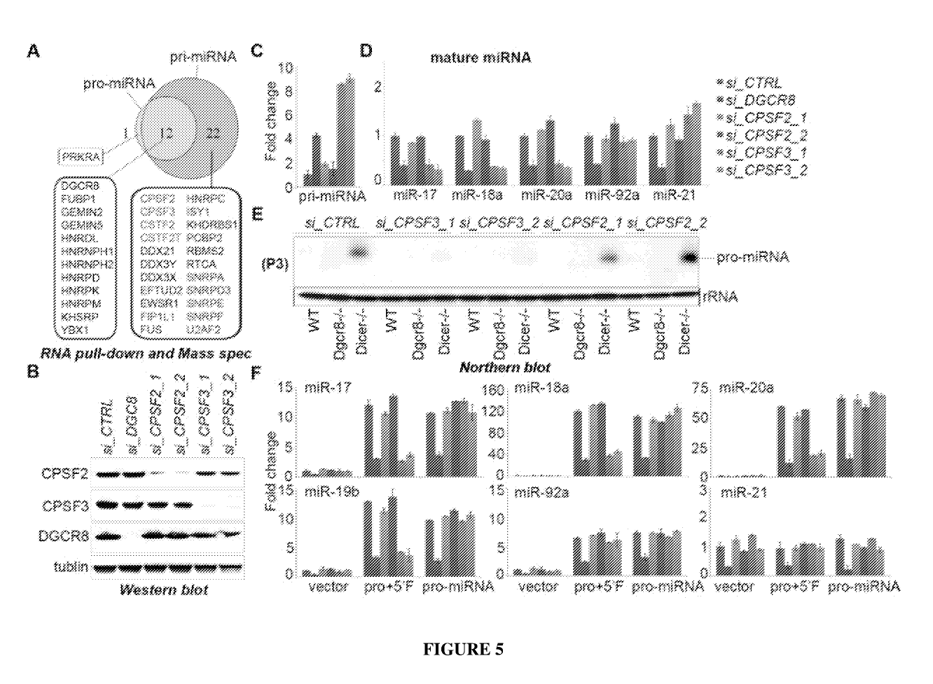

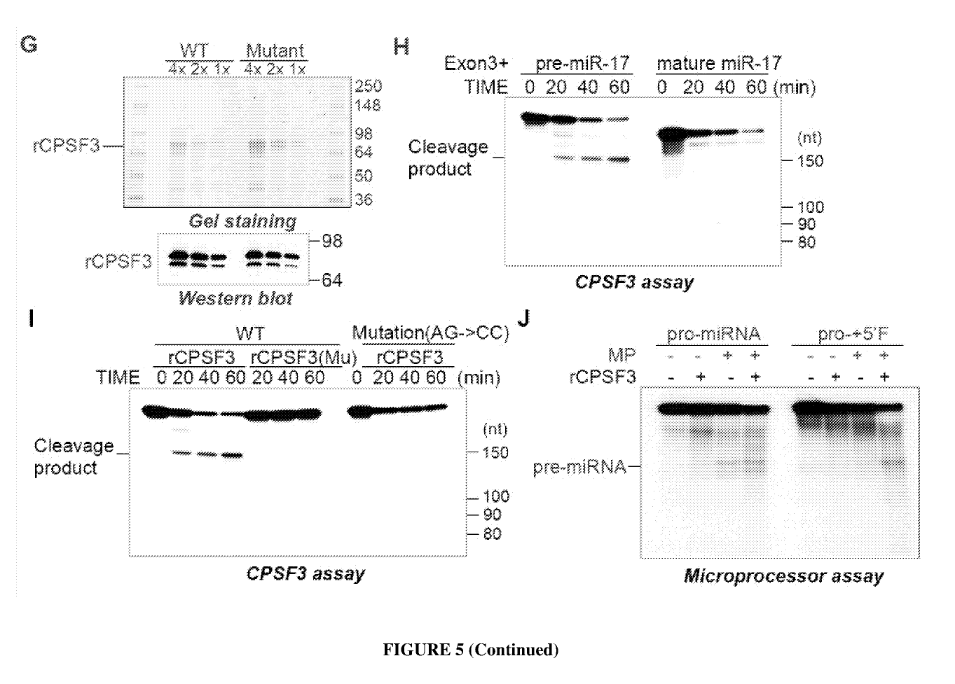

To identify protein factors that might be involved in pro-miRNA biogenesis and the posttranscriptional regulation of miR-17˜92 expression, RNA affinity purifications and mass spectrometric protein identification were performed. pri-miR-17˜92 and pro-miR-17˜92 RNA sequences were in vitro transcribed, covalently coupled to agarose beads, and incubated with extracts prepared from mouse ESCs. Several RNA-binding proteins including DGCR8 were identified in both RNA purifications. However, several proteins were found exclusively in the pri-miR-17˜92 purification. The majority of the identified proteins fall into two main categories; factors involved in pre-mRNA 3′ end cleavage, and splicing regulators (FIG. 5A). Since the Cleavage and Polyadenylation Specificity Factor complex possesses ribonuclease activity, the possible role of components of this complex in miR-17˜92 biogenesis was initially examined. siRNAs were used to knockdown CPSF2 (also known as CPSF-100), CPSF3 (also known as CPSF-73), CSTF2 (CstF-64), CSTF2T (TCstF-64), or FIP1L1 in ESCs and examined the effects on mature miRNA expression. This analysis revealed that CPSF3, but not CPSF2 or other mRNA cleavage/polyadenylation factors tested, is specifically required for expression of all the miRNAs in the cluster except for miR-92 (FIG. 5B-D, 10). As a positive control, DGCR8 was depleted and it was found that expression of all miRNA tested was decreased as expected. Moreover, Northern blot analysis revealed that CPSF3 (but not CPSF2) is required for pro-miR-17˜92 biogenesis (FIG. 5E). These experiments were performed in Dicer−/− ESCs since the level of pri-miR-17˜92 is elevated in these cells. Genetic rescue of miR-17˜92−/− ESCs with miRNA expression plasmids revealed that CPSF3 is specifically required for miRNA expression from the pri-miRNA containing the 5′ region and not from the pro-miRNA (FIG. 5F). Considering the established role of CPSF3 as the endonuclease responsible for the cleavage of the 3′ end of both pre-mRNA and histone mRNA, as well as the known CPSF3-mediated cleavage at ‘CA’ dinucleotides, it was hypothesized that CPSF3 might be the endonuclease that cleaves pri-miRNA-17˜92 to remove the RD and license Microprocessor activity (Dominski et al., 2005; Mandel et al., 2006). To directly test this, recombinant CPSF3 (rCPSF3), and a catalytic mutant (D75K/H76A) version of CPSF3 purified from E. coli was generated (FIG. 5G). Cleavage assays using different RNA substrates were performed. It was found that a fragment of pri-miR-17˜92 containing the 5′ repression domain, cleavage site, and the entire pre-miR-17 sequence is specifically cleaved by rCPSF3 whereas a slightly truncated RNA that lacks the pre-miR-17 stem loop was not an efficient substrate (FIG. 5H). Moreover, a 2-nucleotide mutation (AG-CC) at the cleavage site abolished the CPSF3-mediated pri-miRNA cleavage, and the catalytic mutant CPSF3 was inactive in these assays (FIG. 5I). Furthermore, it was found that addition of rCPSF3 to Microprocessor assays could relieve the inhibition mediated by the 5′ fragment of pri-miR-17˜92 (FIG. 5J). Altogether these data strongly support the present model whereby CPSF3 is the nuclease responsible for specific pri-miRNA cleavage to remove the repression domain and license Microprocessor-mediated production of pre-miRNA from this cluster.

Spliceosome Subunits are Required for pro-miRNA Biogenesis and miRNA Expression.

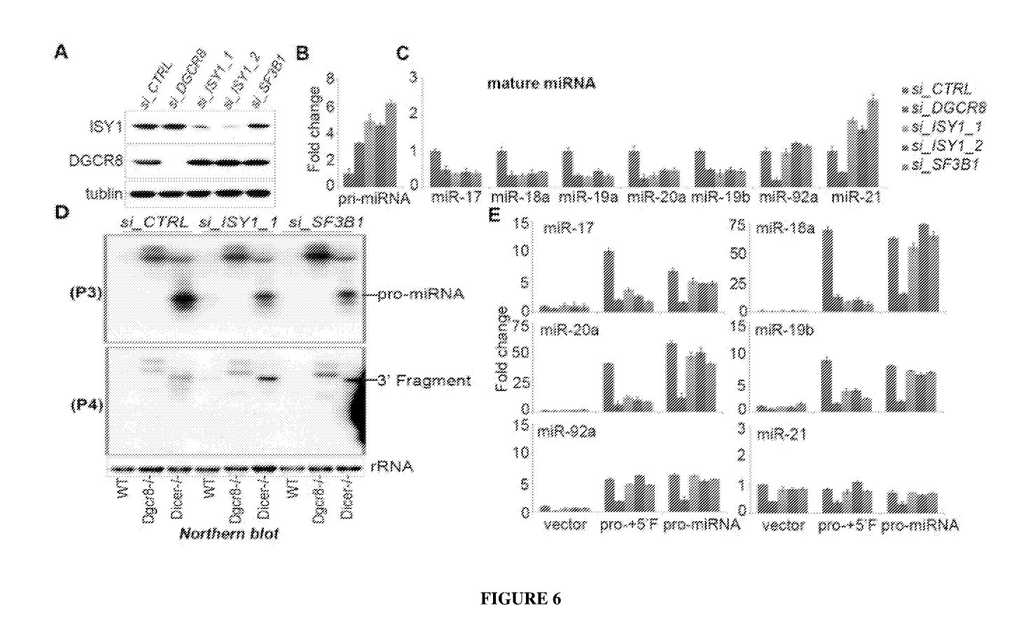

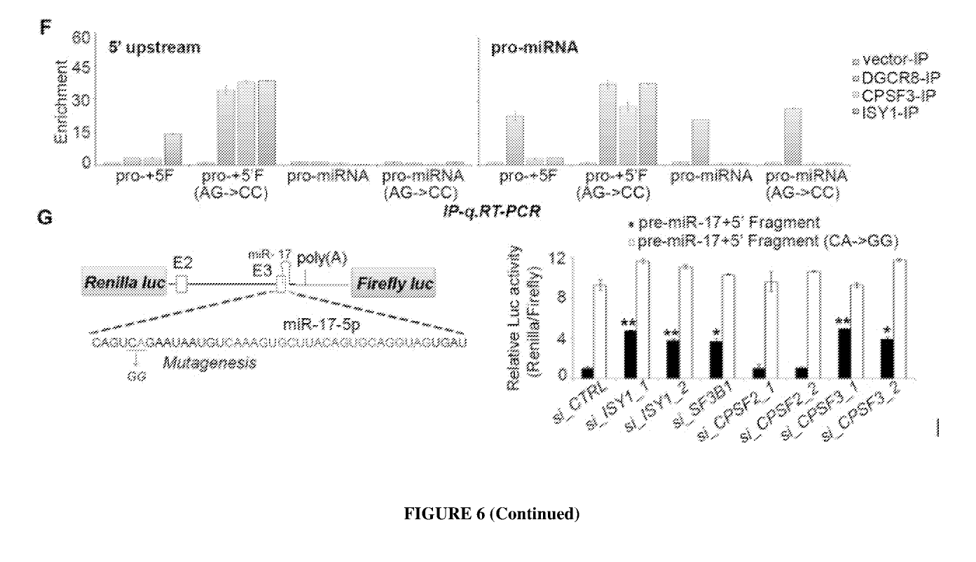

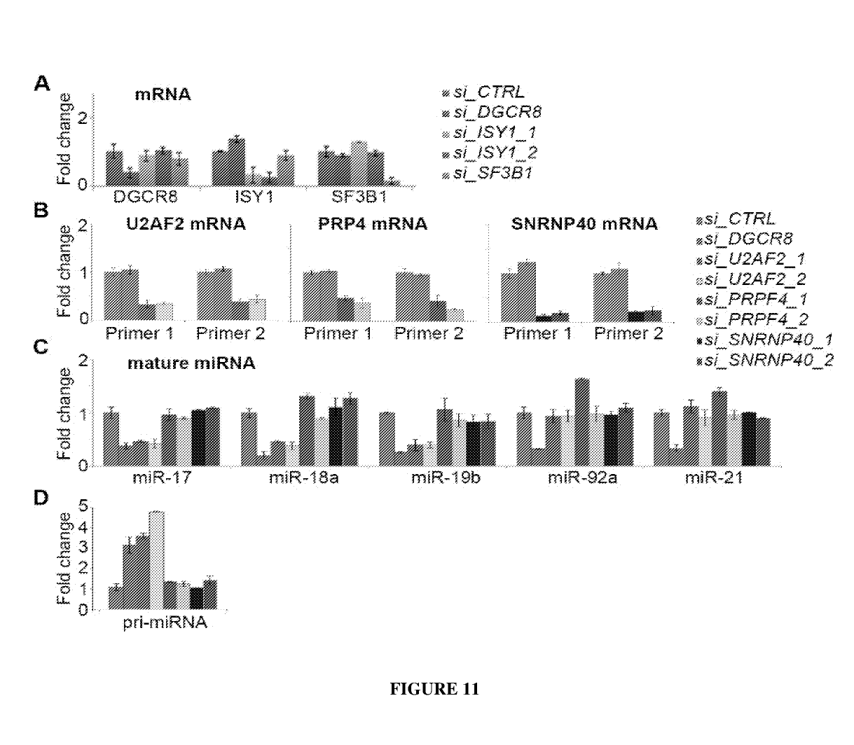

Considering the mass spec data as well as a previous report that found that processing the 3′ end of histone pre-mRNAs by CPSF3 requires components of the U7 snRNP, whether certain spliceosome subunits might help recruit the CPSF3 endonuclease activity to pri-miR-17˜92 in vivo was next examined (Dominski et al., 2005). The role of ISY1, a poorly characterized homolog of the non-essential Isy1p protein in yeast was initially examined. Isy1p is a subunit of the NineTeen Complex, and is involved in the first step of splicing to control splicing fidelity (Dix et al., 1999; Villa and Guthrie, 2005). SF3B1, a component of the U2 small nuclear ribonucleoprotein complex (U2 snRNP) that, although not identified in the mass spectrometric analysis of pri-miR-17˜92 associated proteins, is a much more well characterized splicing factor and was subsequently added to the characterization. siRNAs were used to individually knockdown ISY1, and SF3B1 in ESCs and the effects on miRNA expression were examined (FIG. 6A-C). This revealed that depletion of ISY1 or SF3B1 led to diminished expression of all miRNAs in the pri-miR-17˜92 cluster with the exception of miR-92. Also, expression of miR-21 was not diminished by ISY1, or SF3B1 depletion in these experiments. The accumulation of the pri-miR-17˜92 upon depletion of ISY1, SF3B1, and DGCR8 was observed, consistent with a role of these factors in the miRNA biogenesis pathway (FIG. 6B). Next, to test whether these splicing factors might be involved in pro-miRNA biogenesis, the relative levels of pro-miRNA were examined by Northern blot in RNAs extracted from ISY1-, and SF3B1-depleted cells. A substantial and specific decrease in pro-miRNA was observed upon ISY1, or SF3B1 knockdown thereby supporting the involvement of these factors in pro-miRNA biogenesis (FIG. 6D). RNAi knockdown of multiple additional spliceosomal factors revealed a specific requirement for ISY1 as well as U2 snRNP components (SF3B1 and U2AF2), but not other splicing factors associated with the second step of splicing including PRPF4 (U4/U6 snRNP) and SNRNP40 (U5 snRNP (FIG. 11). These findings help clarify the requirement of certain splicing factors and strongly support the present model that ISY1 together with ISY1 and the U2 snRNP are specifically required for pro-miRNA biogenesis.

To provide more evidence that splicing factors are selectively required for miR-17˜92 expression, rescue experiments were performed in miR-17˜92 knockout ESCs. It was found that, whereas DGCR8 was required for expression of miRNAs from both the pro-miR-17˜92 as well as the plasmid containing pro-miR-17˜92 with the upstream sequences (pro+5′F), the splicing factors ISY1, and SF3B1 were specifically required for the expression of miRNAs from pro+5′F since depletion of these factors had no effect on expression of the miRNAs expressed from the pro- plasmid (FIG. 6E). Moreover, expression of miR-92 from either plasmid was unaffected by depletion of ISY1, SF3B1 in these rescue experiments. These results indicate the selective requirement of ISY1, and SF3B1 for the expression of miRNAs from the miR-17˜92 locus is likely restricted to the genesis of pro-miRNA.

To further confirm the role of these factors in pro-miRNA biogenesis, affinity purification of DGCR8, CPSF3, and ISY1 containing ribonucleoprotein complexes from cells was performed and the associated RNA was analyzed by q.RT-PCR. For these experiments, cells were co-transfected with plasmids expressing the indicated Flag-tagged protein together with plasmids expressing either wild-type pri-miR-17˜92, the cleavage site mutant version of pri-miR-17˜92, or the corresponding pro-miRNAs. This revealed that unlike DGCR8 that associates with all the RNAs tested, CPSF3 and ISY1 specifically associate with the cleavage site mutant pri-miR-17˜92, consistent with the specific role of these factors in pro-miRNA biogenesis (FIG. 6F).

A Luciferase reporter containing the 5′ region of pri-miR-17˜92 was generated. pri-miR-17˜92 sequences (beginning from the start of exon 2 and ending in the pre-miR-17 hairpin) were cloned into the 3′UTR of the Renilla Luciferase gene (FIG. 6G). A similar approach was previously used to monitor Microprocessor activity (Mori et al., 2014). Cleavage of the 5′ region of pri-miR-17˜92 is expected to destabilize the Renilla luciferase mRNA and lead to decreased Renilla luminescence relative to a control Firefly luciferase. This reporter and a reporter containing a mutated cleavage site were used to examine the effects of ISY1, SF3B1, CPSF2, and CPSF3 knockdown on the relative luciferase values. For factors involved in cleavage of the 5′ region of pri-miR-17˜92, a stabilization of the Renilla luciferase upon knockdown was expected. It was found that depletion of ISY1, SF3B1, and CPSF3, but not CPSF2, led to increased Renilla luciferase relative to the control Firefly luciferase. Importantly, this effect was specific to the wild type pri-miR-17˜92 fragment since depletion of these same factors had no stabilizing effect on the cleavage site mutant reporter. Taken together, the data support that spliceosome components, and the CPSF3 endonuclease subunit of the pre-mRNA 3′ processing complex, are specifically required for cleavage of the pri-miRNA and that multiple factors might cooperate to precisely cleave the 5′ fragment of the pri-miR-17˜92 to generate the pro-miRNA biogenesis intermediate and release the autoinhibition of miR-17, -18a, -19a, 20a, and -19b processing by Microprocessor.

Pro-miRNA Biogenesis Controls miR-17˜92 Expression in Embryonic Stem Cells.

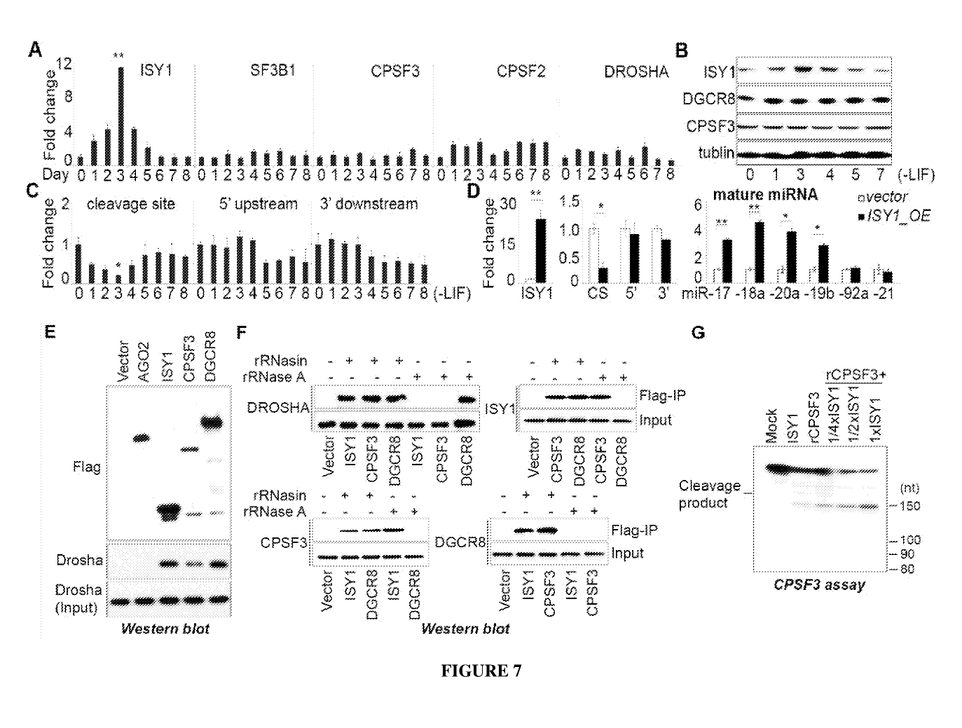

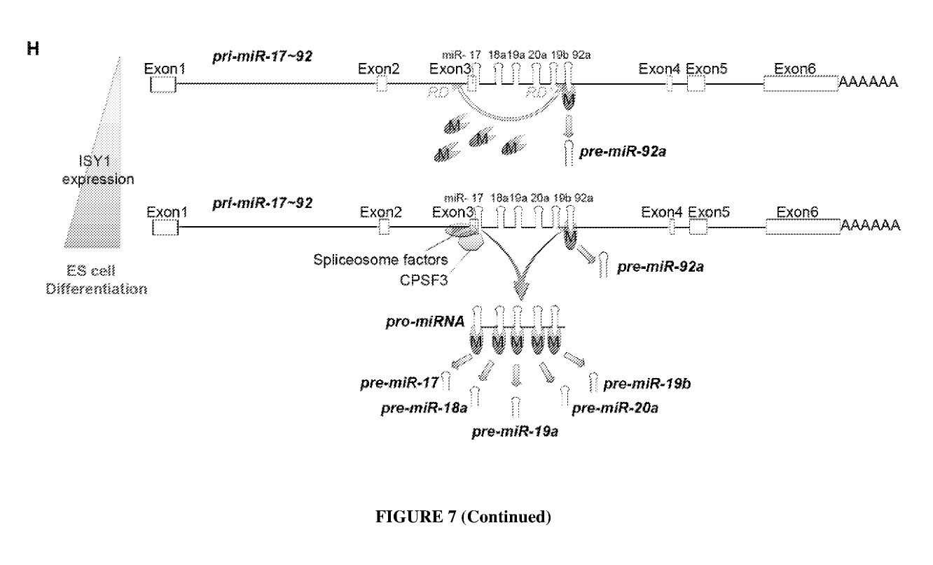

It was considered that developmentally regulated expression of some of these factors required for pri-miR-17˜92 processing might be responsible for the dynamic miRNA expression patterns observed from this cluster during ESC differentiation (FIG. 1A, B). To address this, q.RT-PCR was performed to measure the relative expression of the genes that had been previously identified to play a role in pro-miRNA biogenesis over the differentiation time course. ISY1 expression was thus found to be correlated with miRNA expression with a peak at day 3 of ESC differentiation (FIG. 7A, B). Furthermore this peak in ISY1 expression also correlated with cleavage of the 5′ region of pri-miR-17˜92 since primers spanning the cleavage site (but not other regions of pri-miR-17˜92) showed a decline in signal by q.RT-PCR at day 3 (FIG. 7C). These correlative data suggest that ISY1 might be a limiting factor in ESCs for the processing of certain miRNAs from the pri-miR-17˜92. To directly test this, ISY1 was overexpressed in ESCs and the effects on miRNA expression were measured. It was found that ISY1 overexpression in this context caused a selective miRNA upregulation and corresponding increase in cleaved pri-miR-17˜92 (FIG. 7D). These results indicate that the developmentally regulated generation of pro-miRNA is likely responsible for the posttranscriptional control of miR-17˜92 expression in ESCs.

Considering the developmental requirement of ISY1 for miRNA expression and the involvement of both ISY1 and CPSF3 in pro-miRNA biogenesis, the possible physical and functional interaction between ISY1, CPSF3, and the Microprocessor was next examined. ISY1 and CPSF3 were found to specifically associate with Drosha and DGCR8 in co-immunoprecipitation experiments (FIG. 7E-F). Whereas this interaction with Microprocessor was strongly diminished by RNase treatment, the interaction between ISY1 and CPSF3 complexes was unaffected by RNase and likely therefore not mediated by RNA (FIG. 7F). Functionally, it was found that the addition of immunopurified Flag-ISY1 complex could enhance the specific CPSF3-catalyzed pri-miRNA cleavage in vitro (FIG. 7G). Altogether, these data strongly support the present model whereby CPSF3 is the nuclease that cleaves pri-miRNA to remove the repression domain and license Microprocessor-mediated production of pre-miRNA from this cluster and that this activity is enhanced by the ISY1 complex.

### Discussion

A novel paradigm for miRNA expression control has been uncovered. While proteins that inhibit or promote the biogenesis of certain miRNAs are known, the discovery that cis-acting sequences within the pri-miRNA can selectively and dynamically regulate expression of a miRNA cluster through the formation of an inhibitory RNA conformation reveals an additional posttranscriptional regulatory mechanism for the precise control of miRNA expression. This mechanism also allows the uncoupling of expression of individual miRNAs from within a single pri-miRNA cluster. In exploring the mechanism of the dynamic posttranscriptional control of pri-miR-17˜92 miRNA expression during ESC differentiation, a new miRNA biogenesis intermediate that has been termed progenitor miRNA (pro-miRNA) was discovered. Specific cleavage of an autoinhibitory 5′ RNA fragment is required to selectively license Microprocessor-mediated production of most pre-miRNAs from pri-miR-17˜92. A biochemical approach was employed to identify possible factors involved in pro-miRNA biogenesis and showed using a variety of approaches that the CPSF3 ribonuclease as well as the spicing factor ISY1 (and other U2 snRNP components) are required for pro-miRNA biogenesis and selective expression of all miRNAs within the cluster except for miR-92. Developmentally regulated ISY1 expression was found to be critical for controlling expression of miRNAs from pri-miR-17˜92 during ESC differentiation.

The identification of pro-miRNA as a novel biogenesis intermediate upstream of Microprocessor challenges the current two-step processing model for miRNA biogenesis. This adds an additional regulatory step for the posttranscriptional control of miR-17˜92 expression. It will therefore be interesting to explore the more widespread relevance of pro-miRNA intermediates in the miRNA biogenesis pathway. In this regard, large, partially processed, pri-miRNAs have been observed in mouse ESCs and it is tempting to speculate that these might also represent pro-miRNA intermediates in the miRNA biogenesis pathway (Houbaviy et al., 2005). Ongoing and future research effects will uncover the widespread relevance of this pathway. Considering that pro-miRNA genesis is the key regulatory step controlling miR-17˜92 expression, it is likely that this paradigm will apply to other miRNAs and in different cellular contexts. This also highlights the complexity of posttranscriptional control of miRNA expression that involves the coupling and coordinated action of multiple cellular machineries that might assemble as part of an integrated ‘holo-factory’ on pri-miRNAs for precise and developmental control of miRNA expression (FIG. 7H) (Pawlicki and Steitz, 2010). The results also highlight a potential limitation of in vitro Microprocessor assays that typically utilize artificially truncated ‘pri-miRNAs’ substrates and therefore might miss important regulatory mechanisms that exists in cells (Han et al., 2006).

Several factors have been identified that either promote or suppress Microprocessor activity for different subsets of miRNAs (Siomi and Siomi, 2010). However autoregulation of miRNA biogenesis mediated by sequences present in the pri-miRNA has not so far been described. The present data implicate RNA conformation in the selective inhibition of Microprocessor cleavage of pri-miR-17˜92. In the presence of the autoinhibitory 5′ fragment the pri-miR-17˜92 adopts a restrictive conformation that blocks processing of all pre-miRNAs in the cluster except for pre-miR-92. Cleavage of the pri-miR-17˜92 to remove the 5′ inhibitory region likely permits the adoption of a less highly structured pri-miR-17˜92 conformation that favors cleavage by Microprocessor. Indeed, a role for RNA tertiary structure in regulating miR-17˜92 has been previously suggested (Chakraborty et al., 2012; Chaulk et al., 2011; Chaulk et al., 2014). However, those reports deal exclusively with the miR-17˜92 cluster without any flanking sequences (i.e., the equivalent of the pro-miRNA). Also, the proposed model whereby the miR-17˜92 cluster adopts a globular tertiary structure with pre-miR-19b and pre-miR-92 at the core does not correlate well with the relative abundance of mature miRNAs in cells since miR-19b, and/or miR-92 are often the most highly expressed members of the cluster. The physiological relevance of this work therefore remains unclear (Chaulk et al., 2011). The identification of two complementary repression domains that nucleate the formation of a repressive higher order RNA conformation to control miRNA biogenesis might also be a relevant mechanism for the control of other RNAs including protein-coding mRNAs.

The exact mechanism and full repertoire of factors responsible for the coupling of pri-miR-17˜92 transcription, recruitment of CPSF3, ISY1, and other spliceosome subunits for the precise cleavage of the autoinhibitory RNA fragment, and subsequent processing by Microprocessor remain active areas of investigation. CPSF3 is known to be required for the cleavage (and subsequent polyadenylation at the 3′-end) of mRNAs and is also involved in the generation of the 3′ end of (non-polyadenylated) histone mRNAs (Dominski et al., 2005; Mandel et al., 2006). In the latter case, CPSF3 cleavage activity is directed by the U7 small nuclear ribonucleoprotein (snRNP) (Dominski et al., 2005). Although CPSF3 protein is sufficient to specifically cleave pri-miR-17˜92 in vitro, this activity was found to be enhanced by ISY1 complex, and ISY1 is required for pro-miRNA biogenesis in cells. The physical association of CPSF3 with both the U1 SnRNP as well as the U2 snRNP has been reported (Kyburz et al., 2006; Wassarman and Steitz, 1993). Analogous to the processing of histone pre-mRNAs, a role for components of the U2 snRNP was found, and in particular, a poorly characterized protein ISY1 that likely helps recruit and direct CPSF3 activity in vivo. This activity requires the endonuclease CPSF3 but not other complex components. Furthermore, pri-miR-17˜92 cleavage does not lead to polyadenylation since the 5′ fragment is detected specifically in the polyA− RNA fraction suggesting that these activities are uncoupled in this context. Moreover, Drosha is known to physically associate with the spliceosome yet the precise functional relevance of this interaction is not completely understood and might be variable depending on the particular pri-miRNAs (Kataoka et al., 2009; Kim and Kim, 2007; Morlando et al., 2008; Pawlicki and Steitz, 2010). The present model implicates multiple protein complexes and different activities that converge to regulate pro-miRNA biogenesis in a developmentally regulated manner (FIG. 7H). This work examined the developmental regulation of miR-17˜92 expression. Considering the strong links of this miRNA cluster with numerous human malignancies it will be of great interest to further explore the relevance of this control mechanism in the context of cancer. Interestingly, common gain-of-function mutations in SF3B1 as well as other splicing factors have been described in myelodysplastic syndromes, leukemia, as well as other solid tumors (Abdel-Wahab and Levine, 2011; Papaemmanuil et al., 2011; Yoshida et al., 2011). It is believed that pharmacological splicing inhibitors that suppress expression of this oncomiR represent a new cancer therapeutic strategy.

### REFERENCES

Abdel-Wahab, O., and Levine, R. (2011). The spliceosome as an indicted conspirator in myeloid malignancies. Cancer Cell 20, 420-423.
Bartel, D. P. (2009). MicroRNAs: target recognition and regulatory functions. Cell 136, 215-233.
Bian, S., Hong, J., Li, Q., Schebelle, L., Pollock, A., Knauss, J. L., Garg, V., and Sun, T. (2013). MicroRNA cluster miR-17-92 regulates neural stem cell expansion and transition to intermediate progenitors in the developing mouse neocortex. Cell Rep 3, 1398-1406.
Chakraborty, S., Mehtab, S., Patwardhan, A., and Krishnan, Y. (2012). Pri-miR-17-92a transcript folds into a tertiary structure and autoregulates its processing. RNA 18, 1014-1028.
Chang, H. M., Triboulet, R., Thornton, J. E., and Gregory, R. I. (2013). A role for the Perlman syndrome exonuclease Dis312 in the Lin28-let-7 pathway. Nature 497, 244-248.
Chaulk, S. G., Thede, G. L., Kent, O. A., Xu, Z., Gesner, E. M., Veldhoen, R. A., Khanna, S. K., Goping, I. S., MacMillan, A. M., Mendell, J. T., et al. (2011). Role of pri-miRNA tertiary structure in miR-17˜92 miRNA biogenesis. RNA Biol 8, 1105-1114.
Chaulk, S. G., Xu, Z., Glover, M. J., and Fahlman, R. P. (2014). MicroRNA miR-92a-1 biogenesis and mRNA targeting is modulated by a tertiary contact within the miR-17˜92 microRNA cluster. Nucleic acids research 42, 5234-5244.
Concepcion, C. P., Bonetti, C., and Ventura, A. (2012). The microRNA-17-92 family of microRNA clusters in development and disease. Cancer J 18, 262-267.
Conkrite, K., Sundby, M., Mukai, S., Thomson, J. M., Mu, D., Hammond, S. M., and MacPherson, D. (2011). miR-17˜92 cooperates with RB pathway mutations to promote retinoblastoma. Genes Dev 25, 1734-1745.
de Pontual, L., Yao, E., Callier, P., Faivre, L., Drouin, V., Cariou, S., Van Haeringen, A., Genevieve, D., Goldenberg, A., Oufadem, M., et al. (2011). Germline deletion of the miR-17 approximately 92 cluster causes skeletal and growth defects in humans. Nat Genet 43, 1026-1030.
Denli, A. M., Tops, B. B., Plasterk, R. H., Ketting, R. F., and Hannon, G. J. (2004). Processing of primary microRNAs by the Microprocessor complex. Nature 432, 231-235.
Di Leva, G., and Croce, C. M. (2010). Roles of small RNAs in tumor formation. Trends Mol Med 16, 257-267.
Dix, I., Russell, C., Yehuda, S. B., Kupiec, M., and Beggs, J. D. (1999). The identification and characterization of a novel splicing protein, Isy1p, of Saccharomyces cerevisiae. RNA 5, 360-368.
Dominski, Z., Yang, X. C., and Marzluff, W. F. (2005). The polyadenylation factor CPSF-73 is involved in histone-pre-mRNA processing. Cell 123, 37-48.
Faehnle, C. R., Walleshauser, J., and Joshua-Tor, L. (2014). Mechanism of Dis312 substrate recognition in the Lin28-let-7 pathway. Nature.
Gregory, R. I., Yan, K. P., Amuthan, G., Chendrimada, T., Doratotaj, B., Cooch, N., and Shiekhattar, R. (2004). The Microprocessor complex mediates the genesis of microRNAs. Nature 432, 235-240.
Guil, S., and Caceres, J. F. (2007). The multifunctional RNA-binding protein hnRNP A1 is required for processing of miR-18a. Nat Struct Mol Biol 14, 591-596.
Ha, M., and Kim, V. N. (2014). Regulation of microRNA biogenesis. Nat Rev Mol Cell Biol 15, 509-524.
Hagan, J. P., Piskounova, E., and Gregory, R. I. (2009). Lin28 recruits the TUTase Zcchc11 to inhibit let-7 maturation in mouse embryonic stem cells. Nat Struct Mol Biol 16, 1021-1025.
Han, J., Lee, Y., Yeom, K. H., Nam, J. W., Heo, I., Rhee, J. K., Sohn, S. Y., Cho, Y., Zhang, B. T., and Kim, V. N. (2006). Molecular basis for the recognition of primary microRNAs by the Drosha-DGCR8 complex. Cell 125, 887-901.
He, L., Thomson, J. M., Hemann, M. T., Hernando-Monge, E., Mu, D., Goodson, S., Powers, S., Cordon-Cardo, C., Lowe, S. W., Hannon, G. J., et al. (2005). A microRNA polycistron as a potential human oncogene. Nature 435, 828-833.
Heo, I., Joo, C., Cho, J., Ha, M., Han, J., and Kim, V. N. (2008). Lin28 mediates the terminal uridylation of let-7 precursor MicroRNA. Mol Cell 32, 276-284.
Heo, I., Joo, C., Kim, Y. K., Ha, M., Yoon, M. J., Cho, J., Yeom, K. H., Han, J., and Kim, V. N. (2009). TUT4 in concert with Lin28 suppresses microRNA biogenesis through pre-microRNA uridylation. Cell 138, 696-708.
Houbaviy, H. B., Dennis, L., Jaenisch, R., and Sharp, P. A. (2005). Characterization of a highly variable eutherian microRNA gene. RNA 11, 1245-1257.
Kataoka, N., Fujita, M., and Ohno, M. (2009). Functional association of the Microprocessor complex with the spliceosome. Mol Cell Biol 29, 3243-3254.
Kim, Y. K., and Kim, V. N. (2007). Processing of intronic microRNAs. EMBO J 26, 775-783.
Kyburz, A., Friedlein, A., Langen, H., and Keller, W. (2006). Direct interactions between subunits of CPSF and the U2 snRNP contribute to the coupling of pre-mRNA 3′ end processing and splicing. Mol Cell 23, 195-205.
Li, Y., Vecchiarelli-Federico, L. M., Li, Y. J., Egan, S. E., Spaner, D., Hough, M. R., and Ben-David, Y. (2012). The miR-17-92 cluster expands multipotent hematopoietic progenitors whereas imbalanced expression of its individual oncogenic miRNAs promotes leukemia in mice. Blood 119, 4486-4498.
Mandel, C. R., Kaneko, S., Zhang, H., Gebauer, D., Vethantham, V., Manley, J. L., and Tong, L. (2006). Polyadenylation factor CPSF-73 is the pre-mRNA 3′-end-processing endonuclease. Nature 444, 953-956.
Marrone, A. K., Stolz, D. B., Bastacky, S. I., Kostka, D., Bodnar, A. J., and Ho, J. (2014). MicroRNA-17˜92 is required for nephrogenesis and renal function. J Am Soc Nephrol 25, 1440-1452.
Mavrakis, K. J., Wolfe, A. L., Oricchio, E., Palomero, T., de Keersmaecker, K., McJunkin, K., Zuber, J., James, T., Khan, A. A., Leslie, C. S., et al. (2010). Genome-wide RNA-mediated interference screen identifies miR-19 targets in Notch-induced T-cell acute lymphoblastic leukaemia. Nat Cell Biol 12, 372-379.
Mendell, J. T. (2008). miRiad roles for the miR-17-92 cluster in development and disease. Cell 133, 217-222.
Mendell, J. T., and Olson, E. N. (2012). MicroRNAs in stress signaling and human disease. Cell 148, 1172-1187.
Mori, M., Triboulet, R., Mohseni, M., Schlegelmilch, K., Shrestha, K., Camargo, F. D., and Gregory, R. I. (2014). Hippo Signaling Regulates Microprocessor and Links Cell-Density-Dependent miRNA Biogenesis to Cancer. Cell 156, 893-906.
Morlando, M., Ballarino, M., Gromak, N., Pagano, F., Bozzoni, I., and Proudfoot, N. J. (2008). Primary microRNA transcripts are processed co-transcriptionally. Nat Struct Mol Biol 15, 902-909.
Mu, P., Han, Y. C., Betel, D., Yao, E., Squatrito, M., Ogrodowski, P., de Stanchina, E., D'Andrea, A., Sander, C., and Ventura, A. (2009). Genetic dissection of the miR-17˜92 cluster of microRNAs in Myc-induced B-cell lymphomas. Genes Dev 23, 2806-2811.
Nam, Y., Chen, C., Gregory, R. I., Chou, J. J., and Sliz, P. (2011). Molecular Basis for Interaction of let-7 MicroRNAs with Lin28. Cell 147, 1080-1091.
Newman, M. A., Thomson, J. M., and Hammond, S. M. (2008). Lin-28 interaction with the Let-7 precursor loop mediates regulated microRNA processing. RNA 14, 1539-1549.
Nittner, D., Lambertz, I., Clermont, F., Mestdagh, P., Kohler, C., Nielsen, S. J., Jochemsen, A., Speleman, F., Vandesompele, J., Dyer, M. A., et al. (2012). Synthetic lethality between Rb, p53 and Dicer or miR-17-92 in retinal progenitors suppresses retinoblastoma formation. Nat Cell Biol 14, 958-965.
O'Donnell, K. A., Wentzel, E. A., Zeller, K. I., Dang, C. V., and Mendell, J. T. (2005). c-Myc-regulated microRNAs modulate E2F1 expression. Nature 435, 839-843.
Olive, V., Bennett, M. J., Walker, J. C., Ma, C., Jiang, I., Cordon-Cardo, C., Li, Q. J., Lowe, S. W., Hannon, G. J., and He, L. (2009). miR-19 is a key oncogenic component of mir-17-92. Genes Dev 23, 2839-2849.
Olive, V., Sabio, E., Bennett, M. J., De Jong, C. S., Biton, A., McGann, J. C., Greaney, S. K., Sodir, N. M., Zhou, A. Y., Balakrishnan, A., et al. (2013). A component of the mir-17-92 polycistronic oncomir promotes oncogene-dependent apoptosis. Elife 2, e00822.
Papaemmanuil, E., Cazzola, M., Boultwood, J., Malcovati, L., Vyas, P., Bowen, D., Pellagatti, A., Wainscoat, J. S., Hellstrom-Lindberg, E., Gambacorti-Passerini, C., et al. (2011). Somatic SF3B1 mutation in myelodysplasia with ring sideroblasts. N Engl J Med 365, 1384-1395.
Patel, V., Williams, D., Hajarnis, S., Hunter, R., Pontoglio, M., Somlo, S., and Igarashi, P. (2013). miR-17˜92 miRNA cluster promotes kidney cyst growth in polycystic kidney disease. Proc Natl Acad Sci USA 110, 10765-10770.
Pawlicki, J. M., and Steitz, J. A. (2010). Nuclear networking fashions pre-messenger RNA and primary microRNA transcripts for function. Trends Cell Biol 20, 52-61.
Piskounova, E., Polytarchou, C., Thornton, J. E., Lapierre, R. J., Pothoulakis, C., Hagan, J. P., Iliopoulos, D., and Gregory, R. I. (2011). Lin28A and Lin28B Inhibit let-7 MicroRNA Biogenesis by Distinct Mechanisms. Cell 147, 1066-1079.
Rybak, A., Fuchs, H., Smirnova, L., Brandt, C., Pohl, E. E., Nitsch, R., and Wulczyn, F. G. (2008). A feedback loop comprising lin-28 and let-7 controls pre-let-7 maturation during neural stem-cell commitment. Nat Cell Biol 10, 987-993.
Sandhu, S. K., Fassan, M., Volinia, S., Lovat, F., Balatti, V., Pekarsky, Y., and Croce, C. M. (2013). B-cell malignancies in microRNA Emu-miR-17˜92 transgenic mice. Proc Natl Acad Sci USA 110, 18208-18213.
Siomi, H., and Siomi, M. C. (2010). Posttranscriptional regulation of microRNA biogenesis in animals. Mol Cell 38, 323-332.
Thornton, J. E., Chang, H. M., Piskounova, E., and Gregory, R. I. (2012). Lin28-mediated control of let-7 microRNA expression by alternative TUTases Zcchc11 (TUT4) and Zcchc6 (TUT7). RNA 18, 1875-1885.
Thornton, J. E., Du, P., Jing, L., Sjekloca, L., Lin, S., Grossi, E., Sliz, P., Zon, L. I., and Gregory, R. I. (2014). Selective microRNA uridylation by Zcchc6 (TUT7) and Zcchc11 (TUT4). Nucleic acids research.
Ustianenko, D., Hrossova, D., Potesil, D., Chalupnikova, K., Hrazdilova, K., Pachernik, J., Cetkovska, K., Uldrijan, S., Zdrahal, Z., and Vanacova, S. (2013). Mammalian DIS3L2 exoribonuclease targets the uridylated precursors of let-7 miRNAs. RNA 19, 1632-1638.
Ventura, A., Young, A. G., Winslow, M. M., Lintault, L., Meissner, A., Erkeland, S. J., Newman, J., Bronson, R. T., Crowley, D., Stone, J. R., et al. (2008). Targeted deletion reveals essential and overlapping functions of the miR-17 through 92 family of miRNA clusters. Cell 132, 875-886.
Villa, T., and Guthrie, C. (2005). The Isy1p component of the NineTeen complex interacts with the ATPase Prp16p to regulate the fidelity of pre-mRNA splicing. Genes Dev 19, 1894-1904.
Viswanathan, S. R., Daley, G. Q., and Gregory, R. I. (2008). Selective blockade of microRNA processing by Lin28. Science 320, 97-100.
Viswanathan, S. R., Powers, J. T., Einhorn, W., Hoshida, Y., Ng, T. L., Toffanin, S., O'Sullivan, M., Lu, J., Phillips, L. A., Lockhart, V. L., et al. (2009). Lin28 promotes transformation and is associated with advanced human malignancies. Nat Genet 41, 843-848.
Wassarman, K. M., and Steitz, J. A. (1993). Association with terminal exons in pre-mRNAs: a new role for the U1 snRNP? Genes Dev 7,647-659.
Yoshida, K., Sanada, M., Shiraishi, Y., Nowak, D., Nagata, Y., Yamamoto, R., Sato, Y., Sato-Otsubo, A., Kon, A., Nagasaki, M., et al. (2011). Frequent pathway mutations of splicing machinery in myelodysplasia. Nature 478, 64-69.

### Example 2

### Introduction

As described below, evidence was found for the widespread posttranscriptional control of pri-miR-17˜92 expression that correlated with elevated ISY1 expression in human primary tumors, and that ISY1 knockdown caused decreased expression of oncomiRs from this cluster in human cancer cells.

### Methods

Human cancer data was downloaded from the TCGA database (https://tcga-data.nci.nih.gov/tcga/tcgaHome2.jsp). R language was used for the statistics analysis. H1299 and A549 cells were cultured in DMEM with 15% (v/v) FBS (Gregory et al., 2004). Lipofectamine 2000 (Invitrogen) was used for both DNA and siRNA transfections according to the manufacturer's instructions.

### Results

Posttranscriptional Control of miR-17˜92 Expression in Human Cancer

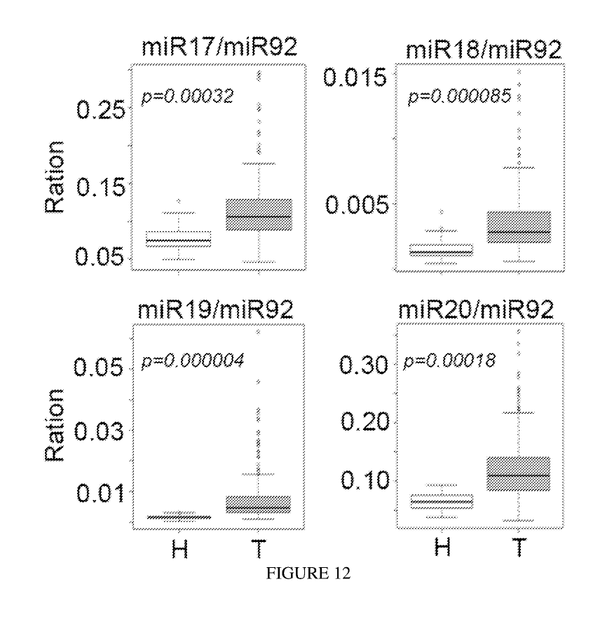

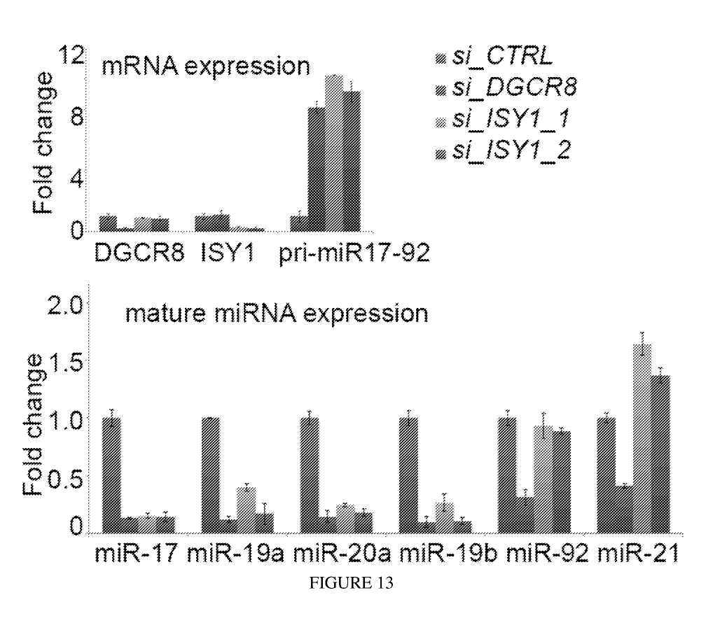

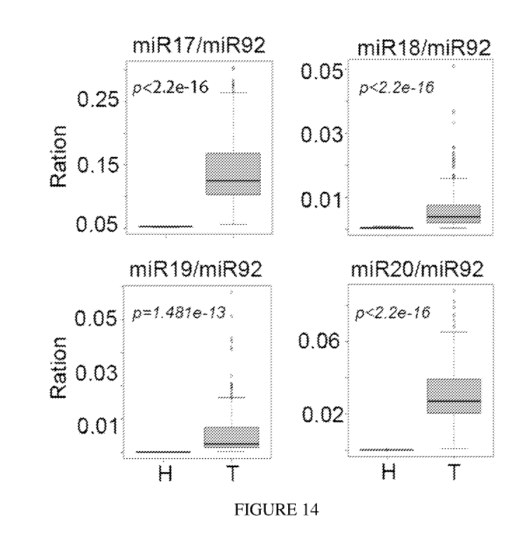

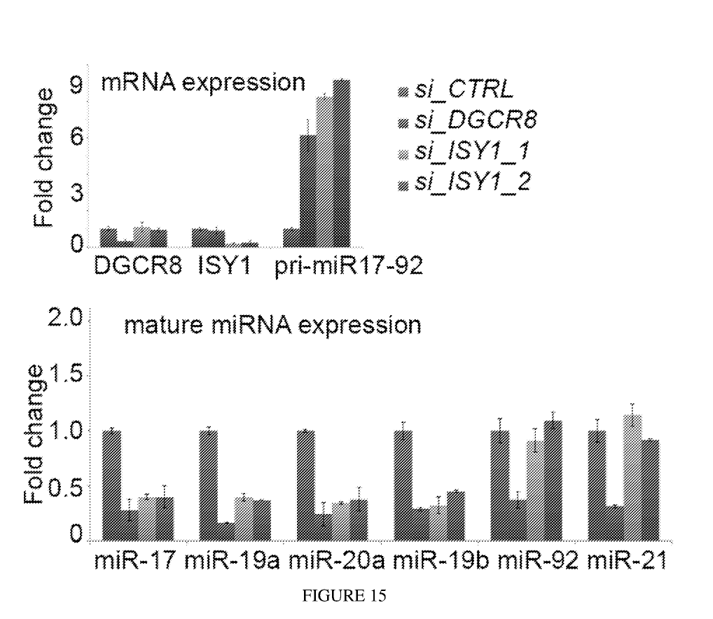

Since miRNAs from the pri-miR-17˜92 promote tumorigenesis are overexpressed in a variety of different cancer types it was next determined whether expression of these miRNAs might be regulated posttranscriptionally in human cancer. Small RNA sequencing data from The Cancer Genome Atlas (TCGA) was analyzed and it was found that the relative expression of miR-17, -18, -19, and -20 is elevated compared to miR-92 in a variety of primary human tumors relative to the corresponding normal tissue. Since these miRNAs are processed from a common pri-miRNA, these data support that posttranscriptional mechanisms might underlie the elevated oncomiR expression in human lung squamous cell carcinoma (FIG. 12) and colon adenocarcinoma (FIG. 14) as well as several other primary tumor types (data not shown). Next the possible role of ISY1 was analyzed in this context. ISY1 knockdown in human lung cancer cell lines was shown to cause selective decreased expression of miR-17, -19a, -19b, and -20 (and not miR-92) supporting that the pathway uncovered in mouse ESCs is evolutionarily conserved and relevant to human disease (FIGS. 13 and 15).

### Discussion

This analysis of primary human tumors and cancer cell lines indicate that this pathway is exploited for increased oncomiR expression in cancer. This suggests that targeting the pathway, e.g., using splicesome inhibitors, such as inhibitors of ISY1, SF3B1, or CPSF3.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of treating cancer, the method comprising:
administering to a subject having cancer an effective amount of an inhibitor of CPSF3, ISY1, or SF3B1.

2. The method of claim 1, wherein the inhibitor is a small molecule, an antisense oligonucleotide, a small interfering RNA (siRNA), a microRNA (miRNA), or an antibody.

3. The method of claim 1, wherein the inhibitor of SF3B1 is selected from the group consisting of FR901463, FR901464, FR901465, spliceostatin A (SSA), a sudemycin, a meayamycin; a pladienolide and GEX1.

4. The method of any one of claims 1 to 3, wherein the cancer is a cancer associated with upregulation of oncomiR1.

5. The method of claim 4, wherein the upregulation of oncomiR1 includes upregulation of one or more of miR-17, miR-18a, miR-19a, miR-20a, or miR-19b.

6. A method of screening for an inhibitor of microRNA (miRNA) biogenesis, the method comprising:
contacting a cell expressing a primary microRNA 17˜92 (pri-miR-17˜92) with a candidate substance;
measuring a ratio of the level of miR-17, miR-18a, miR-19a, miR-20a, and/or miR-19b to the level of miR-92; and
identifying the candidate substance as an inhibitor of miRNA biogenesis if the ratio is decreased compared to a control ratio.

7. The method of claim 6, wherein the measuring comprises a luciferase assay.

8. The method of claim 7, wherein the luciferase assay comprises use of a Renilla Luciferase gene, wherein a 3′UTR of the Renilla Luciferase gene contains a pri-miR-17˜92, or a fragment thereof.

9. The method of any one of claims 6 to 8, wherein the control ratio is the ratio in a cell that has not been contacted with the candidate substance.

10. The method of any one of claims 6 to 9, wherein the candidate substance is a small molecule.

11. A variant primary microRNA (pri-miRNA) that is incapable of forming a progenitor-microRNA (pro-miRNA).

12. The variant pri-miRNA of claim 11, wherein the variant pri-miRNA is not processed by CPSF3.

13. The variant pri-miRNA of claim 11 or 12, comprising a mutation in a CPSF3 cleavage domain.

14. The variant pri-miRNA of claim 11 or 12, comprising a mutation in the sequence CAGUCAGAAUAAUGU.

15. The variant pri-miRNA of claim of claim 12, wherein the mutation is a mutation in the second A and/or the second C in the sequence CAGUCAGAAUAAUGU.

16. The variant pri-miRNA of any one of claims 11 to 15, wherein the variant pri-miRNA is a variant pri-miR-17˜92.

17. A vector comprising a coding sequence encoding the variant pri-miRNA of any one of claims 11 to 16.

18. A method of treating cancer in a subject, the method comprising:
administering to the subject an effective amount of an agent that inhibits formation of a progenitor-microRNA (pro-miRNA).

19. The method of claim 18, wherein the agent is an inhibitor of CPSF3, ISY1, or SF3B1.

20. A method of reducing progenitor-microRNA (pro-miRNA) levels in a cell, the method comprising:
contacting the cell with an agent that inhibits formation of a progenitor-microRNA (pro-miRNA).

21. The method of claim 20, wherein contacting the cell with the agent reduces the levels of one or more of miR-17, miR-18a, miR-19a, miR-20a, or miR-19b in the cell.

22. The method of claim 20 or 21, wherein the agent is an inhibitor of CPSF3, ISY1, or SF3B1.

23. The method of any one of claims 20 to 22, wherein the cell is a cancer cell.
